SEQUENCE LISTING (191 sequences)

SEQ ID NO: 1 - protein, 8 aa, Artificial
  MISC_FEATURE: synthetic peptide 1 (Alpha-1-antitrypsin 1-1 [Precursor])
LAQIHFPR

SEQ ID NO: 2 - protein, 10 aa, Artificial
  MISC_FEATURE: synthetic peptide 2 (Alpha-1-antitrypsin 1-1 [Precursor])
TLMSPLGITR

SEQ ID NO: 3 - protein, 9 aa, Artificial
  MISC_FEATURE: synthetic peptide 3 (Alpha-1-antitrypsin 1-1 [Precursor])
RLAQIHFPR

SEQ ID NO: 4 - protein, 10 aa, Artificial
  MISC_FEATURE: synthetic peptide 4 (Alpha-1-antitrypsin 1-1 [Precursor])
MQHLEQTLSK

SEQ ID NO: 5 - protein, 10 aa, Artificial
  MISC_FEATURE: synthetic peptide 5 (Alpha-1-antitrypsin 1-1 [Precursor])
ELISKFLLNR

SEQ ID NO: 6 - protein, 19 aa, Artificial
  MISC_FEATURE: synthetic peptide 6 (Alpha-1-antitrypsin 1-1 [Precursor])
IFNNGADLSGITEENAPLK

SEQ ID NO: 7 - protein, 19 aa, Artificial
  MISC_FEATURE: synthetic peptide 7 (Alpha-1-antitrypsin 1-1 [Precursor])
NHYQAEVFSVNFAESEEAK

SEQ ID NO: 8 - protein, 22 aa, Artificial
  MISC_FEATURE: synthetic peptide 8 (Alpha-1-antitrypsin 1-1 [Precursor])
DQSPASHEIATNLGDFAISLYR

SEQ ID NO: 9 - protein, 22 aa, Artificial
  MISC_FEATURE: synthetic peptide 9 (Alpha-1-antitrypsin 1-1 [Precursor])
KPFDPENTEEAEFHVDESTTVK

SEQ ID NO: 10 - protein, 22 aa, Artificial
  MISC_FEATURE: synthetic peptide 10 (Alpha-1-antitrypsin 1-1 [Precursor])
FDHPFLFIIFEEHTQSPLFVGK

SEQ ID NO: 11 - protein, 16 aa, Artificial
  MISC_FEATURE: synthetic peptide 11 (Alpha-2-macroglobulin [Precursor])
APFALQVNTLPLNFDK

SEQ ID NO: 12 - protein, 8 aa, Artificial
  MISC_FEATURE: synthetic peptide 12 (Properdin)
TCDHPAPR

SEQ ID NO: 13 - protein, 10 aa, Artificial
  MISC_FEATURE: synthetic peptide 13 (Properdin)
QRLCTPLLPK

SEQ ID NO: 14 - protein, 12 aa, Artificial
  MISC_FEATURE: synthetic peptide 14 (Properdin)
HGGPFCAGDATR

SEQ ID NO: 15 - protein, 14 aa, Artificial
  MISC_FEATURE: synthetic peptide 15 (Properdin)
MSINCEGTPGQQSR

SEQ ID NO: 16 - protein, 16 aa, Artificial
  MISC_FEATURE: synthetic peptide 16 (Properdin)
LRMSINCEGTPGQQSR

SEQ ID NO: 17 - protein, 18 aa, Artificial
  MISC_FEATURE: synthetic peptide 17 (Properdin)
HGGPFCAGDATRNQMCNK

SEQ ID NO: 18 - protein, 19 aa, Artificial
  MISC_FEATURE: synthetic peptide 18 (Properdin)
CGGHCPGEAQQSQACDTQK

SEQ ID NO: 19 - protein, 24 aa, Artificial
  MISC_FEATURE: synthetic peptide 19 (Properdin)
SCSAPAPSHQPPGKPCSGPAYEHK

SEQ ID NO: 20 - protein, 7 aa, Artificial
  MISC_FEATURE: synthetic peptide 20 (Transthyretin [Precursor])
FVEGVYR

SEQ ID NO: 21 - protein, 13 aa, Artificial
  MISC_FEATURE: synthetic peptide 21 (Transthyretin [Precursor])
TSEGSWEPFASGK

SEQ ID NO: 22 - protein, 15 aa, Artificial
  MISC_FEATURE: synthetic peptide 22 (Transthyretin [Precursor])
TAESGELHGLTTDEK

SEQ ID NO: 23 - protein, 23 aa, Artificial
  MISC_FEATURE: synthetic peptide 23 (Transthyretin [Precursor])
TLGISPFHEFADVVFTANDSGHR

SEQ ID NO: 24 - protein, 24 aa, Artificial
  MISC_FEATURE: synthetic peptide 24 (Transthyretin [Precursor])
HYTIAALLSPYSYSTTAVVSNPQN

SEQ ID NO: 25 - protein, 15 aa, Artificial
  MISC_FEATURE: synthetic peptide 25 (Alpha-1-acid glycoprotein 8 [Precursor])
YEGGVETFAHLIVLR

SEQ ID NO: 26 - protein, 7 aa, Artificial
  MISC_FEATURE: synthetic peptide 26 (Apolipoprotein A-I [Precursor])
LQELQGR

SEQ ID NO: 27 - protein, 7 aa, Artificial
  MISC_FEATURE: synthetic peptide 27 (Apolipoprotein A-I [Precursor])
EDVELYR

SEQ ID NO: 28 - protein, 9 aa, Artificial
  MISC_FEATURE: synthetic peptide 28 (Apolipoprotein A-I [Precursor])
ARPALEDLR

SEQ ID NO: 29 - protein, 9 aa, Artificial
  MISC_FEATURE: synthetic peptide 29 (Apolipoprotein A-I [Precursor])
LSPVAEEFR

SEQ ID NO: 30 - protein, 9 aa, Artificial
  MISC_FEATURE: synthetic peptide 30 (Apolipoprotein A-I [Precursor])
QKLQELQGR

SEQ ID NO: 31 - protein, 9 aa, Artificial
  MISC_FEATURE: synthetic peptide 31(Apolipoprotein A-I [Precursor])
WKEDVELYR

SEQ ID NO: 32 - protein, 10 aa, Artificial
  MISC_FEATURE: synthetic peptide 32 (Apolipoprotein A-I [Precursor])
VQPYLDEFQK

SEQ ID NO: 33 - protein, 11 aa, Artificial
  MISC_FEATURE: synthetic peptide 33 (Apolipoprotein A-I [Precursor])
TQLAPHSEQMR

SEQ ID NO: 34 - protein, 11 aa, Artificial
  MISC_FEATURE: synthetic peptide 34 (Apolipoprotein A-I [Precursor])
LSPVAEEFRDR

SEQ ID NO: 35 - protein, 11 aa, Artificial
  MISC_FEATURE: synthetic peptide 35 (Apolipoprotein A-I [Precursor])
SNPTLNEYHTR

SEQ ID NO: 36 - protein, 13 aa, Artificial
  MISC_FEATURE: synthetic peptide 36 (Apolipoprotein A-I [Precursor])
VAPLGAELQESAR

SEQ ID NO: 37 - protein, 11 aa, Artificial
  MISC_FEATURE: synthetic peptide 37 (Apolipoprotein A-I [Precursor])
QEMNKDLEEVK

SEQ ID NO: 38 - protein, 13 aa, Artificial
  MISC_FEATURE: synthetic peptide 38 (Apolipoprotein A-I [Precursor])
VKDFANVYVDAVK

SEQ ID NO: 39 - protein, 16 aa, Artificial
  MISC_FEATURE: synthetic peptide 39 (Apolipoprotein A-I [Precursor])
LQELQGRLSPVAEEFR

SEQ ID NO: 40 - protein, 19 aa, Artificial
  MISC_FEATURE: synthetic peptide 40 (Apolipoprotein C-III [Precursor])
TVQDALSSVQESDIAVVAR

SEQ ID NO: 41 - protein, 6 aa, Artificial
  MISC_FEATURE: synthetic peptide 41 (Beta-2-glycoprotein 1 [Precursor])
IHFYCK

SEQ ID NO: 42 - protein, 9 aa, Artificial
  MISC_FEATURE: synthetic peptide 42 (Beta-2-glycoprotein 1 [Precursor])
ATVLYQGMR

SEQ ID NO: 43 - protein, 10 aa, Artificial
  MISC_FEATURE: synthetic peptide 43 (Beta-2-glycoprotein 1 [Precursor])
ITCPPPPVPK

SEQ ID NO: 44 - protein, 10 aa, Artificial
  MISC_FEATURE: synthetic peptide 44 (Beta-2-glycoprotein 1 [Precursor])
WSPDIPACAR

SEQ ID NO: 45 - protein, 11 aa, Artificial
  MISC_FEATURE: synthetic peptide 45 (Beta-2-glycoprotein 1 [Precursor])
DGTIEIPSCFK

SEQ ID NO: 46 - protein, 10 aa, Artificial
  MISC_FEATURE: synthetic peptide 46 (Beta-2-glycoprotein 1 [Precursor])
CSYTVEAHCR

SEQ ID NO: 47 - protein, 11 aa, Artificial
  MISC_FEATURE: synthetic peptide 47 (Beta-2-glycoprotein 1 [Precursor])
TGTWSFLPTCR

SEQ ID NO: 48 - protein, 14 aa, Artificial
  MISC_FEATURE: synthetic peptide 48 (Beta-2-glycoprotein 1 [Precursor])
VCPFAGILENGIVR

SEQ ID NO: 49 - protein, 14 aa, Artificial
  MISC_FEATURE: synthetic peptide 49 (Beta-2-glycoprotein 1 [Precursor])
IQEQFKNGMMHGDK

SEQ ID NO: 50 - protein, 15 aa, Artificial
  MISC_FEATURE: synthetic peptide 50 (Beta-2-glycoprotein 1 [Precursor])
FTCPLTGMWPINTLR

SEQ ID NO: 51 - protein, 17 aa, Artificial
  MISC_FEATURE: synthetic peptide 51 (Beta-2-glycoprotein 1 [Precursor])
ICPKPDDLPFATVVPLK

SEQ ID NO: 52 - protein, 20 aa, Artificial
  MISC_FEATURE: synthetic peptide 52 (Beta-2-glycoprotein 1 [Precursor])
TSYDPGEQIVYSCKPGYVSR

SEQ ID NO: 53 - protein, 23 aa, Artificial
  MISC_FEATURE: synthetic peptide 53 (Beta-2-glycoprotein 1 [Precursor])
CPFPPRPENGYVNYPAKPVLLYK

SEQ ID NO: 54 - protein, 17 aa, Artificial
  MISC_FEATURE: synthetic peptide 54 (Glutathione peroxidase 3 [Precursor])
YVRPGGGFVPNFQLFEK

SEQ ID NO: 55 - protein, 10 aa, Artificial
  MISC_FEATURE: synthetic peptide 55 (Plasma retinol-binding protein [Precursor])
DPNGLSPETR

SEQ ID NO: 56 - protein, 10 aa, Artificial
  MISC_FEATURE: synthetic peptide 56 (Plasma retinol-binding protein [Precursor])
YWGVASFLQR

SEQ ID NO: 57 - protein, 10 aa, Artificial
  MISC_FEATURE: synthetic peptide 57 (Plasma retinol-binding protein [Precursor])
QRQEELCLER

SEQ ID NO: 58 - protein, 18 aa, Artificial
  MISC_FEATURE: synthetic peptide 58 (Plasma retinol-binding protein [Precursor])
LQNLDGTCADSYSFVFSR

SEQ ID NO: 59 - protein, 21 aa, Artificial
  MISC_FEATURE: synthetic peptide 59 (Plasma retinol-binding protein [Precursor])
KDPEGLFLQDNIIAEFSVDEK

SEQ ID NO: 60 - protein, 20 aa, Artificial
  MISC_FEATURE: synthetic peptide 60 (Serum amyloid P-component [Precursor])
APPSIVLGQEQDNYGGGFQR

SEQ ID NO: 61 - protein, 8 aa, Artificial
  MISC_FEATURE: synthetic peptide 61 (Apolipoprotein E [Precursor])
EVQAAQAR

SEQ ID NO: 62 - protein, 6 aa, Artificial
  MISC_FEATURE: synthetic peptide 62 (Apolipoprotein E [Precursor])
FWDYLR

SEQ ID NO: 63 - protein, 7 aa, Artificial
  MISC_FEATURE: synthetic peptide 63 (Apolipoprotein E [Precursor])
DRLEEVR

SEQ ID NO: 64 - protein, 7 aa, Artificial
  MISC_FEATURE: synthetic peptide 64 (Apolipoprotein E [Precursor])
EHMEEVR

SEQ ID NO: 65 - protein, 9 aa, Artificial
  MISC_FEATURE: synthetic peptide 65 (Apolipoprotein E [Precursor])
LGPLVEQGR

SEQ ID NO: 66 - protein, 9 aa, Artificial
  MISC_FEATURE: synthetic peptide 66 (Apolipoprotein E [Precursor])
LEEVGNQAR

SEQ ID NO: 67 - protein, 8 aa, Artificial
  MISC_FEATURE: synthetic peptide 67 (Apolipoprotein E [Precursor])
QWANLMEK

SEQ ID NO: 68 - protein, 9 aa, Artificial
  MISC_FEATURE: synthetic peptide 68 (Apolipoprotein E [Precursor])
DRAQAFGDR

SEQ ID NO: 69 - protein, 9 aa, Artificial
  MISC_FEATURE: synthetic peptide 69 (Apolipoprotein E [Precursor])
LQAEIFQAR

SEQ ID NO: 70 - protein, 9 aa, Artificial
  MISC_FEATURE: synthetic peptide 70 (Apolipoprotein E [Precursor])
MEEQTQQIR

SEQ ID NO: 71 - protein, 11 aa, Artificial
  MISC_FEATURE: synthetic peptide 71 (Apolipoprotein E [Precursor])
GRLEEVGNQAR

SEQ ID NO: 72 - protein, 13 aa, Artificial
  MISC_FEATURE: synthetic peptide 72 (Apolipoprotein E [Precursor])
TANLGAGAAQPLR

SEQ ID NO: 73 - protein, 11 aa, Artificial
  MISC_FEATURE: synthetic peptide 73 (Apolipoprotein E [Precursor])
SKMEEQTQQIR

SEQ ID NO: 74 - protein, 12 aa, Artificial
  MISC_FEATURE: synthetic peptide 74 (Apolipoprotein E [Precursor])
GWFEPIVEDMHR

SEQ ID NO: 75 - protein, 14 aa, Artificial
  MISC_FEATURE: synthetic peptide 75 (Apolipoprotein E [Precursor])
ELEEQLGPVAEETR

SEQ ID NO: 76 - protein, 15 aa, Artificial
  MISC_FEATURE: synthetic peptide 76 (Apolipoprotein E [Precursor])
NEVHTMLGQSTEEIR

SEQ ID NO: 77 - protein, 15 aa, Artificial
  MISC_FEATURE: synthetic peptide 77 (Alpha-1-acid glycoprotein 8 [Precursor])
YEGGVETFAHLIVLR

SEQ ID NO: 78 - protein, 7 aa, Artificial
  MISC_FEATURE: synthetic peptide 78 (Apolipoprotein A-I [Precursor])
LQELQGR

SEQ ID NO: 79 - protein, 7 aa, Artificial
  MISC_FEATURE: synthetic peptide 79 (Apolipoprotein A-I [Precursor])
EDVELYR

SEQ ID NO: 80 - protein, 9 aa, Artificial
  MISC_FEATURE: synthetic peptide 80 (Apolipoprotein A-I [Precursor])
ARPALEDLR

SEQ ID NO: 81 - protein, 9 aa, Artificial
  MISC_FEATURE: synthetic peptide 81(Apolipoprotein A-I [Precursor])
LSPVAEEFR

SEQ ID NO: 82 - protein, 9 aa, Artificial
  MISC_FEATURE: synthetic peptide 82  (Apolipoprotein A-I [Precursor])
QKLQELQGR

SEQ ID NO: 83 - protein, 9 aa, Artificial
  MISC_FEATURE: synthetic peptide 83 (Apolipoprotein A-I [Precursor])
WKEDVELYR

SEQ ID NO: 84 - protein, 10 aa, Artificial
  MISC_FEATURE: synthetic peptide 84 (Apolipoprotein A-I [Precursor])
VQPYLDEFQK

SEQ ID NO: 85 - protein, 11 aa, Artificial
  MISC_FEATURE: synthetic peptide 85 (Apolipoprotein A-I [Precursor])
TQLAPHSEQMR

SEQ ID NO: 86 - protein, 11 aa, Artificial
  MISC_FEATURE: synthetic peptide 86 (Apolipoprotein A-I [Precursor])
LSPVAEEFRDR

SEQ ID NO: 87 - protein, 11 aa, Artificial
  MISC_FEATURE: synthetic peptide 87 (Apolipoprotein A-I [Precursor])
SNPTLNEYHTR

SEQ ID NO: 88 - protein, 13 aa, Artificial
  MISC_FEATURE: synthetic peptide 88 (Apolipoprotein A-I [Precursor])
VAPLGAELQESAR

SEQ ID NO: 89 - protein, 11 aa, Artificial
  MISC_FEATURE: synthetic peptide 89 (Apolipoprotein A-I [Precursor])
QEMNKDLEEVK

SEQ ID NO: 90 - protein, 13 aa, Artificial
  MISC_FEATURE: synthetic peptide 90 (Apolipoprotein A-I [Precursor])
VKDFANVYVDAVK

SEQ ID NO: 91 - protein, 16 aa, Artificial
  MISC_FEATURE: synthetic peptide 91 (Apolipoprotein A-I [Precursor])
LQELQGRLSPVAEEFR

SEQ ID NO: 92 - protein, 19 aa, Artificial
  MISC_FEATURE: synthetic peptide 92 (Apolipoprotein C-III [Precursor])
TVQDALSSVQESDIAVVAR

SEQ ID NO: 93 - protein, 6 aa, Artificial
  MISC_FEATURE: synthetic peptide 93 (Beta-2-glycoprotein 1 [Precursor])
IHFYCK

SEQ ID NO: 94 - protein, 9 aa, Artificial
  MISC_FEATURE: synthetic peptide 94 (Beta-2-glycoprotein 1 [Precursor])
ATVLYQGMR

SEQ ID NO: 95 - protein, 10 aa, Artificial
  MISC_FEATURE: synthetic peptide 95 (Beta-2-glycoprotein 1 [Precursor])
ITCPPPPVPK

SEQ ID NO: 96 - protein, 10 aa, Artificial
  MISC_FEATURE: synthetic peptide 96 (Beta-2-glycoprotein 1 [Precursor])
WSPDIPACAR

SEQ ID NO: 97 - protein, 11 aa, Artificial
  MISC_FEATURE: synthetic peptide 97 (Beta-2-glycoprotein 1 [Precursor])
DGTIEIPSCFK

SEQ ID NO: 98 - protein, 10 aa, Artificial
  MISC_FEATURE: synthetic peptide 98 (Beta-2-glycoprotein 1 [Precursor])
CSYTVEAHCR

SEQ ID NO: 99 - protein, 11 aa, Artificial
  MISC_FEATURE: synthetic peptide 99 (Beta-2-glycoprotein 1 [Precursor])
TGTWSFLPTCR

SEQ ID NO: 100 - protein, 14 aa, Artificial
  MISC_FEATURE: synthetic peptide 100 (Beta-2-glycoprotein 1 [Precursor])
VCPFAGILENGIVR

SEQ ID NO: 101 - protein, 14 aa, Artificial
  MISC_FEATURE: synthetic peptide 101 (Beta-2-glycoprotein 1 [Precursor])
IQEQFKNGMMHGDK

SEQ ID NO: 102 - protein, 15 aa, Artificial
  MISC_FEATURE: synthetic peptide 102 (Beta-2-glycoprotein 1 [Precursor])
FTCPLTGMWPINTLR

SEQ ID NO: 103 - protein, 17 aa, Artificial
  MISC_FEATURE: synthetic peptide 103 (Beta-2-glycoprotein 1 [Precursor])
ICPKPDDLPFATVVPLK

SEQ ID NO: 104 - protein, 20 aa, Artificial
  MISC_FEATURE: synthetic peptide 104 (Beta-2-glycoprotein 1 [Precursor])
TSYDPGEQIVYSCKPGYVSR

SEQ ID NO: 105 - protein, 23 aa, Artificial
  MISC_FEATURE: synthetic peptide 105 (Beta-2-glycoprotein 1 [Precursor])
CPFPPRPENGYVNYPAKPVLLYK

SEQ ID NO: 106 - protein, 17 aa, Artificial
  MISC_FEATURE: synthetic peptide 106 (Glutathione peroxidase 3 [Precursor])
YVRPGGGFVPNFQLFEK

SEQ ID NO: 107 - protein, 8 aa, Artificial
  MISC_FEATURE: synthetic peptide 107 (Major urinary protein 2 [Precursor])
IEDNGNFR

SEQ ID NO: 108 - protein, 10 aa, Artificial
  MISC_FEATURE: synthetic peptide 108  (Major urinary protein 2 [Precursor]
EKIEDNGNFR

SEQ ID NO: 109 - protein, 11 aa, Artificial
  MISC_FEATURE: synthetic peptide 109  (Major urinary protein 2 [Precursor])
ENIIDLSNANR

SEQ ID NO: 110 - protein, 12 aa, Artificial
  MISC_FEATURE: synthetic peptide 110   (Major urinary protein [Fragment])
FAQLCEEHGILR

SEQ ID NO: 111 - protein, 13 aa, Artificial
  MISC_FEATURE: synthetic peptide 111  (Major urinary protein 2 [Precursor])
DGETFQLMGLYGR

SEQ ID NO: 112 - protein, 15 aa, Artificial
  MISC_FEATURE: synthetic peptide 112  (Major urinary protein 2 [Precursor])
INGEWHTIILASDKR

SEQ ID NO: 113 - protein, 15 aa, Artificial
  MISC_FEATURE: synthetic peptide 113  (Major urinary protein 2 [Precursor])
TDYDNFLMAHLINEK

SEQ ID NO: 114 - protein, 16 aa, Artificial
  MISC_FEATURE: synthetic peptide 114  (Major urinary protein 2 [Precursor])
LFLEQIHVLENSLVLK

SEQ ID NO: 115 - protein, 18 aa, Artificial
  MISC_FEATURE: synthetic peptide 115  (Major urinary protein 2 [Precursor])
AGEYSVTYDGFNTFTIPK

SEQ ID NO: 116 - protein, 8 aa, Artificial
  MISC_FEATURE: synthetic peptide 116 (Apolipoprotein A-IV [Precursor])
GNTEGLQK

SEQ ID NO: 117 - protein, 9 aa, Artificial
  MISC_FEATURE: synthetic peptide 117 (Apolipoprotein A-IV [Precursor])
LQLTPYIQR

SEQ ID NO: 118 - protein, 10 aa, Artificial
  MISC_FEATURE: synthetic peptide 118 (Apolipoprotein A-IV [Precursor])
ALVQQLEQFR

SEQ ID NO: 119 - protein, 10 aa, Artificial
  MISC_FEATURE: synthetic peptide 119 (Apolipoprotein A-IV [Precursor])
QLEQQVEEFR

SEQ ID NO: 120 - protein, 12 aa, Artificial
  MISC_FEATURE: synthetic peptide 120 (Apolipoprotein A-IV [Precursor])
ATIDQNLEDLRR

SEQ ID NO: 121 - protein, 11 aa, Artificial
  MISC_FEATURE: synthetic peptide 121 (Apolipoprotein A-IV [Precursor])
QLEQQVEEFRR

SEQ ID NO: 122 - protein, 13 aa, Artificial
  MISC_FEATURE: synthetic peptide 122 (Apolipoprotein A-IV [Precursor])
LNHQMEGLAFQMK

SEQ ID NO: 123 - protein, 14 aa, Artificial
  MISC_FEATURE: synthetic peptide 123 (Apolipoprotein A-IV [Precursor])
TDVTQQLSTLFQDK

SEQ ID NO: 124 - protein, 18 aa, Artificial
  MISC_FEATURE: synthetic peptide 124 (Apolipoprotein A-IV [Precursor])
LVPFVVQLSGHLAKETER

SEQ ID NO: 125 - protein, 19 aa, Artificial
  MISC_FEATURE: synthetic peptide 125 (Apolipoprotein A-IV [Precursor])
QQLGPNSGEVESHLSFLEK

SEQ ID NO: 126 - protein, 24 aa, Artificial
  MISC_FEATURE: synthetic peptide 126 (Apolipoprotein A-IV [Precursor])
LQEHLKPYAVDLQDQINTQTQEMK

SEQ ID NO: 127 - protein, 18 aa, Artificial
  MISC_FEATURE: synthetic peptide 127 (Apolipoprotein A-IV [Precursor])
LVPFVVQLSGHLAKETER

SEQ ID NO: 128 - protein, 7 aa, Artificial
  MISC_FEATURE: synthetic peptide 128 (Apolipoprotein M)
AFLVTPR

SEQ ID NO: 129 - protein, 6 aa, Artificial
  MISC_FEATURE: synthetic peptide 129 (Apolipoprotein M)
FLLYNR

SEQ ID NO: 130 - protein, 8 aa, Artificial
  MISC_FEATURE: synthetic peptide 130 (Apolipoprotein M)
ETGQGYQR

SEQ ID NO: 131 - protein, 11 aa, Artificial
  MISC_FEATURE: synthetic peptide 131 (A-Raf proto-oncogene)
SPHSKLPSEQR

SEQ ID NO: 132 - protein, 9 aa, Artificial
  MISC_FEATURE: synthetic peptide 132 (Fetuin-B [Precursor])
LVVLPFPGK

SEQ ID NO: 133 - protein, 9 aa, Artificial
  MISC_FEATURE: synthetic peptide 133 (Fetuin-B [Precursor])
DGYMLSLNR

SEQ ID NO: 134 - protein, 9 aa, Artificial
  MISC_FEATURE: synthetic peptide 133 (Fetuin-B [Precursor])
  MISC_FEATURE: synthetic peptide 134 (Fetuin-B [Precursor])
AMFHINKPR

SEQ ID NO: 135 - protein, 18 aa, Artificial
  MISC_FEATURE: synthetic peptide 135 (Fetuin-B [Precursor])
AMNQWVSGPAYYVEYLIK

SEQ ID NO: 136 - protein, 28 aa, Artificial
  MISC_FEATURE: synthetic peptide 136 (Fetuin-B [Precursor])
SQASCSLQHSDSEPVGICQGSTVQSSLR

SEQ ID NO: 137 - protein, 27 aa, Artificial
  MISC_FEATURE: synthetic peptide 137 (Fetuin-B [Precursor])
EHYQEDMGSLFYLTLDVLETDCHVLSR

SEQ ID NO: 138 - protein, 7 aa, Artificial
  MISC_FEATURE: synthetic peptide 138 (Gelsolin [Precursor])
LFACSNR

SEQ ID NO: 139 - protein, 8 aa, Artificial
  MISC_FEATURE: synthetic peptide 139 (Gelsolin [Precursor])
RTPITVVR

SEQ ID NO: 140 - protein, 8 aa, Artificial
  MISC_FEATURE: synthetic peptide 140 (Gelsolin [Precursor])
EPGLQIWR

SEQ ID NO: 141 - protein, 11 aa, Artificial
  MISC_FEATURE: synthetic peptide 141 (Gelsolin [Precursor])
DGGQTAPASIR

SEQ ID NO: 142 - protein, 11 aa, Artificial
  MISC_FEATURE: synthetic peptide 142 (Gelsolin [Precursor])
AGKEPGLQIWR

SEQ ID NO: 143 - protein, 11 aa, Artificial
  MISC_FEATURE: synthetic peptide 143 (Gelsolin [Precursor])
HVVPNEVVVQR

SEQ ID NO: 144 - protein, 11 aa, Artificial
  MISC_FEATURE: synthetic peptide 144 (Gelsolin [Precursor])
SEDCFILDHGR

SEQ ID NO: 145 - protein, 15 aa, Artificial
  MISC_FEATURE: synthetic peptide 145 (Gelsolin [Precursor])
EVQGFESSTFSGYFK

SEQ ID NO: 146 - protein, 17 aa, Artificial
  MISC_FEATURE: synthetic peptide 146 (Gelsolin [Precursor])
QTQVSVLPEGGETPLFK

SEQ ID NO: 147 - protein, 18 aa, Artificial
  MISC_FEATURE: synthetic peptide 147 (Gelsolin [Precursor])
EPAHLMSLFGGKPMIIYK

SEQ ID NO: 148 - protein, 22 aa, Artificial
  MISC_FEATURE: synthetic peptide 148 (Gelsolin [Precursor])
SQHVQVEEGSEPDAFWEALGGK

SEQ ID NO: 149 - protein, 25 aa, Artificial
  MISC_FEATURE: synthetic peptide 149 (Gelsolin [Precursor])
VSNGAGSMSVSLVADENPFAQGALR

SEQ ID NO: 150 - protein, 24 aa, Artificial
  MISC_FEATURE: synthetic peptide 150 (Gelsolin [Precursor])
VPVDPATYGQFYGGDSYIILYNYR

SEQ ID NO: 151 - protein, 25 aa, Artificial
  MISC_FEATURE: synthetic peptide 151 (Gelsolin [Precursor])
NWRDPDQTDGPGLGYLSSHIANVER

SEQ ID NO: 152 - protein, 30 aa, Artificial
  MISC_FEATURE: synthetic peptide 152 (Gelsolin [Precursor])
IEGSNKVPVDPATYGQFYGGDSYIILYNYR

SEQ ID NO: 153 - protein, 11 aa, Artificial
  MISC_FEATURE: synthetic peptide 153 (Gelsolin [Precursor])
HVVPNEVVVQR

SEQ ID NO: 154 - protein, 7 aa, Artificial
  MISC_FEATURE: synthetic peptide 154 (Plaminogen [Precursor])
FVDWIER

SEQ ID NO: 155 - protein, 8 aa, Artificial
  MISC_FEATURE: synthetic peptide 155 (Plaminogen [Precursor])
LILEPNNR

SEQ ID NO: 156 - protein, 8 aa, Artificial
  MISC_FEATURE: synthetic peptide 156 (Plaminogen [Precursor])
SSRPEFYK

SEQ ID NO: 157 - protein, 8 aa, Artificial
  MISC_FEATURE: synthetic peptide 157 (Plaminogen [Precursor])
WSEQTPHR

SEQ ID NO: 158 - protein, 8 aa, Artificial
  MISC_FEATURE: synthetic peptide 158 (Plaminogen [Precursor])
NLEENYCR

SEQ ID NO: 159 - protein, 8 aa, Artificial
  MISC_FEATURE: synthetic peptide 159 (Plaminogen [Precursor])
WEYCDIPR

SEQ ID NO: 160 - protein, 9 aa, Artificial
  MISC_FEATURE: synthetic peptide 160 (Plaminogen [Precursor])
MRDVILFEK

SEQ ID NO: 161 - protein, 8 aa, Artificial
  MISC_FEATURE: synthetic peptide 161 (Plaminogen [Precursor])
WEYCNLKR

SEQ ID NO: 162 - protein, 10 aa, Artificial
  MISC_FEATURE: synthetic peptide 162 (Plaminogen [Precursor])
CEGETDFVCR

SEQ ID NO: 163 - protein, 11 aa, Artificial
  MISC_FEATURE: synthetic peptide 163 (Plaminogen [Precursor])
HSIFTPQTNPR

SEQ ID NO: 164 - protein, 11 aa, Artificial
  MISC_FEATURE: synthetic peptide 164 (Plaminogen [Precursor])
VILGAHEEYIR

SEQ ID NO: 165 - protein, 11 aa, Artificial
  MISC_FEATURE: synthetic peptide 165 (Plaminogen [Precursor])
CQSWAAMFPHR

SEQ ID NO: 166 - protein, 11 aa, Artificial
  MISC_FEATURE: synthetic peptide 166 (Plaminogen [Precursor])
VCNRVEYLNNR

SEQ ID NO: 167 - protein, 12 aa, Artificial
  MISC_FEATURE: synthetic peptide 167 (Plaminogen [Precursor])
GPWCYTTDPSVR

SEQ ID NO: 168 - protein, 14 aa, Artificial
  MISC_FEATURE: synthetic peptide 168 (Plaminogen [Precursor])
GTVSVTVSGKTCQR

SEQ ID NO: 169 - protein, 16 aa, Artificial
  MISC_FEATURE: synthetic peptide 169 (Plaminogen [Precursor])
DIALLKLSRPATITDK

SEQ ID NO: 170 - protein, 16 aa, Artificial
  MISC_FEATURE: synthetic peptide 170 (Plaminogen [Precursor])
VIPACLPSPNYMVADR

SEQ ID NO: 171 - protein, 16 aa, Artificial
  MISC_FEATURE: synthetic peptide 171 (Plaminogen [Precursor])
CTTPPPPPSPTYQCLK

SEQ ID NO: 172 - protein, 16 aa, Artificial
  MISC_FEATURE: synthetic peptide 172 (Plaminogen [Precursor])
TPENFPDAGLEMNYCR

SEQ ID NO: 173 - protein, 17 aa, Artificial
  MISC_FEATURE: synthetic peptide 173 (Plaminogen [Precursor])
NPDGDVNGPWCYTTNPR

SEQ ID NO: 174 - protein, 17 aa, Artificial
  MISC_FEATURE: synthetic peptide 174 (Plaminogen [Precursor])
NPDGEPRPWCFTTDPTK

SEQ ID NO: 175 - protein, 18 aa, Artificial
  MISC_FEATURE: synthetic peptide 175 (Plaminogen [Precursor])
NPDGDKGPWCYTTDPSVR

SEQ ID NO: 176 - protein, 19 aa, Artificial
  MISC_FEATURE: synthetic peptide 176 (Plaminogen [Precursor])
TICYITGWGETQGTFGAGR

SEQ ID NO: 177 - protein, 20 aa, Artificial
  MISC_FEATURE: synthetic peptide 177 (Plaminogen [Precursor])
TAVTAAGTPCQGWAAQEPHR

SEQ ID NO: 178 - protein, 18 aa, Artificial
  MISC_FEATURE: synthetic peptide 178 (Plaminogen [Precursor])
NPDGETAPWCYTTDSQLR

SEQ ID NO: 179 - protein, 19 aa, Artificial
  MISC_FEATURE: synthetic peptide 179 (Plaminogen [Precursor])
VVGGCVANPHSWPWQISLR

SEQ ID NO: 180 - protein, 18 aa, Artificial
  MISC_FEATURE: synthetic peptide 180 (Plaminogen [Precursor])
NPDGEPRPWCFTTDPTKR

SEQ ID NO: 181 - protein, 19 aa, Artificial
  MISC_FEATURE: synthetic peptide 181 (Plaminogen [Precursor])
NPDNDEQGPWCYTTDPDKR

SEQ ID NO: 182 - protein, 24 aa, Artificial
  MISC_FEATURE: synthetic peptide 182
YILQGVTSWGLGCARPNKPGVYVR

SEQ ID NO: 183 - protein, 26 aa, Artificial
  MISC_FEATURE: synthetic peptide 183
FTGQHFCGGTLIAPEWVLTAAHCLEK

SEQ ID NO: 184 - protein, 27 aa, Artificial
  MISC_FEATURE: synthetic peptide 184
WGATFPHVPNYSPSTHPNEGLEENYCR

SEQ ID NO: 185 - protein, 14 aa, Artificial
  MISC_FEATURE: synthetic peptide 185 (Vacuolar protein sorting-associated protein 28)
LEIRAMDEIQPDLR

SEQ ID NO: 186 - protein, 14 aa, Artificial
  MISC_FEATURE: synthetic peptide 186 (Vacuolar protein sorting-associated protein 28)
LEIRAMDEIQPDLR

SEQ ID NO: 187 - protein, 16 aa, Artificial
  MISC_FEATURE: synthetic peptide 187 (Vacuolar protein sorting-associated protein 28)
QVQGSEISSIDEFCRK

SEQ ID NO: 188 - protein, 19 aa, Artificial
  MISC_FEATURE: synthetic peptide 188 (Vacuolar protein sorting-associated protein 28)
YDNMAELFAVVKTMQALEK

SEQ ID NO: 189 - protein, 18 aa, Artificial
  MISC_FEATURE: synthetic peptide 189 (Vacuolar protein sorting-associated protein 28)
AMDEIQPDLRELMETMHR

SEQ ID NO: 190 - protein, 25 aa, Artificial
  MISC_FEATURE: synthetic peptide 190 (Vacuolar protein sorting-associated protein 28)
QTVSQWLQTLSGMSASDELDDSQVR

SEQ ID NO: 191 - protein, 27 aa, Artificial
  MISC_FEATURE: synthetic peptide 191 (Vacuolar protein sorting-associated protein 28)
FHGIPATPGVGAPGNKPELYEEVKLYK

(19) United States
(12) Patent Application Publication
(10) Pub. No.: US 2011/0082089 A1
(43) Pub. Date: Apr. 7, 2011

(54) BIOMARKERS FOR MONITORING OR PREDICTING THE TREATMENT OF CANCER

(76) Inventors: Jürgen Borlak, Lehrte OT Immensen (DE); Bijon Chatterji, Hannover (DE)

(21) Appl. No.: 12/889,882

(22) Filed: Sep. 24, 2010

Related U.S. Application Data
(63) Continuation of application No. PCT/EP2009/002442, filed on Mar. 30, 2009.

(30) Foreign Application Priority Data
Mar. 28, 2008 (EP) 08075242.1

(51) Int. Cl. A61K 38/16; C07K 14/775; C07K 14/00; C07K 16/00; C07K 7/00; A61K 38/04; A61P 35/00; G01N 33/566

(52) U.S. Cl. 514/19.3; 530/359; 530/350; 530/389.1; 530/328; 530/327; 530/326; 530/325; 530/329; 530/324; 436/501

(57) ABSTRACT

We provide for the diagnosis, prognosis and/or treatment monitoring of lung cancer or bronchial dysplasia, and the use thereof for predicting and monitoring therapeutic intervention in dysplasia or cancer patients. According to the invention at least one biomarker selected from the group consisting of APOE, APOC3, A1AT6, A2MG, PROP, TTHY, A1AG8, APOA1, APOH, GPX3, MUP8, RETBP, SAMP, VTDB, S6A11, EGFR, ApoA4, ApoM, a-raf, fetuin B, GSN, PLG, VPS28, and particular peptide sequences derived thereof, is used in the diagnosis, prognosis and/or treatment monitoring of cancer or dysplasia, in particular of lung cancer or the level of at least one of said biomarkers is measured in a body fluid sample, in particular in a blood serum sample, of a patient suffering from or being susceptible to cancer or dysplasia.

Within the context of said biomarkers, the invention concerns a composition for qualifying the c-myc activity in a patient suffering or being susceptible to cancer or for classifying a patient suffering from or being susceptible to lung cancer or bronchial dysplasia, in particular by an in vitro body fluid analysis, and a procedure to screen for and to identify drugs against cancer associated with an increased c-myc activity or against dysplasia or cancer associated with an aberrant EGF receptor tyrosine kinase signalling s provided, wherein in a body fluid sample of a transgenic cancer mouse being treated with a compound to be tested at least one of said biomarkers is determined.

### CROSS-REFERENCE TO RELATED APPLICATIONS

This application is a continuation of PCT International Patent Application No. PCT/EP2009/002442, filed on Mar. 30, 2009, and claiming priority to European Application No. 08075242.1, filed on Mar. 28, 2008. Both applications are incorporated by reference herein.

### INCORPORATION BY REFERENCE OF SEQUENCE LISTING

This application is being filed using the United States Patent and Trademark Office's EFS-Web system, with a sequence listing presented in an ASCII text file. The contents of that text file are incorporated by reference herein. The text file is entitled “100906Sequence.txt” and has a size of 53,932 bytes and a creation date of Sep. 8, 2010.

### BACKGROUND OF THE INVENTION

1. Field of the Invention

Embodiments of the invention are directed to biomarkers for determining the c-myc activity in a subject, and the use thereof for predicting and monitoring therapeutic intervention in cancer patients. Areas of application are the life sciences: biology, biochemistry, biotechnology, medicine and medical technology.

2. Background of the Related Art

Despite the fact that many cancers are considered to be sporadic diseases, changes in c-myc activity are frequently associated with a variety of human malignancies, e.g. cancer of the lung, breast, liver, colon, as well as ovarian cancer and lymphomas.

Lung cancer, for instance, remains the leading cause of cancer death worldwide. In 2007, approximately 160.000 people died from lung cancer in the United States alone (American Cancer Society, 2007). In Germany, lung cancer is the fourth-most frequent disease. More than 40,000 humans die at lung cancer annually (Federal Statistical Office, 2007) country wide and therefore it is one of the most frequent cancer disease at all. Smoking is considered the primary cause of lung cancer (relative risk >30-fold) and accounts for >80% of all diagnosed cases. Among other things, further risk factors are an exposition to radioactive compounds and the inhalation of heavy metals, asbestos and exhaust gases. In general, lung tumors are classified as small cell (SCLC) or non-small cell lung carcinomas (NSCLC). NSCLC are further divided into adenocarcinoma, large and squamous cell carcinoma. Subclasses of adenocarcinomas may be derived from Clara and alveolar epithelium. Primarily, lung cancer develops from the respiratory epithelium, whereby alveolar epithelial adenocarcinomas of humans are rather rare. Newer data point to a significant rise of alveolar and Clara cell tumors. Note, alveolar epithelial carcinomas may account for up to one third of all adenocarcinomas. The chances of survival of a patient with a lung adenocarcinoma are not even with 10% within 5 years.

The molecular mechanisms in the emergence of lung tumors are unclear and arise sporadically. Indeed, expression of c-myc is increased in many human malignant tumors resulting from an amplification of the reference allele or the mutated c-myc gene. Noteworthy, in the United States more than 70,000 cancer deaths per year are estimated to be related to c-myc abnormalities.

Since c-myc plays a key role in malignant tumor diseases, inhibition of c-myc expression may be sufficient to stop tumor growth permanently and to induce regression of tumors. However, as of today, disease regulated proteins in body fluids of patients suffering from c-myc-induced cancer, such as SCLC and NSCLC may be, are unknown.

### BRIEF SUMMARY OF THE INVENTION

There is a need for novel prognostic and predictive biomarkers and methods for easily identifying the indication “c-myc hyperactivity” or “c-myc overexpression,” respectively, or of aberrant signaling of the EGF receptor tyrosine kinase, in a subject suffering from or being susceptile to cancer, for predicting and monitoring the response of a subject to a treatment with c-myc activity modulators or with EGF receptor tyrosine kinase activity modulators, thereby enabeling an individualized cancer treatment of the subject for enhancing its chances of survival.

Therefore, one aim of the present invention is to provide biomarkers, compositions and a kit, as well as a method for a fast, easy and efficient qualification or quantification of the c-myc activity status of a subject suffering from or being susceptible to cancer, in particular for predicting and monitoring the response of a cancer patient to the treatment with a c-myc activity modulator or with an EGF receptor tyrosine kinase activity modulator.

It would be desirable to provide novel and enhanced means for diagnosis, prognosis and/or treatment monitoring of lung cancer or bronchial dysplasia.

### BRIEF DESCRIPTION OF THE FIGURES

FIG. 1a: Gene construct of the SP-C/c-myc transgenic mouse model as reported by Ehrhardt et al.

FIG. 1b: Histology of a lung of a healthy (A1) and a tumor bearing (B1) SP-C/c-myc transgenic mouse, aged 14 months. Hematoxylin and eosin staining is used for histopathology of tumors. A2 and B2: macroscopical views of lungs of healthy and tumor bearing mice, respectively.

FIG. 2a: 2-DE serum proteome map of lung tumor bearing SP-C/c-myc transgenic mice with a pH range from 3-10.

FIG. 2b: 2-DE serum proteome map of lung tumor bearing SP-C/c-myc transgenic mice with a pH range from 4-7.

FIG. 3a: Examples of regulated serum proteins in tumor bearing SP-C/c-myc transgenic (T) and healthy (C) mice. Protein spots of interest are marked by circles.

FIG. 3b: ppm values of 13 disease regulated proteins. A: control pH 4-7, B: tumor pH 4-7, C: control pH 3-10, D: tumor pH 3-10.

FIG. 4 shows a comparison of a number of biomarkers of the invention.

### DETAILED DESCRIPTION OF THE INVENTION

The basic finding, relevant to this invention, is the unexpected regulation of certain proteins in c-myc induced lung tumors.

By using two pH ranges (4-7 and 3-10) a mapping for the characterization of the serum proteome of the SP-C/c-myc mouse model is accomplished, which covers 46 identified proteins in total, 3 of which are unknown, yet. Moreover, 13 significantly disease regulated proteins are discovered, e.g. orosomucoid-8, alpha-2-macroglobulin, glutathione peroxidase 3, properdin, plasma retinol binding protein, transthyretin as well as numerous apolipoproteins, like apolipoprotein H, for which regularization in connection with lung cancer has not been reported, yet.

Furthermore, exclusively in tumor bearing mice a component of the serum amyloid P protein has been identified. These investigations provide new information about the mouse serum proteome and the role of c-myc in lung tumorigenesis as well. Therefore, serum proteomics leads to a routinely use of proteomic methods in clinical laboratories for the prognostic and diagnostic classification of several stages of the disease as well as for monitoring the evaluation of therapy processes.

Embodiments of the invention are based on the surprising finding that biomarkers selected from a first group consisting of Alpha-1-antitrypsin 1-1 (A1AT1), Alpha-l-antitrypsin 1-6 (A1AT6), Alpha-2-macroglobulin (A2MG), Properdin (PROP), Transthyretin (TTHY), Orosomucoid-8 (A1AG8), Apolipoprotein A-I (AP0A1), Apolipoprotein C-III (APOC3), Apolipoprotein H (APOH), Glutathione peroxidase 3 (GPX3), Plasma retinol-binding protein (RETBP), Serum amyloid P-component (SAMP), Vitamin D binding protein (VTDB), Sodium- and chloride-dependent GABA transporter 4 (S6A11), epidermal growth factor receptor (EGFR) or from a second group consisting of Apolipoprotein E (APOE) and fragments thereof are regulated by c-myc overexpression in subjects suffering from or being susceptile to cancer.

Moreover, it has been surprisingly found that biomarkers selected from the group of ApoA4, ApoM, a-raf, fetuin B, GSN, PLG, VPS28 are regulated by aberrant EGF receptor tyrosine kinase signaling in subjects having dysplasia or suffering from or being susceptile to cancer.

The biomarkers according to the invention concern gene products of mammalia, preferably gene products of the genome of mus musculus or homo sapiens, in particular the respective gene products of homo sapiens are preferred, or, respectively, sequence fragments of said gene products as described herein.

Within the context of the invention, the term “subject”, and “patient”, respectively, is directed to a mammal, in particular to a mouse or a human being suffering from or being susceptible to cancer, more particular to a human cancer patient or a transgenic cancer mouse, such as a SCLC or NSCLC patient or a c-myc-transgenic mouse or an EGF-transgenic mouse may be.

The term “dysplasia” according to the invention is directed to low grade and/or high grade dysplasia, wherein “low grade dysplasia” is particularly directed to a lesion having minimal aberration inside the cell, and “high grade dysplasia” also comprises mild or medium dysplasia. The term “bronchial dysplasia” according to the invention is in particular directed to lung dysplasia.

In one aspect, the invention is directed to the use, in particular to the in vitro use, of at least one biomarker selected from the group consisting of APOE, APOC3, A1AT6, A2MG, PROP, TTHY, A1AG8, APOA1, APOH, GPX3, MUP8, RETBP, SAMP, VTDB, S6A11, EGFR, ApoA4, ApoM, a-raf, fetuin B, GSN, PLG, VPS28, and/or to the use, in particular to the in vitro use, of at least one antibody directed against said at least one biomarker, in the diagnosis of cancer and/or the prognosis of cancer or dysplasia and/or the treatment monitoring of cancer or dysplaisa, in particular of lung cancer or bronchial dysplasia, preferably of lung adenocarcinoma(s).

Within the inventive context, antibodies are understood to include monoclonal antibodies and polyclonal antibodies and antibody fragments (e.g., Fab, and F(ab′)₂) specific for one of said polypeptides. Polyclonal antibodies against selected antigens may be readily generated by one of ordinary skill in the art from a variety of warm-blooded animals such as horses, cows, goats, rabbits, mice, rats, chicken or preferably of eggs derived from immunized chicken. Monoclonal antibodies may be generated using conventional techniques (see Monoclonal Antibodies, Hybridomas: A New Dimension in Biological Analyses, Plenum Press, Kennett, McKearn, and Bechtol (eds.), 1980, and Antibodies: A Laboratory Manual, Harlow and Lane (eds.), Cold Spring Harbor Laboratory Press, 1988, which are incorporated herein by reference).

Preferentially, a combination of APOE and at least one further biomarker selected from the group of APOC3, A1AT6, A2MG, PROP, TTHY, A1AG8, APOA1, APOH, GPX3, MUP8, RETBP, SAMP, VTDB, S6A11, EGFR is used as the at least one biomarker. Preferably, an appropriate amount of the at least one biomarker is used, in particular an amount for manufacturing a reference, more particular for manufacturing a reference comprising a reference level of said at least one biomarker, such as the level of said at least one biomarker in a sample of a normal healthy individual or the level of a said at least one biomarker in a sample of a patient suffering from lung cancer may be.

In particular, at least one biomarker selected from the group consisting of APOE, APOC3, A1AT6, A2MG, PROP, TTHY, A1AG8, APOA1, APOH, GPX3, MUP8, RETBP, SAMP, VTDB, S6A11, EGFR, ApoA4, ApoM, a-raf, fetuin B, GSN, PLG, VPS28, and/or at least one antibody directed against said at least biomarker, is used for monitoring the therapeutic treatment of a patient suffering from lung cancer or having bronchial dysplasia, in particular the treatment with a chemotherapeutic agent, preferably with an antineoplastic chemotherapy drug, or with a chemopreventive drug.

Preferably, the at least one biomarker selected from the group consisting of APOE, APOC3, A1AT6, A2MG, PROP, TTHY, A1AG8, APOA1, APOH, GPX3, MUP8, RETBP, SAMP, VTDB, S6A11, EGFR, ApoA4, ApoM, a-raf, fetuin B, GSN, PLG, VPS28, and/or at least one antibody directed against said at least biomarker, is used for monitoring the therapeutic treatment of a patient suffering from lung cancer with a chemotherapeutic drug usable against lung cancer, in particular the treatment with Paclitaxel, Gefitinib, Erlotinib, Etoposide, Carboplatin, Docetaxel, Vinorelbine tartrate, Cisplatin, Doxorubicin, Ifosfamide, Vincristine sul fate, Gemcitabine hydrochloride, Lomustine (CCNU), Cyclophosphamide, Methotrexate, Topotecan hydrochlorid, or with a combination thereof, or of a patient having bronchial dysplasia with a chemopreventive drug, such as Zileuton or Celecoxib may be.

In particular, it is preferred, if at least one biomarker selected from the group consisting of APOE, APOC3, A1AT6, A2MG, PROP, TTHY, A1AG8, APOA1, APOH, GPX3, MUP8, RETBP, SAMP, VTDB, S6A11, EGFR, ApoA4, ApoM, a-raf, fetuin B, GSN, PLG, VPS28, and/or at least one antibody directed against said at least biomarker is used, for monitoring the therapeutic treatment of a patient, in particular a human patient, suffering from lung cancer, in particular for monitoring the treatment of said patient with irinotecan, paclitaxel and/or 5-fluorouracil.

More preferably, the at least one biomarker used is selected from the group consisting of

APOE, APOC3, A1AG8, APOA1, APOH, GPX3, RETBP, SAMP, in particular for the diagnosis, prognosis and/or treatment monitoring of BAC.

Also, it may be preferred to use at least one biomarker selected from the group consisting of MUP8, VTDB, S6A11, EGFR, or at least one respective antibody, in particular for the diagnosis, prognosis and/or treatment monitoring of AAH.

Preferentially, a combination of at least one biomarker selected from the group consisting of APOE, APOC3, A1AG8, APOA1, APOH, GPX3, RETBP, SAMP and at least one biomarker selected from the group consisiting of MUP8, VTDB, S6A11, EGFR, or a respective combination of antibodies, is used.

Within the context of bronchial dysplasia and/or lung cancer (adenocarcinomas) it is preferred if the at least one biomarkeris selected from the group consisting of ApoA4, ApoM, a-raf, fetuin B, GSN, PLG, VPS28, in particular for the diagnosis, prognosis and/or treatment monitoring of bronchial dysplasia or lung cancer, wherein Fetuin B and/or PLG is particularly preferred in the context of bronchial dysplasia.

Another aspect of the invention is directed to a method, in particular an in vitro method, for diagnosing cancer or dysplaisa and/or prognosing cancer or dysplasia and/or staging cancer or dysplasia and/or monitoring the treatment of cancer or dysplasia, preferably of lung cancer or bronchial dysplasia, in particular of lung adenocarcinoma(s), comprising the steps of

(a) measuring the level of at least one biomarker selected from the group consisting of APOE, APOC3, A1AT6, A2MG, PROP, TTHY, A1AG8, APOA1, APOH, GPX3, MUP8, RETBP, SAMP, VTDB, S6A11, EGFR, ApoA4, ApoM, a-raf, fetuin B, GSN, PLG, VPS28 in a body fluid sample, in particular in a serum sample, in particular in a blood serum sample, of a patient suffering from or being susceptible to cancer, and

(b) comparing the level of said at least one biomarker in said sample to a reference level of said at least one biomarker, in particular by the use according to one of the claims 1-3.

Preferably, said method is used for monitoring the therapeutic treatment of a patient suffering from lung cancer or having bronchial dysplasia, in particular the treatment with a chemotherapeutic agent, preferably with an antineoplastic chemotherapy drug, or with a chemopreventive drug.

More preferably, said method is used for monitoring the therapeutic treatment of a patient suffering from lung cancer with a chemotherapeutic drug usable against lung cancer, in particular the treatment with Paclitaxel, Gefitinib, Erlotinib, Etoposide, Carboplatin, Docetaxel, Vinorelbine tartrate, Cisplatin, Doxorubicin, Ifosfamide, Vincristine sul fate, Gemcitabine hydrochloride, Lomustine (CCNU), Cyclophosphamide, Methotrexate, Topotecan hydrochlorid, or with a combination thereof, or of a patient having bronchial dysplasia with a chemopreventive drug, such as Zileuton or Celecoxib may be.

In particular, it is preferred, to implement said method for monitoring the therapeutic treatment of a patient, in particular a human patient, suffering from lung cancer, in particular for monitoring the treatment of said patient with irinotecan, paclitaxel and/or 5-fluorouracil.

Preferably, the method for diagnosing, prognosing and/or staging cancer and/or monitoring the treatment of cancer, is implemented for monitoring the therapeutic treatment of a patient suffering from lung cancer, in particular the treatment with irinotecan, paclitaxel and/or 5-fluorouracil.

In one preferred embodiment, at least one biomarker is selected from the group consisting of APOE, APOC3, A1 AG8, APOA1, APOH, GPX3, RETBP, SAMP, in particular for the diagnosis, prognosis, staging and/or treatment monitoring of BAC.

In another preferred embodiment at least one biomarker is selected from the group consisting of MUP8, VTDB, S6A11, EGFR, in particular for the diagnosis, prognosis, staging and/or treatment monitoring of AAH.

In a further preferred embodiment, at least one biomarker is selected from the group consisting of ApoA4, ApoM, a-raf, fetuin B, GSN, PLG, VPS28, in particular for the diagnosis, prognosis, staging and/or treatment monitoring of dysplasia or cancer, in particular of bronchial dysplasia or lung cancer, preferably of adenocarcinoma(s), wherein fetuin B and/or PLG is/are in particular preferred in the context of bronchial dysplasia.

It is thus understood, that according to the invention preferably fetuin B or PLG or a combination thereof, or fragments of fetuin B or PLG or a combination thereof, or antibodies directed against fetuin B and/or PLG and/or their fragements, are used within the context of diagnosing or treatment monitoring of dysplasia.

In particular, the method is preferably implemented to distinguish between different subtypes of lung cancer, such as (but not limited to) lung adenocarcinomas as defined by AAH or BAC, wherein at least one biomarker selected from the group consiting of APOE, APOC3, A1AG8, APOA1, APOH, GPX3, RETBP, SAMP and at least one biomarker selected from the group consisiting of MUP8, VTDB, S6A11, EGFR are measured, and, more particularly, wherein a significantly altered level of APOE, APOC3, A1AG8, APOA1, APOH, GPX3, RETBP and/or SAMP in comparison with the respective level of a normal individual is indicative of BAC and wherein a significantly altered level of MUP8, VTDB, S6A1 1 and/or EGFR in comparison with the respective level of a normal individual is indicative of AAH.

In another aspect, the invention further concerns a composition for qualifying the c-myc activity in a subject suffering from or being susceptible to cancer, in particular by an in vitro body fluid analysis, wherein the composition comprises an effective amount of at least one biomarker selected from the first group of said biomarkers or an effective amount of at least one biomarker selected from the second group of said biomarkers, and/or comprises at least one antibody directed against said at least one biomarker, in particular for use in qualifying the c-myc activity in a patient suffering or being susceptible to cancer or for use in classifying a patient suffering from or being susceptible to lung cancer.

In one embodiment of the invention, the biomarker is preferably selected from a first group consisting of A1AG8, APOA1, APOC3, APOH, GPX3, RETBP, SAMP or from a second group consisting of APOE and fragments thereof.

In another preferred embodiment, a biomarker is provided for qualifying the c-myc activity in a patient suffering or being susceptible to cancer or for classifying a patient suffering from or being susceptible to lung cancer, wherein the biomarker is selected from a first group consisting of sequence fragments of the first group of said biomarkers or is selected from a second group consisting of sequence fragments of the second group of said biomarkers, and wherein the sequence fragments are 6-24 amino acid residues in length and are preferably synthetic peptides.

Also a biomarker, preferably for use in diagnosing or treatment monitoring of dysplasia or lung cancer, in particular bronchial dysplasia or lung cancer, is provided, wherein the biomarker is selected from the group consisiting of ApoA4, ApoM, a-raf, fetuin B, GSN, PLG, VPS28, wherein the biomarker is regulated by aberrant EGF receptor tyrosine kinase signaling in a subject, and/or an antibody directed against said biomarker is provided, preferably for use in diagnosing or treatment monitoring of lung cancer, in particular bronchial dysplasia or lung cancer.

In a further preferred embodiment a biomarker, preferably for use in diagnosing or treatment monitoring of dysplasia or cancer, in particular of bronchial dysplasia or lung cancer, associated with aberrant EGF receptor tyrosine kinase signaling in a patient, is provided, selected from a group consisting of sequence fragments of the group of ApoA4, ApoM, a-raf, fetuin B, GSN, PLG, VPS28, wherein the sequence fragments are 6-24 amino acid residues in length and are preferably synthetic peptides.

Preferably, the biomarker is selected from a first group consisting of LAQIHFPR (SEQ ID No: 1), TLMSPLGITR (SEQ ID No: 2), RLAQIHFPR (SEQ ID NO: 3), MQHLEQTLSK (SEQ ID NO: 4), ELISKFLLNR (SEQ ID NO: 5), IFNNGADLSGITEENAPLK (SEQ ID NO: 6), NHYQAEVFSVNFAESEEAK (SEQ ID NO: 7), DQSPASHEIATNLGDFAISLYR (SEQ ID NO: 8), KPFDPENTEEAEFHVDESTTVK (SEQ ID NO: 9), FDHPFLFIIFEEHTQSPLFVGK (SEQ ID NO: 10), APFALQVNTLPLNFDK (SEQ ID NO: 11), TCDHPAPR (SEQ ID NO: 12), QRLCTPLLPK (SEQ ID NO: 13), HGGPFCAGDATR (SEQ ID NO: 14), MSINCEGTPGQQSR (SEQ ID NO: 15), LRMSINCEGTPGQQSR (SEQ ID NO: 16), HGGPFCAGDATRNQMCNK (SEQ ID NO: 17), CGGHCPGEAQQSQACDTQK (SEQ ID NO: 18), SCSAPAPSHQPPGKPCSGPAYEHK (SEQ ID NO: 19), FVEGVYR (SEQ ID NO: 20), TSEGSWEPFASGK (SEQ ID NO: 21), TAESGELHGLTTDEK (SEQ ID NO: 22), TLGISPFHEFADVVFTANDSGHR (SEQ ID NO: 23), HYTIAALLSPYSYSTTAVVSNPQN (SEQ ID NO: 24), YEGGVETFAHLIVLR (SEQ ID NO: 25), LQELQGR (SEQ ID NO: 26), EDVELYR (SEQ ID NO: 27), ARPALEDLR (SEQ ID NO: 28), LSPVAEEFR (SEQ ID NO: 29), QKLQELQGR (SEQ ID NO: 30), WKEDVELYR (SEQ ID NO: 31), VQPYLDEFQK (SEQ ID NO: 32), TQLAPHSEQMR (SEQ ID NO: 33), LSPVAEEFRDR (SEQ ID NO: 34), SNPTLNEYHTR (SEQ ID NO: 35), VAPLGAELQESAR (SEQ ID NO: 36), QEMNKDLEEVK (SEQ ID NO: 37), VKDFANVYVDAVK (SEQ ID NO: 38), LQELQGRLSPVAEEFR (SEQ ID NO: 39), TVQDALSSVQESDIAVVAR (SEQ ID NO: 40), IHFYCK (SEQ ID NO: 41), ATVLYQGMR (SEQ ID NO: 42), ITCPPPPVPK (SEQ ID NO: 43), WSPDIPACAR (SEQ ID NO: 44), DGTIEIPSCFK (SEQ ID NO: 45), CSYTVEAHCR (SEQ ID NO: 46), TGTWSFLPTCR (SEQ ID NO: 47), VCPFAGILENGIVR (SEQ ID NO: 48), IQEQFKNGMMHGDK (SEQ ID NO: 49), FTCPLTGMWPINTLR (SEQ ID NO: 50), ICPKPDDLPFATVVPLK (SEQ ID NO: 51), TSYDPGEQIVYSCKPGYVSR (SEQ ID NO: 52), CPFPPRPENGYVNYPAKPVLLYK (SEQ ID NO: 53), YVRPGGGFVPNFQLFEK (SEQ ID NO: 54), DPNGLSPETR (SEQ ID NO: 55), YWGVASFLQR (SEQ ID NO: 56), QRQEELCLER (SEQ ID NO: 57), LQNLDGTCADSYSFVFSR (SEQ ID NO: 58), KDPEGLFLQDNIIAEFSVDEK (SEQ ID NO: 59), APPSIVLGQEQDNYGGGFQR (SEQ ID NO: 60), or from a second group consisting of EVQAAQAR (SEQ ID NO: 61), FWDYLR (SEQ ID NO: 62), DRLEEVR (SEQ ID NO: 63), EHMEEVR (SEQ ID NO: 64), LGPLVEQGR (SEQ ID NO: 65), LEEVGNQAR (SEQ ID NO: 66), QWANLMEK (SEQ ID NO: 67), DRAQAFGDR (SEQ ID NO: 68), LQAEIFQAR (SEQ ID NO: 69), MEEQTQQIR (SEQ ID NO: 70), GRLEEVGNQAR (SEQ ID NO: 71), TANLGAGAAQPLR (SEQ ID NO: 72), SKMEEQTQQIR (SEQ ID NO: 73), GWFEPIVEDMHR (SEQ ID NO: 74), ELEEQLGPVAEETR (SEQ ID NO: 75), NEVHTMLGQSTEEIR (SEQ ID NO: 76), or from a third group, consisting of GNTEGLQK (SEQ ID NO: 116), LQLTPYIQR (SEQ ID NO: 117), ALVQQLEQFR (SEQ ID NO: 118), QLEQQVEEFR (SEQ ID NO: 119), ATIDQNLEDLRR (SEQ ID NO: 120), QLEQQVEEFRR (SEQ ID NO: 121), LNHQMEGLAFQMK (SEQ ID NO: 122), TDVTQQLSTLFQDK (SEQ ID NO: 123), LVPFVVQLSGHLAKETER (SEQ ID NO: 124), QQLGPNSGEVESHLSFLEK (SEQ ID NO: 125), LQEHLKPYAVDLQDQINTQTQEMK (SEQ ID NO: 126), LVPFVVQLSGHLAKETER (SEQ ID NO: 127), AFLVTPR (SEQ ID NO: 128), FLLYNR (SEQ ID NO: 129), ETGQGYQR (SEQ ID NO:130), SPHSKLPSEQR (SEQ ID NO: 131), LVVLPFPGK (SEQ ID NO: 132), DGYMLSLNR (SEQ ID NO: 133), AMFHINKPR (SEQ ID NO: 134), AMNQWVSGPAYYVEYLIK (SEQ ID NO: 135), SQASCSLQHSDSEPVGICQGSTVQSSLR (SEQ ID NO: 136), EHYQEDMGSLFYLTLDVLETDCHVLSR (SEQ ID NO: 137), LFACSNR (SEQ ID NO: 138), RTPITVVR (SEQ ID NO: 139), EPGLQIWR (SEQ ID NO: 140) DGGQTAPASIR (SEQ ID NO: 141), AGKEPGLQIWR (SEQ ID NO: 142), HVVPNEVVVQR (SEQ ID NO: 143), SEDCFILDHGR (SEQ ID NO: 144), EVQGFESSTFSGYFK (SEQ ID NO: 145) QTQVSVLPEGGETPLFK (SEQ ID NO: 146), EPAHLMSLFGGKPMIIYK (SEQ ID NO: 147), SQHVQVEEGSEPDAFWEALGGK (SEQ ID NO: 148), VSNGAGSMSVSLVADENPFAQGALR (SEQ ID NO: 149) VPVDPATYGQFYGGDSYIILYNYR (SEQ ID NO: 150), NWRDPDQTDGPGLGYLSSHIANVER (SEQ ID NO: 151)IEGSNKVPVDPATYGQFYGGDSYIILYNYR (SEQ ID NO: 152), HVVPNEVVVQR (SEQ ID NO: 153), FVDWIER (SEQ ID NO: 154), LILEPNNR (SEQ ID NO: 155), SSRPEFYK (SEQ ID NO: 156), WSEQTPHR (SEQ ID NO: 157), NLEENYCR (SEQ ID NO: 158), WEYCDIPR (SEQ ID NO: 159), MRDVILFEK (SEQ ID NO: 160), WEYCNLKR (SEQ ID NO: 161), CEGETDFVCR (SEQ ID NO: 162), HSIFTPQTNPR (SEQ ID NO: 163), VILGAHEEYIR (SEQ ID NO: 164), CQSWAAMFPHR (SEQ ID NO: 165), VCNRVEYLNNR (SEQ ID NO: 166), GPWCYTTDPSVR (SEQ ID NO: 167), GTVSVTVSGKTCQR (SEQ ID NO: 168), DIALLKLSRPATITDK (SEQ ID NO: 169), VIPACLPSPNYMVADR (SEQ ID NO: 170), CTTPPPPPSPTYQCLK (SEQ ID NO: 171), TPENFPDAGLEMNYCR (SEQ ID NO: 172), NPDGDVNGPWCYTTNPR (SEQ ID NO: 173), NPDGEPRPWCFTTDPTK (SEQ ID NO: 174), NPDGDKGPWCYTTDPSVR (SEQ ID NO: 175), TICYITGWGETQGTFGAGR (SEQ ID NO: 176), TAVTAAGTPCQGWAAQEPHR (SEQ ID NO: 177), NPDGETAPWCYTTDSQLR (SEQ ID NO: 178), VVGGCVANPHSWPWQISLR (SEQ ID NO: 179), NPDGEPRPWCFTTDPTKR (SEQ ID NO: 180), NPDNDEQGPWCYTTDPDKR (SEQ ID NO: 181), YILQGVTSWGLGCARPNKPGVYVR (SEQ ID NO: 182), FTGQHFCGGTLIAPEWVLTAAHCLEK (SEQ ID NO: 183), WGATFPHVPNYSPSTHPNEGLEENYCR (SEQ ID NO: 184), LEIRAMDEIQPDLR (SEQ ID NO: 185), LEIRAMDEIQPDLR (SEQ ID NO: 186), QVQGSEISSIDEFCRK (SEQ ID NO: 187), YDNMAELFAVVKTMQALEK (SEQ ID NO: 188) , AMDEIQPDLRELMETMHR (SEQ ID NO: 189), QTVSQWLQTLSGMSASDELDDSQVR (SEQ ID NO: 190), and FHGIPATPGVGAPGNKPELYEEVKLYK (SEQ ID NO: 191)

and wherein in the peptide(s) according to three groups, if applicable, at least one amino acid methionine is preferably oxidized according to the Supplementary tables 1.

More preferably, the biomarker is selected from a first group consisting of

YEGGVETFAHLIVLR (SEQ ID NO: 77), LQELQGR (SEQ ID NO: 78), EDVELYR

(SEQ ID NO: 79), ARPALEDLR (SEQ ID NO: 80), LSPVAEEFR (SEQ ID NO: 81), QKLQELQGR (SEQ ID NO: 82), WKEDVELYR (SEQ ID NO: 83), VQPYLDEFQK (SEQ ID NO: 84), TQLAPHSEQMR (SEQ ID NO: 85), LSPVAEEFRDR (SEQ ID NO: 86), SNPTLNEYHTR (SEQ ID NO: 87), VAPLGAELQESAR (SEQ ID NO: 88), QEMNKDLEEVK (SEQ ID NO: 89), VKDFANVYVDAVK (SEQ ID NO: 90), LQELQGRLSPVAEEFR (SEQ ID NO: 91), TVQDALSSVQESDIAVVAR (SEQ ID NO: 92), IHFYCK (SEQ ID NO: 93), ATVLYQGMR (SEQ ID NO: 94), ITCPPPPVPK (SEQ ID NO: 95), WSPDIPACAR (SEQ ID NO: 96), DGTIEIPSCFK (SEQ ID NO: 97), CSYTVEAHCR (SEQ ID NO: 98), TGTWSFLPTCR (SEQ ID NO: 99), VCPFAGILENGIVR (SEQ ID NO: 100), IQEQFKNGMMHGDK (SEQ ID NO: 101), FTCPLTGMWPINTLR (SEQ ID NO: 102), ICPKPDDLPFATVVPLK (SEQ ID NO: 103), TSYDPGEQIVYSCKPGYVSR (SEQ ID NO: 104), CPFPPRPENGYVNYPAKPVLLYK (SEQ ID NO: 105), YVRPGGGFVPNFQLFEK (SEQ ID NO: 106), IEDNGNFR (SEQ ID NO: 107), EKIEDNGNFR (SEQ ID NO: 108), ENIIDLSNANR (SEQ ID NO: 109), FAQLCEEHGILR (SEQ ID NO: 110), DGETFQLMGLYGR (SEQ ID NO: 111), INGEWHTIILASDKR (SEQ ID NO: 112), TDYDNFLMAHLINEK (SEQ ID NO: 113), LFLEQIHVLENSLVLK (SEQ ID NO: 114), AGEYSVTYDGFNTFTIPK (SEQ ID NO: 115), DPNGLSPETR (SEQ ID NO: 55), YWGVASFLQR (SEQ ID NO: 56), QRQEELCLER (SEQ ID NO: 57), LQNLDGTCADSYSFVFSR (SEQ ID NO: 58), KDPEGLFLQDNIIAEFSVDEK (SEQ ID NO: 59), APPSIVLGQEQDNYGGGFQR (SEQ ID NO: 60), or from a second group consisting of EVQAAQAR (SEQ ID NO: 61), FWDYLR (SEQ ID NO: 62), DRLEEVR (SEQ ID NO: 63), EHMEEVR (SEQ ID NO: 64), LGPLVEQGR (SEQ ID NO: 65), LEEVGNQAR (SEQ ID NO: 66), QWANLMEK (SEQ ID NO: 67), DRAQAFGDR (SEQ ID NO: 68), LQAEIFQAR (SEQ ID NO: 69), MEEQTQQIR (SEQ ID NO: 70), GRLEEVGNQAR (SEQ ID NO: 71), TANLGAGAAQPLR (SEQ ID NO: 72), SKMEEQTQQIR (SEQ ID NO: 73), GWFEPIVEDMHR (SEQ ID NO: 74), ELEEQLGPVAEETR (SEQ ID NO: 75), NEVHTMLGQSTEEIR (SEQ ID NO: 76), and wherein in the peptide(s) according to two groups, if applicable, at least one amino acid methionine is preferably oxidized according to the Supplementary tables 1.

In particular, it is preferred, if the composition according to the invention, which is in particular a composition for use in qualifying the c-myc activity in a patient suffering from or being susceptible to cancer or for use in classifying a patient suffering from or being susceptible to lung cancer, comprises an effective amount of at least one biomarker selected from the first group of said biomarkers and an effective amount of at least one biomarker selected from the second group of said biomarkers, or comprises an effective amount of at least one antibody directed against said at least one biomarker, wherein the combination biomarker selected from the group consisting of A1AT1, A1AT6, A2MG, PROP, TTHY, A1AG8, APOA1, APOC3, APOH, GPX3, RETBP, SAMP or selected from the group consisting of the sequence fragments thereof, as described herein, and biomarker selected from the group consisting of APOE or selected from the group of the sequence fragments thereof, as described herein, or the combination biomarker selected from the group consisting of A1AG8, APOA1, APOC3, APOH, GPX3, RETBP, SAMP, AFP, ApoE, ApoM or selected from the group consisting of the sequence fragments thereof, as described herein, and biomarker selected from the group consisting of APOE or selected from the group consisting of the sequence fragments thereof, as described herein, is particularly preferred.

In another preferred embodiment said composition further comprises an effective amount of a biomarker selected from the group of c-myc, thus allowing an easy calibration of the system.

In another preferred embodiment, the composition according to the invention, which is in particular a composition for use in diagnosing or treatment monitoring of dysplasia or cancer, in particular of bronchial dysplasia or lung cancer, associated with aberrant EGF receptor tyrosine kinase signaling in a patient, preferably by an in vitro body fluid analysis, comprises an effective amount of at least one biomarker selected from the group of ApoA4, ApoM, a-raf, fetuin B, GSN, PLG, VPS28, or comprises an effective amount of at least one antibody directed against said at least one biomarker, or comprises an effective amount of at least one sequence fragment of said at least on biomarker, in particular of the third group of fragments, as described herein.

In another preferred embodiment said composition further comprises an effective amount of a biomarker selected from the group of EGF, thus allowing an easy calibration of the system.

In yet another preferred embodiment, the composition according to the invention further comprises an effective amount of a protease, in particular of trypsin, thus enabling a further enhancement of the system sensitivity.

The composition according to the invention, in particular the protease digest thereof, may be preferably used for producing a vaccine for the immunization of an animal in order to produce polyclonal antibodies specific for the at least one biomarker.

Another aspect of the invention concerns the use of the composition according to the invention for the production of a diagnostic agent, in particular of a diagnostic standard for in vitro body fluid analyses.

The term “body fluid” according to the invention is directed to any body fluid of a subject, in particular to blood, plasma, serum or urine, whereas blood serum is the preferred body fluid within the context of the invention.

The term “diagnostic agent” as used herein relates to any solution, suspension or solid formulation, containing said composition in an acceptable amount for diagnostic purposes.

In particular, the composition is used for the production of a diagnostic agent for qualifying the c-myc activity in a subject suffering from or being susceptible to cancer, preferably cancer of the lung, breast, liver, colon, as well as ovarian cancer and lymphomas, in particular in a subject suffering from or being susceptible to SCLC or NSCLC.

In a further preferred embodiment, the composition according to the invention is used for the production of a diagnostic agent for predicting or monitoring the response of a cancer patient to a method of treating cancer comprising administering a c-myc activity modulator to the patient.

In another embodiment the composition comprising an effective amount of at least one biomarker selected from the group of ApoA4, ApoM, a-raf, fetuin B, GSN, PLG, VPS28, or comprising an effective amount of at least one antibody directed against said at least one biomarker, or comprising an effective amount of at least one sequence fragment of said at least on biomarker, in particular of the third group of fragments, as described herein, is used for the production of a diagnostic agent, in particular of a diagnostic standard for body fluid analysis, preferably for predicting or monitoring the response of a dysplasia or cancer patient, in particular having bronchial dysplasia or lung cancer, to a method of treating dysplasia by administering a chemopreventive drug, such as Zileuton or Celecoxib may be or to a method treating cancer comprising administering an EGF receptor tyrosine kinase activity modulator, such as Gefitinib and/or Erlotinib.

In yet another aspect, the invention provides a kit for qualifying the c-myc activity in a subject suffering from or being susceptible to cancer, in particular for predicting or monitoring the response of a cancer patient to a method of treating cancer comprising administering an c-myc activity modulator, wherein the kit comprises at least one standard (1) indicative of the body fluid level of a biomarker selected from the first group of said biomarkers in normal individuals or individuals having cancer associated with increased c-myc activity and/or at least one standard (2) indicative of the body fluid level of a biomarker selected from the second group of said biomarkers in normal individuals or individuals having cancer associated with increased c-myc activity, and/or comprises at least one antibody directed against said at least one biomarker, in particular for use in qualifying the c-myc activity in a patient suffering or being susceptible to cancer or for use in classifying a patient suffering from or being susceptible to lung cancer, and instructions for the use of the kit.

In a preferred embodiment of the kit, the standard (1) comprises an indicative amount of at least one biomarker selected from the first group of said biomarkers and/or the at least one standard (2) comprises an indicative amount of at least one biomarker selected from the second group of said biomarkers.

In another preferred embodiment, the kit comprises a mixture of the at least one standard (1) and the at least one standard (2), in particular a composition according to the invention comprising an effective amount of at least one biomarker selected from the first group of said biomarkers and an effective amount of at least one biomarker selected from the second group of said biomarkers, wherein the set of biomarkers according to combination (a) or combination (b), as described herein, is particularly preferred.

In a further preferred embodiment a kit for use in diagnosing or treatment monitoring of dysplasia or cancer, in particular of bronchial dysplasia or lung cancer, associated with aberrant EGF receptor tyrosine kinase signaling in a patient, in particular for use in predicting or monitoring the response of the dysplasia patient or the cancer patient to a method of treating dysplasia by administering a chemopreventive drug, such as Zileuton or Celecoxib may be, or to a method of treating cancer by administering an EGF receptor tyrosine kinase activity modulator, such as Gefitinib and/or Erlotinib may be, is provided, wherein the kit comprises at least one standard indicative of the body fluid level of a biomarker selected from the group according to one of the claim 8 b) or 8 d) or according to the third group according to claim 9 in normal individuals or individuals having dysplasia or cancer related to aberrant EGF receptor tyrosine kinase signaling, and/or comprises at least one antibody directed against said at least one biomarker, and instructions for the use of the kit, and, preferably, reagents effective to detect said biomarker(s) in a serum sample, such as buffers for dissolving or equilibrating the standard and/or the antibodies, or an enzyme substrate for imaging enzyme labels may be.

In yet another preferred embodiment, the kit according to the invention further comprises a lysis buffer, wherein the lysis buffer comprises (a) at least one buffer component, (b) at least one chaotrope, (c) at least one detergens, (d) at least one reducing agent (e) at least one carrier ampholyte, and (f) at least one ribonuclease, Preferably, the lysis buffer is an aqueous solution of (a) at least one buffer compound selected from the group consisting of Tris and HEPES, (b) at least one chaotrope selected from the group consisting of urea and thiourea, (c) at least one detergens selected from the group consisting of CHAPS and SDS, (d) at least one reducing agent selected from the group consisting of DTT and TCEP, (e) at least one carrier ampholyte selected from the group consisting of biolyte 5-7 and biolyte 3-10, and (f) at least one ribonuclease selected from the group consisting of endonuclease and exonuclease, wherein an aqueous solution of (a) Tris; (b) urea and thiourea, (c) CHAPS, (d) DTT, (e) biolyte 3-10, and (f) endonuclease, is particularly preferred.

In one preferred embodiment, the kit according to the invention further comprises at least one antibody specific for a biomarker selected from the first group of said biomarkers and/or at least one antibody specific for a biomarker selected from the second group of said biomarkers, and reagents effective to detect said biomarker(s) in a serum sample, such as buffers for dissolving or equilibrating the standard (1) and/or the standard (2), or an enzyme substrate for imaging enzyme labels may be.

In particular, a kit is preferred, comprising at least one antibody specific for a biomarker selected from the group consisting of A1AT1, A1AT6, A2MG, PROP, TTHY, A1AG8, APOA1, APOC3, APOH, GPX3, RETBP, SAMP or selected from the group consisting of the sequence fragments thereof, as described herein, and/or at least one antibody specific for a biomarker selected from the group consisting of APOE or selected from the group consisting of the sequence fragments thereof, as described herein, or a peptide fragment thereof, as described herein.

More particular, it is preferred, if the at least one antibody is polyclonal, thus allowing a further enhancement of the system sensitivity. Advantageously, the kit further comprises at least one labelled secondary antibody specific for the at least one antibody, thus allowing a fast screening of the binding of the at least one antibody to the at least one biomarker, in particular if the at least one biomarker or the digest thereof is immobilized to a solid phase support, such as nitrocellulose may be.

In a further aspect, the invention provides a method of qualifying the c-myc activity in a subject, comprising determining in a body fluid sample of a subject suffering from or being susceptible to cancer at least one biomarker selected from the first group of said biomarkers and/or at least one biomarker selected from the second group of said biomarkers, wherein the body fluid level of the at least one biomarker of said first group being significantly higher and/or the body fluid level of the at least one biomarker of said second group being significantly lower than the level of said biomarker(s) in the body fluid of subjects without cancer, in particular without cancer associated with increased activity of c-myc, is indicative of induced c-myc activity in the subject.

In particular, it is preferred, if the method comprises determining at least one biomarker selected from the first group of said biomarkers and at least one biomarker selected from the second group of said biomarkers, wherein the body fluid level of the at least one biomarker of said first group being significantly higher and the body fluid level of the at least one biomarker of said second group being significantly lower than the level of said biomarkers in the body fluid of subjects without cancer, in particular without cancer associated with increased activity of c-myc, is indicative of induced c-myc activity in the subject, preferably if a combination of a biomarker selected from the group consisting of A1AT1, A1AT6, A2MG, PROP, TTHY, A1AG8, APOA1, APOC3, APOH, GPX3, RETBP, SAMP or selected from the group consisting of the sequence fragments thereof, as described herein, and a biomarker selected from the group consisting of APOE or selected from the group of the sequence fragments thereof, as described herein, is determined.

Preferably, the method according to the invention is carried out for predicting the response of a cancer patient to a method of treating cancer comprising administering an c-myc activity modulator, wherein the body fluid level of the at least one biomarker of said first group being significantly higher and/or the body fluid level of the at least one biomarker of said second group being significantly lower than the level of said biomarker(s) in the body fluid of subjects without cancer, in particular without cancer associated with increased activity of c-myc, is indicative that the subject will respond therapeutically to a method of treating cancer comprising administering a c-myc activity modulator.

In one embodiment, the method is implemented for monitoring the therapeutically response of a cancer patient to a method of treating cancer comprising administering a c-myc activity, wherein the body fluid level of the at least one biomarker of said first group before and after the treatment and/or the body fluid level of the at least one biomarker of said second group before and after the treatment is determined, and a significant decrease of said body fluid level(s) of the at least one biomarker of said first group and/or a significant increase of said body fluid level(s) of the at least one biomarker of said second group after the treatment is indicative that the cancer patient therapeutically responds to the administration of the c-myc activity modulator.

Also, the invention is directed to a method of diagnosing or treatment monitoring of dysplasia or cancer, in particular of bronchial dysplasia or lung cancer, associated with aberrant EGF receptor tyrosine kinase signaling in a patient, in particular for predicting or monitoring the response of the dysplasia patient or the cancer patient to a treatment of dysplasia by administering a chemopreventive drug, such as Zileuton or Celecoxib may be, or to a treatment of cancer by administering an EGF receptor tyrosine kinase activity modulator, such as Gefitinib and/or Erlotinib may be, wherein the method comprises determining in a body fluid sample of a subject having or being susceptible to dysplasia at least one biomarker selected from the group of fetuin B, GSN, VPS28 or their fragments according to one of the claims 8 b), 8 d), 9 and/or at least one biomarker selected from the group of ApoA4, ApoM, a-raf, PLG or their fragments according to one of the claims 8 b), 8 d), 9 wherein the body fluid level of the at least one biomarker of the group of fetuin B, GSN, VPS28 or their fragments being significantly higher and/or the body fluid level of the at least one biomarker related to the group of ApoA4, ApoM, a-raf, PLG or their fragments being significantly lower than the level of said biomarker(s) in the body fluid of subjects without dysplasia or cancer associated with aberrant EGF receptor tyrosine kinase signaling, is indicative of aberrant EGF receptor tyrosine kinase signaling in the subject.

In a preferred embodiment, said method is used for predicting the response of a dysplasia patient to a treatment of dysplasia comprising administering a chemopreventive drug, such as Zileuton or Celecoxib may be, or of a cancer patient to a treatment of cancer comprising administering an EGF receptor tyrosine kinase activity modulator, such as Gefitinib and/or Erlotinib may be, wherein the body fluid level of the at least one biomarker of the group of fetuin B, GSN, VPS28 or their fragments being significantly higher and/or the body fluid level of the at least one biomarker of the group of ApoA4, ApoM, a-raf, PLG or their fragments being significantly lower than the level of said biomarker(s) in the body fluid of subjects without dysplasia or cancer associated with aberrant EGF receptor tyrosine kinase signaling is indicative that the subject will respond therapeutically to a method of treating dysplasia by administering a chemopreventive drug, such as Zileuton or Celecoxib may be, or to a method of treating cancer by administering an EGF receptor tyrosine kinase activity modulator, such as Gefitinib and/or Erlotinib may be.

In a particular preferred embodiment, said method is used for monitoring the therapeutically response of a dysplasia patient to a treatment of dysplasia comprising administering a chemopreventive drug, or of a cancer patient to a treatment of cancer comprising administering an EGF receptor tyrosine kinase activity modulator, wherein the body fluid level of the at least one biomarker selected from the group of fetuin B, GSN, VPS28 or their fragments before and after the treatment and/or the body fluid level of the at least one biomarker selected from the group of ApoA4, ApoM, a-raf, PLG or their fragments before and after the treatment is determined, and a significant decrease of said body fluid level(s) of the at least one biomarker selected from the group of fetuin B, GSN, VPS28 or their fragments and/or a significant increase of said body fluid level(s) of the at least one biomarker selected from the group of ApoA4, ApoM, a-raf, PLG or their fragments after the treatment is indicative that the dysplasia patient therapeutically responds to the administration of a chemopreventive drug, or that the cancer patient therapeutically responds to the EGF receptor tyrosine kinase activity modulator.

In a preferred embodiment, the method is implemented by performing an immunoassay, such as an enzyme immunoassay (EIA), a radio immunoassay (RIA) or a fluorescence immunoassay (FIA) may be, in particular by using the kit according to the invention and/or by performing a western blot. Preferably, at least one antibody specific for a biomarker selected from the group consisting of A1AT1, A1AT6, A2MG, PROP, TTHY, A1AG8, APOA1, APOC3, APOH, GPX3, RETBP, SAMP or selected from the group consisting of the sequence fragments thereof, as described herein, and/or at least one antibody specific for a biomarker selected from the group consisting of APOE or selected from the group consisting of the sequence fragments thereof, as described herein, and/or at least one antibody specific for a biomarker selected from the group consisting of ApoA4, ApoM, a-raf, fetuin B, GSN, PLG, VPS28, or selected from the group consisting of the sequence fragments thereof, as described herein, is used for the immunoassay and/or reagents effective to detect said biomarker(s) in a serum sample, such as a blocking buffer for reducing secondary antibodies unspecific antibody binding or an enzyme substrate for imaging enzyme labelled antibodies may be, is used for the immunoassay.

Within the context of immunoassys, the method preferably comprises the steps of isolating a serum sample from a blood sample of a subject suffering from or being susceptible to cancer,

adding lysis buffer to the serum sample;

separating the proteins of the lysed serum sample by 2-D gel electrophoresis;

excising from the gel at least one sample containing a protein of interest;

adding digesting buffer to the at least one excised sample, and

determining the amount of the at least one protein of interest by analyzing the at least one digest mixture by an immunoassay, in particular by a Western blot.

In another preferred embodiment, the method is implemented by performing a peptide mass fingerprinting and/or a peptide fragmentation fingerprinting, in particular by using the kit described herein.

Within the context of peptide mass fingerprinting, the method preferably comprises the steps of

isolating a serum sample from a blood sample of a subject suffering from or being susceptible to cancer,

adding lysis buffer to the serum sample;

separating the proteins of the lysed serum sample by 2-D gel electrophoresis;

excising from the gel at least one sample containing a protein of interest;

adding digesting buffer to the at least one excised sample, and

determining the amount of the at least one protein of interest by analyzing the at least one digest mixture by mass spectrometry.

In one embodiment of the method, the subject is a human patient or a non-human transgenic animal, in particular suffering from or being susceptible to cancer or having or being susceptible to dysplasia, particularly bronchial dysplasia, more particular suffering from or being susceptible to cancer of the lung, breast, liver, colon, as well as ovarian cancer and lymphomas, such as a transgenic mouse, in particular a mouse whose genome comprises a non natural c-myc sequence or a non natural EGF sequence, may be.

In another embodiment of the method, the serum sample is isolated by centrifuging the blood sample.

In yet another embodiment of the method, the 2-DE is performed by using two different pH gradients, preferably by using the pH gradients 3-10 and 4-7.

In a further embodiment of the method, the lysis buffer comprises (a) at least one buffer component, (b) at least one chaotrope, (c) at least one detergens, (d) at least one reducing agent (e) at least one carrier ampholyte, and (f) at least one ribonuclease. Preferably, the lysis buffer used is an aqueous solution of (a) at least one buffer compound selected from the group consisting of Tris and HEPES, (b) at least one chaotrope selected from the group consisting of urea and thiourea, (c) at least one detergens selected from the group consisting of CHAPS and SDS, (d) at least one reducing agent selected from the group consisting of DTT and TCEP, (e) at least one carrier ampholyte selected from the group consisting of biolyte 5-7 and biolyte 3-10, and (f) at least one ribonuclease selected from the group consisting of endonuclease and exonuclease, wherein an aqueous solution of (a) Tris; (b) urea and thiourea, (c) CHAPS, (d) DTT, (e) biolyte 3-10, and (f) endonuclease, is particularly preferred. The lysis buffer comprises (a) at least one buffer component, (b) at least one chaotrope, (c) at least one detergens, (d) at least one reducing agent (e) at least one carrier ampholyte, (f) at least one ribonuclease is particularly preferred.

In yet a further embodiment of the method, the protein of interest is a biomarker selected from the first group of said biomarkers or is a biomarker selected from the second group of said biomarkers, in particular is selected from the first group consisting of A1AT1, A1AT6, A2MG, PROP, TTHY, A1AG8, APOA1, APOC3, APOH, GPX3, RETBP, SAMP, or more preferably, is selected from the first group consisting of A1AG8, APOA1, APOC3, APOH, GPX3, RETBP, SAMP, or is selected from the second group consisting of APOE, or the protein of interest is a biomarker selected from the group of ApoA4, ApoM, a-raf, fetuin B, GSN, PLG, VPS28, wherein fetuin B and/or PLG are particularly preferred.

Within the context of peptide mass fingerprinting, it is particularly preferred if the amount of A1AT1, A1AT6, A2MG, PROP, TTHY, A1AG8, APOA1, APOC3, APOH, GPX3, RETBP, SAMP and/or APOE is determined by determining the amount of the peptide fragments thereof, as described herein, in the digest mixture.

In another embodiment of the method the digesting buffer comprises a bicarbonate compound and a protease, wherein the digesting buffer preferably is an aqueous solution of at least one bicarbonate compound selected from the group consisting of ammonium bicarbonate and sodium bicarbonate and of at least one serine protease, in particular selected from the group consisting of trypsin, chymotrypsin and elastase, or, in particular preferred, the digesting buffer is an aqueous solution of ammonium bicarbonate and trypsin.

In yet another embodiment of the method, the mass spectrometry is selected from the group consisting of MALDI-TOF and ESI-TOF, preferably the mass spectrometry is performed by MALDI-TOF.

In a further embodiment of the method, a tandem mass spectrometer is used for the peptide mass fingerprinting, wherein a MALDI-TOF/TOF spectrometry is particularly preferred for putting the method into practice.

In yet a further embodiment of the method, a matrix is used for the mass spectrometry selected from the group consisting of 3,5-dimethoxy-4-hydroxycinnamic acid, a-cyano-4-hydroxycinnamic acid and 2,5-dihydroxybenzoic acid, wherein a-cyano-4-hydroxycinnamic acid is particularly preferred as the matrix.

In another preferred embodiment of the method, the serum sample is calibrated or the serum samples are equilibrated to a predefined protein concentration by adding the lysis buffer, thus allowing an easy adaption of the system to different purposes.

In particular, it is preferred, if the method further comprises the steps of

determining the protein concentration of the serum sample, in particular by the Bradford method; or freezing and thawing the serum sample before the lysis buffer is added; and/or staining the gel after the 2-DE, in particular by using coomassie blue; and/or destaining the exised sample; or shrinking, in particular by adding acetonitrile, and drying of the excised sample before the digesting buffer is added; or using a peptide calibration standard for the mass spectrometry, wherein preferably a combination of said steps, in particular two of said steps, more preferably three of said steps, in particular four or five of said steps, most preferably all of said steps are implemented.

Yet another aspect of the invention concerns a procedure to screen for and to identify drugs against cancer associated with an increased c-myc activity, wherein the procedure comprises determining in a body fluid sample of a transgenic cancer mouse being treated with a compound to be tested, in particular of a mouse whose genome comprises a non natural c-myc sequence, at least one biomarker selected from the first group of said biomarkers and/or at least one biomarker selected from the second group of said biomarkers, and wherein the body fluid level of the at least one biomarker of said first group being significantly lower and/or the body fluid level of the at least one biomarker of said second group being significantly higher than the level of said biomarker(s) in the body fluid of an untreated transgenic cancer mouse is indicative of the therapeutic effect of said compound as an c-myc activity modulator.

In yet a further aspect, the invention concerns a procedure to screen for and to identify drugs against cancer or dysplasia, respectively, associated with an aberrant EGF receptor tyrosine kinase signalling, in particular against lung cancer or bronchial dysplasia, comprising determining in a body fluid sample of a transgenic cancer mouse being treated with a compound to be tested, in particular of a mouse whose genome comprises a non natural EGF sequence, at least one biomarker selected from the group of fetuin B, GSN, VPS28 or their fragments according to one of the claims 8 b), 8 d), 9 and/or at least one biomarker selected from the group of ApoA4, ApoM, a-raf, PLG or their fragments according to one of the claims 8 b), 8 d), 9 wherein the body fluid level of the at least one biomarker of said first group being significantly lower and/or the body fluid level of the at least one biomarker of said second group being significantly higher than the level of said biomarker(s) in the body fluid of an untreated transgenic cancer mouse is indicative of the therapeutic effect of said compound as a EGF receptor tyrosine kinase activity modulator for treating said cancer or as a chemopreventive drug for treating said dysplasia, respectively.

According to another aspect of the invention a procedure for identifying diagnostic cancer biomarkers is provided, comprising the steps of

isolating serum samples from blood samples of a plurality of c-myc cancer mice bearing the same type of tumor;

adding lysis buffer to said serum samples;

separating the proteins of said lysed serum samples by 2-DE gel electrophoreses;

excising from the gels each one sample containing a protein of interest,

adding digesting buffer to the excised samples;

analyzing the digest mixtures by mass spectrometry, in particular by peptide mass fingerprinting, and determining the protein of interest as biomarker, wherein a serum level of the protein being significantly higher and/or a serum level of the protein being significantly lower than the level of said protein in the serum of normal subjects is indicating the biomarker.

Still another aspect of the invention relates to a procedure for identifying diagnostic dysplasia biomarkers, comprising the steps of

isolating serum samples from blood samples of a plurality of EGF cancer mice having the same type of abnormality in maturation of cells, in particular having low grade or high grade bronchial dysplasia;

adding lysis buffer to said serum samples;

separating the proteins of said lysed serum samples by 2-DE gel electrophoreses;

excising from the gels each one sample containing a protein of interest,

adding digesting buffer to the excised samples;

analyzing the digest mixtures by mass spectrometry, in particular by peptide mass fingerprinting, and determining the protein of interest as biomarker, wherein a serum level of the protein being significantly higher and/or a serum level of the protein being significantly lower than the level of said protein in the serum of normal subjects is indicating the biomarker.

In a preferred embodiment, the procedure is implemented by using the method according the invention, in particular by using the method comprising an immunoassay or a peptide mass fingerprinting as described herein.

In particular it is preferred, and surprisingly sufficient, to implement the uses, methods and procedures according to the invention by performing a western blot, thus further simplifying the accomplishement of the invention in its different embodiments.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### EXAMPLES

Overview

Samples of transgenic lung tumor bearing mice and samples of healthy mice are used.

The protein concentration of samples which are examined is determined by means of

Bradford protein assay. The serum proteins of transgenic and healthy control animals are extracted in a lysis buffer containing thiourea and isolated by 2-D gel electrophoresis according to their individual iso-electrical point as well as to their molecular weight.

After colloidal Coomassie blue staining of the 2-D gels, the coloured gel spots are quantified by means of software and significant differences between healthy and ill mice are determined. Subsequently, the differing spots are excised, destained and digested with trypsin. The peptides received are characterized by matrix-assisted laser desorption/ionization mass spectrometry (MALDI-TOF/TOF).

Histopathology

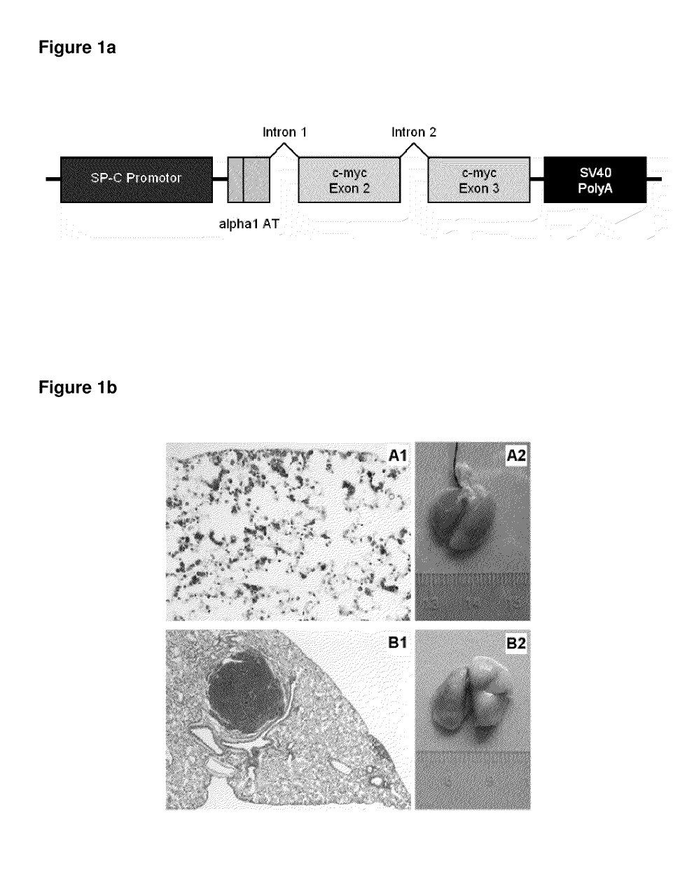

Transgenic lung tumor bearing mice are bred and kept as described previously. FIG. 1a depicts the gene construct while FIG. 1b (B1, B2) displays typical features of the lung tumors. Tissues are fixed in 4% buffered paraformaldehyde for approximately 20 h, dehydrated and embedded in paraffin (Roti-Plast™, Roth). Tissue captions are stained with hematoxylin and eosin according to standard protocols. The mouse tumors are classified according to the IARC—WHO (2004).

Sample Preparation

Blood serum of lung tumor bearing SP-C/c-myc mice (n=6, aged 14 months) and blood serum of healthy non-transgenic mice (n=6, aged 14 months) that served as controls, are studied. Blood from healthy and tumor bearing mice is withdrawn from the vena cava. After clotting for 2 h at room temperature, the blood is centrifuged at 3500 rpm for 15 min. The resultant supernatants are removed and frozen immediately in liquid nitrogen and stored at −80° C. until further analysis. The protein concentration in serum is determined by the Bradford protein assay (BioRad).

Two-Dimensional Gel-Electrophoresis

Each sample is analyzed in triplicate. Serum proteins are separated by isoelectric focusing (IEF) with precast IPG strips (pH 3-10, non-linear gradient and pH 4-7, linear gradient; both 170×3×0.5 mm, BioRad). 800 μg is diluted in a lysis buffer containing 2 mol/L thiourea, 5 mol/L urea, 40 mmol/L Tris, 4% CHAPS, 0.5% BioLyte 3-10 (BioRad), 100 mmol/L DTT resulting in a total volume of 350 μL per strip. Focused IPG strips are rehydrated at 50 V for 12 h. IEF is performed at 20° C. with a maximum voltage of 10 kV and a maximum current of 50 μA per strip.

After IEF, IPG strips are equilibrated in 10 mL reducing buffer (2% DTT in 10 mL equilibration buffer containing 6 mol/L urea, 30% glycerin, 2% SDS, 0.05 mol/L Tris-HCl, pH 8.8 and 0.5% bromphenol blue) for 15 min, followed by equilibration in 10 mL alkylation buffer (4% iodoacetamide and 0.5% bromphenol blue in 10 mL equilibration buffer) for 15 min.

SDS-PAGE is performed in a Protean-plus Dodeca ^{\II} Cell (BioRad) using self-cast polyacrylamide gels (200×205×1.5 mm; 12% T). Gels are run in parallel in 0.025 mol/L Tris/0.192 mol/L glycine/ 0.1% SDS at 10° C. with a constant voltage of 70 V overnight. Precision Plus Protein Unstained Standard™ (BioRad) is used for calibration of Mᵣ and pI.

Staining and Imaging

2-D Gels are fixed overnight in 500 mL 30% ethanol/ 2% phosphoric acid and washed three times for 20 min each in 500 mL 2% phosphoric acid. Equilibration is done with 500 mL 2% phosphoric acid/ 18% ethanol/ 15% ammonium sulfate thereafter. Colloidal Coomassie Brilliant Blue (CBB) staining of proteins is started by addition of 5 mL staining solution (2% CBB G250, Roth) to 500 mL of equilibration solution. Staining is carried out for 48 h and thereafter washed once with 500 mL water for 10 min.

Gels are scanned with the Perfection 4990 Photo™ densitometer (Epson). Detection of spots, quantification and comparison of 2-D protein profiles is done with the PDQuest 8.0 software (BioRad). After removal of background and vertical streaks from each gel image spots are digitized by Gaussian fit. To quantify protein spots, a matchset of all gels is made and the absorbance of individual protein spots from 2-D gels is measured. The raw quantity of each spot in a member gel is divided by the total intensity value of all the pixels in the image, for instance total density in gel image. This normalization procedure of the software assumed that the total density of an image, consisting of background and spot density will be relatively consistent from gel to gel.

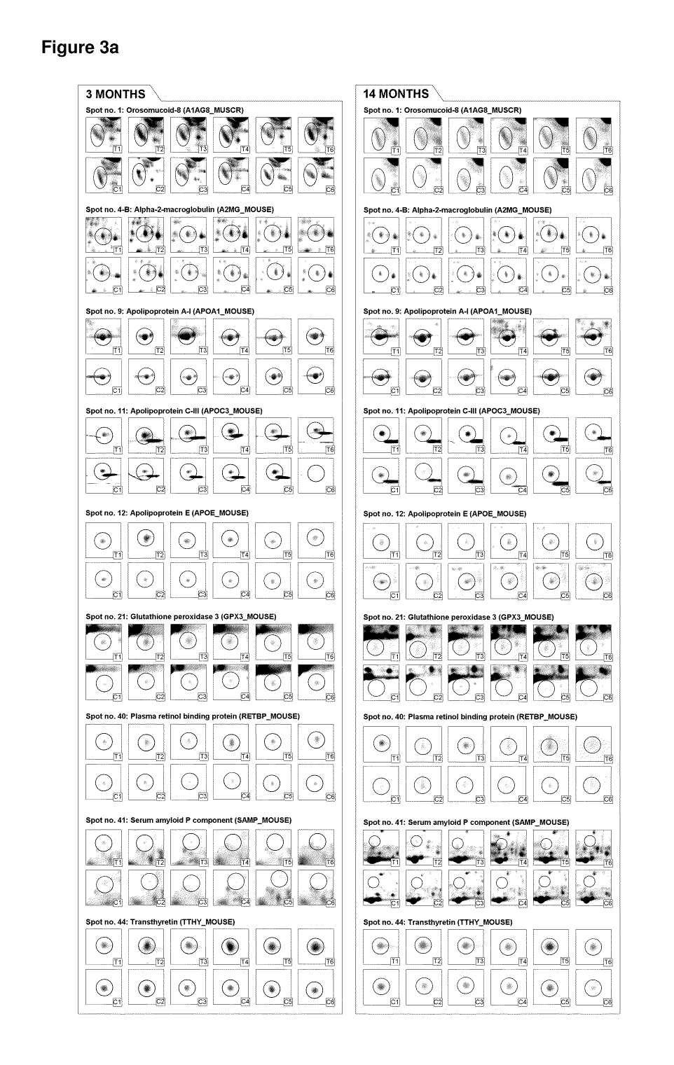

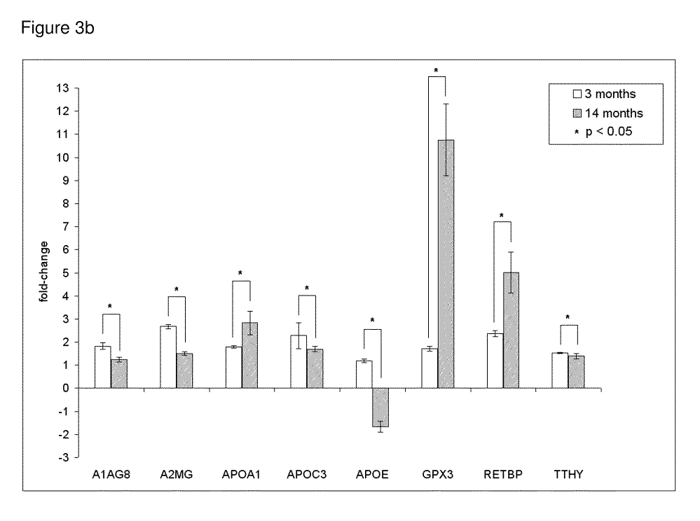

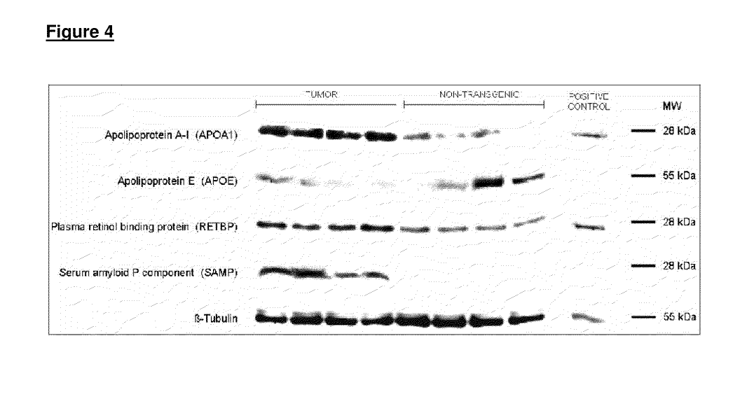

The expression of serum proteins is analyzed by the Student's t-test. A probability of p<0.05 is considered statistically significant (Tab. 2). Graphical evaluation is performed with the SigmaPlot (SPSS) software (FIG. 3b). Spots are excised and transferred to 96-well microtiter plates (ABgene) by the EXQuest™ spot cutter (BioRad) as described previously.

Mass Spectrometry

In-Gel Digestion

Each of the CBB-stained gel plugs is washed twice with 15 μL ammonium hydrogencarbonate solution (100 mmol/L) and then dehydrated twice with 15 μL acetonitrile. Proteins are digested with a total of 160 μg trypsin (13 ng/μL, sequencing grade, Promega) per gel plug at 37° C. for 4 h. Resulting peptides are extracted with 8 μL n-Octyl-B-D-glucopyranoside (5 mmol/L, Applichem)/ 1% trifluoroacetic acid in an ultrasonic bath (Sonorex, Super RK 514 BH, Bandelin) for 5 min.

MALDI-TOF/TOF

The HCCA matrix is prepared with the thin layer method. 1 μL of the peptide extracts were manually spotted onto a 600 μm/384 well AnchorChip™ sample target (Bruker Daltonics) and dried at ambient temperature. Recristallization was performed with 1 μL of 60% ethanol/30% acetone/ 10% of 1% trifluoroacetic acid thereafter. MALDI mass spectra are recorded using a Ultraflex II TOF/TOF mass spectrometer (Bruker Daltonics) equipped with a 384-sample scout source. A peptide calibration standard (Bruker Daltonics) is used for external calibration. MS and MS/MS data are recorded automatically on the MALDI-TOF/TOF instrument using the three most abundant peptide signals of the corresponding peptide mass fingerprint (PMF) spectrum. The Swiss-Prot database employing the Mascot program (version 2.0, Matrix Science, in-house server) is used for the search of peptide masses to identify proteins. Database searches are performed taking into account carbamidomethyl modification of cysteines and possible oxidation of methionine. One missed cleavage was allowed. A mass accuracy of ≦100 ppm is requested for PMF. For MS/MS searches, a mass accuracy of ≦70 ppm is requested for peptide masses and their fragments, respectively. Identified proteins are sent to the Proteinscape™ database (Protagen) and checked individually for further consideration.

Results and Discussion

Separation of Serum Proteins by 2-DE

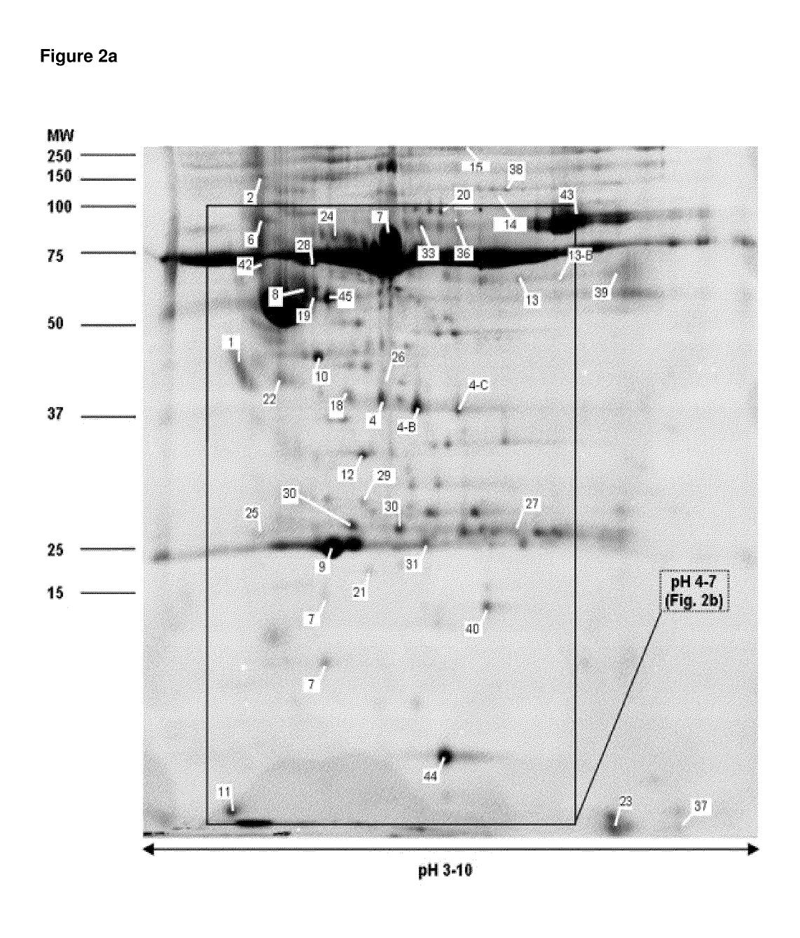

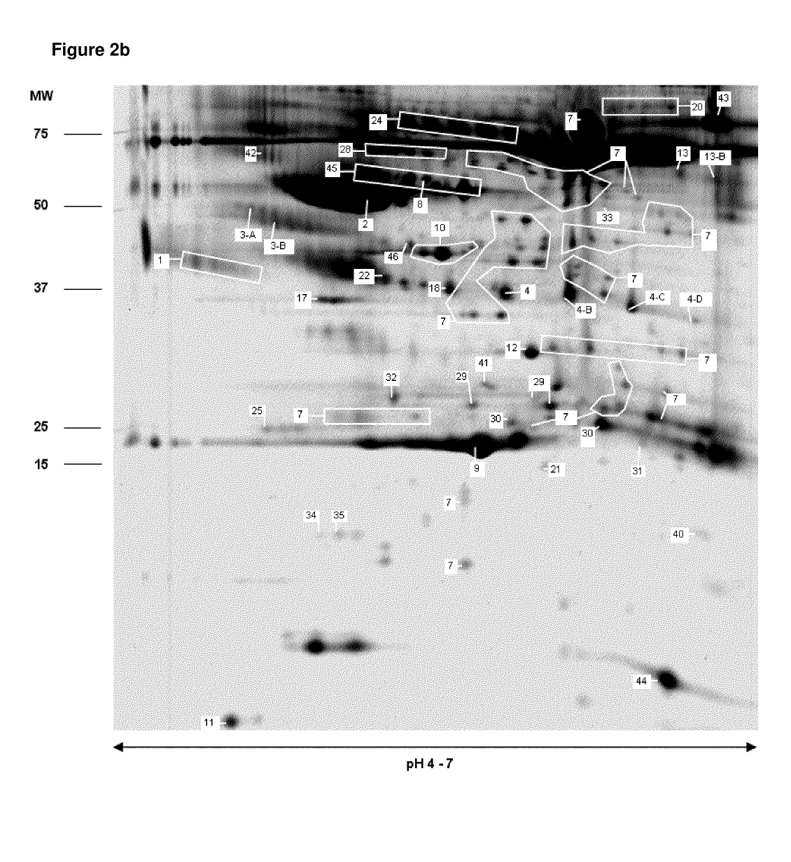

A thiourea-containing lysis buffer is used to extract proteins from serum. Proteins are separated within pH ranges of 3-10 and 4-7. Proteins are visualized with the colloidal CBB (CCB) stain. Approximately 400 (pH 3-10) and 200 (pH 4-7) spots per gel are detected. FIG. 2a and FIG. 2b depict examples of serum proteome maps (pH 3-10 and 4-7) of c-myc tumor bearing mice.

Protein Identification

72 2-D gels are stained with the CCB method. About 350 (pH 3-10) and 170 (pH 4-7) spots per gel are excised, resulting in 12.500 spots in total. Protein spots are analyzed by MALDI-MS and -MS/MS after tryptic in-gel digest. Identification is based on Swiss-Prot database entries with the Mascot search engine. In tumor bearing and healthy non-transgenic mice, 46 common (Tab. 1) serum proteins are identified. In Supplementary Table 1 the Mascot score, sequence coverage and the most informative peptide sequences identified by MS is listed. Within 2-DE mouse serum proteome mapping, three novel serum proteins, not reported so far, are identified (Tab. 1). These include orosomucoid-8 (spot no. 1), C-reactive protein (spot no. 18) and cytokeratin-8 (spot no. 26).

Regulation of Serum Proteins in Lung Cancer

Tab. 2 and FIG. 3b show expression profiles of 13 proteins (p <0.05) when extracts of healthy and lung tumor serum proteomes are compared, whereas FIG. 3a depicts prominent examples of regulated proteins in tumor bearing mice. Differentially expressed proteins are discussed below for their disease association. Their relationship to the c-myc proto-oncogene is determined by comparison of database entries from the MYC Cancer Gene website maintained by Johns Hopkins University. Furthermore, results are compared with another study on the serum proteome of c-raf transgenic mice which developed lung tumors as well.

Acute Phase Proteins

As observed in serum of healthy and tumor bearing mice of our present study, eight acute phase proteins are regulated. The positive acute phase proteins, e.g. alpha-1 acid glycoprotein-8, also named orosomucoid-8 (spot no. 1), alpha-1 antitrypsin (spot no. 2 and spot no. 3), alpha-2 macroglobulin (spot no. 4) and serum amyloid P component (spot no. 41) are found to be increased or exclusively expressed in tumor bearing mice. In contrast, the negative acute phase proteins (n-APP) plasma retinol-binding protein (spot no. 40), transthyretin (spot no. 44) and serum albumin (spot no. 7) are either up- or downregulated.

In particular, alpha-2 macroglobulin (A2M, spot no. 4) is mainly produced in the liver, but in the lung as well. With a molecular weight of 165 kDa, A2M represents a large plasma protein that consists of four identical subunits that are linked together by disulfide bonds. These subunits are visible at 37 kDa in our 2-D gel (FIG. 2b). Specifically, A2M acts as a proteinase inhibitor and targets serine-, cysteine-, aspartic- and metalloproteinases. The A2M structure includes a “decoy” region where such proteinases are bound and cleaved. Macrophage receptors recognize and eliminate the A2M-proteinase complex.

Serum A2M levels are useful for diagnosis and therapeutic monitoring in lung cancer and in bone metastases of prostate cancer as reported elsewhere.

Here a significant (p<0.05), >6-fold (pH 4-7) and 1.5-fold (pH 3-10) increase of alpha-2 macroglobulin levels in serum of tumor bearing mice is demonstrated.

Serum amyloid P component (SAMP, spot no. 41) is a member of the pentraxins, produced in the liver. SAMP has a sequence homology of 51% with the C-reactive protein (spot no. 18), a classical plasma APP as well Likewise, SAMP is exclusively expressed in tumor bearing mice.

Transthyretin, also named prealbumin (spot no. 44) is a common blood protein. It is a carrier for thyroid hormones from bloodstream to tissues. Transthyretin interacts with the retinol binding protein (RBP, spot no. 40), thus enabling retinol transportation. If transthyretin is not expressed, lower levels of retinol and RBP are observed

Since a decrease of transthyretin levels in serum is linked to a negative acute phase reaction during inflammation as well, an increase of this protein might be a significant biomarker for cancers.

Likewise, upregulation of transthyretin by 1.4-fold in serum of lung tumor bearing mice alongwith expression of RBP by 2.5-fold (pH 4-7) and 5-fold (pH 3-10) is demonstrated.

Apolipoproteins

Several apolipoproteins regulated in tumor bearing mice are found. Here the differential expression of apolipoproteins A, C, E and H in c-myc transgenic mice is reported.

Apolipoprotein A-I (ApoA1, spot no. 9) belongs to the ApoA1/A4/E protein family and is primarily produced in the liver and the intestine. ApoA1 is found in the extracellular space and, being a structural component of high density lipid proteins (HDL), takes part in cholesterol absorption.

The ApoA1 expression is found to be significantly increased by 1.4-fold (pH 4-7) and 2.8-fold (pH 3-10) in tumor bearing mice as well.

Spot no. 11 is identified as apolipoprotein C3 (ApoC3) which is produced in the liver, inhibits the lipoprotein and hepatic lipase and represses the uptake of lymph chylomicrons by hepatic cells. Thus, ApoC3 may repress the catabolism of triglyceride-rich particles. Upregulation of ApoC3 is demonstrated in a chronic renal failure model and in diabetes. A regulation of ApoC3 in lung cancer is not reported so far. Serum levels of ApoC3 are increased by 2.7-fold (pH 4-7) and 1.7-fold (pH 3-10) in lung tumor bearing mice.

Apolipoprotein E (ApoE, spot no. 12) is a mediator for binding, internalizing and metabolism of lipoprotein particles. It serves as a ligand for the low density lipoprotein (LDL) receptor and for the ApoE receptor (chylomicron remnant) of hepatic tissues. ApoE expression is reduced in serum of tumor bearing mice by 1.6-fold (pH 4-7) and 1.2-fold (pH 3-10).

Apolipoprotein H, also named as beta-2 glycoprotein-1 (ApoH, spot no. 13) binds to various kinds of negatively charged substances such as heparin, phospholipids and dextran sulfate. Through binding to phospholipids on the surface of damaged cells, ApoH inhibits activation of the intrinsic blood coagulation cascade. ApoH is synthesized in the liver and secreted into plasma.

ApoH expression is induced in sera of lung tumor bearing mice by 1.4-fold (pH 4-7) and 1.7-fold (pH 3-10) and is a novel finding for its regulation in lung cancer.

Oxidative Defense and Complement Activation

Glutathione peroxidase 3 (Gpx3, spot no. 21) functions in response to oxidative damage by catalyzing the reduction of hydrogen peroxide, lipid peroxides and organic hydroperoxide

An upregulation of Gpx3 in serum of lung tumor bearing mice by >10-fold (pH 3-10) is observed.

Properdin (spot no. 39), also known as factor P, is a serum glycoprotein and positive regulator of the alternate pathway for complement activation. It binds to and stabilizes the C3- and C5-convertase enzyme complexes. Complement C3 (spot no. 17) is not regulated in tumor bearing mice whereas properdin expression is increased by 1.5-fold. Properdin plays a role in some specific immune responses and in tissue inflammation.

Table Captions

Table 1: Protein identification in 2-DE maps of mouse serum proteins including

healthy and lung tumor bearing mice, identified by MALDI MS. A total of 46 proteins are identified. See Supplementary Table 1 for detailed information.

Table 2: Expression profiles of 13 significantly regulated proteins (p <0.05) from 2-D gels with pH 4-7 and pH 3-10. Quantification of protein abundance is done using the PDQuest 2-D software (BioRad) by measurement of the normalized optical density (arbitrary units, AU) of each protein spot. The change in abundance of the proteins is expressed by the calculated ratio (T/C) between mean values from tumor (T) and healthy (C) samples. % RSD: percental relative standard deviation. Spot no. 41 (SAMP_MOUSE) is exclusively expressed in tumor bearing mice. Student's t-test is used for calculation of p-values.

Table 3: Common and specific regulated proteins in adenomatous hyperplasia (AAH) and bronchiolo-alveolar adenocarcinomas (BAC). Recently, AAH is studied in SP-C/c-raf transgenic mice, whereas BAC is the subject of our present work on SP-C/c-myc transgenic mice. Yes/No: protein regulation, Yes*: exclusive protein expression either in healthy or tumor bearing mice.

Supplementary Table 1:

A summary of mouse serum proteins. The Mascot score (PMF), ions score (PFF), the number of identified peptides, their sequence, the protein coverage of the best hits and supporting information are shown for each identified protein. O@M: oxidation at the amino acid methionine.

Supplementary Table 2:

Investigating the mouse serum proteome. 46 distinct proteins in healthy and tumor bearing mice are identified. From these, 3 proteins are novel and not reported so far in 2-D mouse serum proteome maps, for instance, by Duan et al. (38 distinct proteins), Wait et al. (30 distinct proteins) our recent serum proteomics study on c-raf transgenic mice (45 distinct proteins) and Hood et al.

TABLE 1

| | | | | c-myc | |
|---|---|---|---|---|---|
| No. | Protein ID | Accession | Protein name | target | APP |
| 1 | A1AG8_MUSCR | P21352 | Orosomucoid-8 | No | Yes |
| 2 | A1AT1_MOUSE | P07758 | Alpha-1-antitrypsin 1-1 | No | Yes |
| 3 | A1AT6_MOUSE | P81105 | Alpha-1-antitrypsin 1-6 | No | Yes |
| 4 | A2MG_MOUSE | Q61838 | Alpha-2-macroglobulin | Yes | Yes |
| 5 | ACTG_MOUSE | P63260 | Gamma-actin | Yes | No |
| 6 | AFAM_MOUSE | O89020 | Afamin | No | No |
| 7 | ALBU_MOUSE | P07724 | Serum Albumin | Yes | Yes |
| 8 | ANT3_MOUSE | P32261 | Antithrombin-III | No | No |
| 9 | APOA1_MOUSE | Q00623 | Apolipoprotein A-I | No | No |
| 10 | APOA4_MOUSE | P06728 | Apolipoprotein A-IV | No | No |
| 11 | APOC3_MOUSE | P33622 | Apolipoprotein C-III | No | No |
| 12 | APOE_MOUSE | P08226 | Apolipoprotein E | No | No |
| 13 | APOH_MOUSE | Q01339 | Apolipoprotein H | No | No |
| 14 | CFAB_MOUSE | P04186 | Complement factor B | Yes | No |
| 15 | CFAH_MOUSE | P06909 | Complement factor H | No | No |
| 16 | CLUS_MOUSE | Q06890 | Clusterin (Apolipoprotein J) | No | No |
| 17 | CO3_MOUSE | P01027 | Complement C3 | No | Yes |
| 18 | CRP_MOUSE | P14847 | C-reactive protein | No | Yes |
| 19 | FETUA_MOUSE | P29699 | Fetuin-A | Yes | No |
| 20 | GELS_MOUSE | P13020 | Gelsolin | No | No |
| 21 | GPX3_MOUSE | P46412 | Glutathione peroxidase 3 | No | No |
| 22 | HA10_MOUSE | P01898 | H-2 class I histocompatibility antigen, | No | No |
| | | | Q10 alpha chain | | |
| 23 | HBA_MOUSE | P01942 | Hemoglobin subunit alpha | Yes | No |
| 24 | HEMO_MOUSE | Q91X72 | Hemopexin | No | No |
| 25 | IGJ_MOUSE | P01592 | Immunglobulin J chain | No | No |
| 26 | K2C8_MOUSE | P11679 | Cytokeratin-8 | No | No |
| 27 | KAC_MOUSE | P01837 | Immunglobulin kappa chain C region | No | No |
| 28 | KNG1_MOUSE | O08677 | Kininogen-1 | No | No |
| 29 | KV3B_MOUSE | P01655 | Immunglobulin kappa chain V-III | No | No |
| | | | region PC 7132 | | |
| 30 | KV5L_MOUSE | P01645 | Immunglobulin kappa chain V-V | No | No |
| | | | region HP 93G7 | | |
| 31 | KV6K_MOUSE | P04945 | Immunglobulin kappa chain V-VI | No | No |
| | | | region NQ2-6.1 | | |
| 32 | MBL2_MOUSE | P41317 | Mannose-binding protein C | No | No |
| 33 | MUC_MOUSE | P01872 | Immunglobulin mu chain C region | No | No |
| | | | secreted form | | |
| 34 | MUP2_MOUSE | P11589 | Major urinary protein 2 | No | No |
| 35 | MUP8_MOUSE | P04938 | Major urinary proteins 11 and 8 | No | No |
| 36 | MYH9_MOUSE | Q8VDD5 | Myosin-9 | No | No |
| 37 | PLF4_MOUSE | Q9Z126 | Platelet factor 4 | No | No |
| 38 | PLMN_MOUSE | P20918 | Plasminogen | No | No |
| 39 | PROP_MOUSE | P11680 | Properdin | No | No |
| 40 | RETBP_MOUSE | Q00724 | Plasma retinol-binding protein | No | Yes |
| 41 | SAMP_MOUSE | P12246 | Serum amyloid P-component | No | Yes |
| 42 | SPA3K_MOUSE | P07759 | Contrapsin | No | No |
| 43 | TRFE_MOUSE | Q921I1 | Serotransferrin | No | Yes |
| 44 | TTHY_MOUSE | P07309 | Transthyretin | No | Yes |
| 45 | VTDB_MOUSE | P21614 | Vitamin D-binding protein | No | No |
| 46 | ZA2G_MOUSE | Q64726 | Zinc-alpha-2-glycoprotein | No | No |

TABLE 2

| | | | Tumor | | | | | | | |
|---|---|---|---|---|---|---|---|---|---|---|
| No. | pH | Protein ID | T1 | T2 | T3 | T4 | T5 | T6 | mean | % RSD |
| 1 | pH 4-7 | A1AG8_MUSCR | 1139 | 1003 | 1123 | 998 | 1217 | 867 | 1058 | 11.88 |
| 1 | pH 3-10 | A1AG8_MUSCR | 834 | 745 | 769 | 895 | 744 | 867 | 809 | 8.07 |
| 2 | pH 4-7 | A1AT1_MOUSE | 474 | 456 | 494 | 485 | 541 | 391 | 473 | 10.39 |
| 2 | pH 3-10 | A1AT1_MOUSE | 518 | 616 | 549 | 863 | 636 | 663 | 641 | 18.99 |
| 3 | pH 4-7 | A1AT6_MOUSE | 781 | 848 | 891 | 899 | 884 | 916 | 870 | 5.61 |
| 3 | pH 4-7 | A1AT6_MOUSE | 450 | 546 | 502 | 446 | 680 | 486 | 518 | 16.82 |
| 3 | pH 3-10 | A1AT6_MOUSE | 0 | 0 | 0 | 0 | 0 | 0 | 0 | 0 |
| 4 | pH 4-7 | A2MG_MOUSE | 5588 | 5789 | 4708 | 5609 | 5332 | 6008 | 5506 | 8.19 |
| 4 | pH 3-10 | A2MG_MOUSE | 6773 | 6059 | 6128 | 6592 | 5845 | 6307 | 6284 | 5.52 |
| 9 | pH 4-7 | APOA1_MOUSE | 5423 | 5219 | 4868 | 5172 | 4972 | 5267 | 5154 | 3.93 |
| 9 | pH3-10 | APOA1_MOUSE | 5952 | 4324 | 6205 | 5549 | 5869 | 7721 | 5937 | 18.47 |
| 11 | pH 4-7 | APOC3_MOUSE | 3433 | 3693 | 3472 | 3132 | 3217 | 3552 | 3417 | 6.02 |
| 11 | pH 3-10 | APOC3_MOUSE | 3682 | 3357 | 3318 | 3716 | 3217 | 3885 | 3529 | 7.57 |
| 12 | pH 4-7 | APOE_MOUSE | 2502 | 2139 | 2619 | 2204 | 2553 | 2267 | 2381 | 8.48 |
| 12 | pH 3-10 | APOE_MOUSE | 1857 | 1727 | 1294 | 1657 | 1553 | 1834 | 1654 | 12.65 |
| 13 | pH 4-7 | APOH_MOUSE | 2377 | 2773 | 2292 | 2783 | 2365 | 2131 | 2454 | 10.85 |
| 13 | pH 3-10 | APOH_MOUSE | 1148 | 1281 | 1269 | 1724 | 1247 | 1410 | 1347 | 15.08 |
| 21 | pH 4-7 | GPX3_MOUSE | 520 | 339 | 547 | 506 | 358 | 541 | 469 | 20.13 |
| 21 | pH 3-10 | GPX3_MOUSE | 189 | 223 | 240 | 233 | 213 | 290 | 231 | 14.62 |
| 39 | pH 4-7 | PROP_MOUSE | 0 | 0 | 0 | 0 | 0 | 0 | 0 | 0 |
| 39 | pH 3-10 | PROP_MOUSE | 284 | 311 | 304 | 248 | 240 | 240 | 271 | 12.02 |
| 40 | pH 4-7 | RETBP_MOUSE | 1244 | 1185 | 1534 | 1450 | 1225 | 1442 | 1347 | 10.83 |
| 40 | pH 3-10 | RETBP_MOUSE | 407 | 320 | 537 | 402 | 367 | 427 | 410 | 17.74 |
| 41 | pH 4-7 | SAMP_MOUSE | 638 | 1271 | 1350 | 724 | 523 | 1496 | 1000 | 41.86 |
| 41 | pH 3-10 | SAMP_MOUSE | 411 | 967 | 750 | 901 | 501 | 800 | 722 | 30.64 |
| 44 | pH 4-7 | TTHY_MOUSE | 9952 | 9049 | 9204 | 8992 | 9869 | 10721 | 9631 | 7.01 |
| 44 | pH 3-10 | TTHY_MOUSE | 9569 | 10047 | 8934 | 8170 | 8330 | 9154 | 9034 | 7.95 |

| | Control | | | | | | | | Ratio | |
|---|---|---|---|---|---|---|---|---|---|---|
| No. | C1 | C2 | C3 | C4 | C5 | C6 | mean | % RSD | (T/C) | P |
| 1 | 425 | 398 | 401 | 414 | 502 | 387 | 421 | 9.91 | 2.5 | 3.50 × 10⁻⁰⁷ |
| 1 | 780 | 637 | 545 | 715 | 679 | 552 | 651 | 14.20 | 1.2 | 6.64 × 10⁻⁰³ |
| 2 | 400 | 384 | 482 | 314 | 391 | 327 | 383 | 15.65 | 1.2 | 1.73 × 10⁻⁰² |
| 2 | 498 | 344 | 475 | 555 | 475 | 562 | 485 | 16.26 | 1.3 | 2.49 × 10⁻⁰² |
| 3 | 630 | 686 | 752 | 618 | 812 | 711 | 702 | 10.51 | 1.2 | 8.90 × 10⁻⁰⁴ |
| 3 | 349 | 354 | 306 | 359 | 410 | 484 | 377 | 16.46 | 1.4 | 8.85 × 10⁻⁰³ |
| 3 | 0 | 0 | 0 | 0 | 0 | 0 | 0 | 0 | 0 | 0 |
| 4 | 867 | 935 | 766 | 790 | 846 | 910 | 852 | 7.74 | 6.5 | 2.39 × 10⁻¹⁰ |
| 4 | 4176 | 4044 | 4087 | 4153 | 4900 | 3793 | 4192 | 8.89 | 1.5 | 1.51 × 10⁻⁰⁶ |
| 9 | 3490 | 4036 | 3362 | 3379 | 4004 | 3313 | 3597 | 9.25 | 1.4 | 1.93 × 10⁻⁰⁶ |
| 9 | 1603 | 1725 | 2017 | 2471 | 2260 | 2495 | 2095 | 18.04 | 2.8 | 1.04 × 10⁻⁰⁵ |
| 11 | 1313 | 1342 | 1236 | 1511 | 1189 | 1096 | 1281 | 11.17 | 2.7 | 1.57 × 10⁻⁰⁹ |
| 11 | 2372 | 1630 | 1749 | 2522 | 2131 | 2018 | 2070 | 16.71 | 1.7 | 9.73 × 10⁻⁰⁶ |
| 12 | 2478 | 2312 | 2823 | 2908 | 3116 | 2935 | 2762 | 11.02 | 0.9 | 2.85 × 10⁻⁰² |
| 12 | 2993 | 2207 | 2688 | 2876 | 2598 | 2978 | 2723 | 10.95 | 0.6 | 2.95 × 10⁻⁰⁵ |
| 13 | 1525 | 1330 | 1774 | 2073 | 2020 | 1947 | 1778 | 16.66 | 1.4 | 1.97 × 10⁻⁰³ |
| 13 | 845 | 752 | 809 | 898 | 749 | 683 | 790 | 9.75 | 1.7 | 9.11 × 10⁻⁰⁵ |
| 21 | 165 | 124 | 179 | 232 | 229 | 147 | 179 | 24.35 | 2.6 | 4.66 × 10⁻⁰⁵ |
| 21 | 19 | 20 | 15 | 20 | 25 | 30 | 22 | 24.39 | 10.8 | 3.45 × 10⁻⁰⁸ |
| 39 | 0 | 0 | 0 | 0 | 0 | 0 | 0 | 0 | 0 | 0 |
| 39 | 228 | 156 | 182 | 139 | 217 | 187 | 185 | 18.42 | 1.5 | 1.16 × 10⁻⁰³ |
| 40 | 429 | 466 | 555 | 716 | 521 | 546 | 539 | 18.44 | 2.5 | 5.52 × 10⁻⁰⁷ |
| 40 | 66 | 106 | 87 | 63 | 94 | 74 | 82 | 20.67 | 5.0 | 8.00 × 10⁻⁰⁷ |
| 41 | 0 | 0 | 0 | 0 | 0 | 0 | 0 | 0.00 | T | 1.61 × 10⁻⁰⁴ |
| 41 | 0 | 0 | 0 | 0 | 0 | 0 | 0 | 0.00 | T | 1.19 × 10⁻⁰⁵ |
| 44 | 8045 | 7835 | 6190 | 9258 | 5998 | 5384 | 7118 | 20.91 | 1.4 | 3.68 × 10⁻⁰³ |
| 44 | 6148 | 6432 | 6932 | 6738 | 6676 | 6095 | 6504 | 5.18 | 1.4 | 1.44 × 10⁻⁰⁵ |

TABLE 3

| | Protein ID | Protein name | BAC | AAH |
|---|---|---|---|---|
| common | A1AT6_MOUSE | Alpha-1-antitrypsin 1-6 | Yes | Yes |
| | A2MG_MOUSE | Alpha-2-macroglobulin (A2M) | Yes | Yes |
| | TTHY_MOUSE | Transthyretin | Yes | Yes |
| | PROP_MOUSE | Properdin | Yes | Yes |
| specific | A1AG8_MOUSE | Orosomucoid-8 | Yes | No |
| | APOA1_MOUSE | Apolipoprotein A-I | Yes | No |
| | APOC3_MOUSE | Apolipoprotein C-III | Yes | No |
| | APOE_MOUSE | Apolipoprotein E | Yes | No |
| | APOH_MOUSE | Apolipoprotein H | Yes | No |
| | GPX3_MOUSE | Glutathione peroxidase 3 | Yes | No |
| | RETBD_MOUSE | Plasma retinol-binding protein | Yes | No |
| | SAMP_MOUSE | Serum amyloid P-component | Yes | No |
| | MUP8_MOUSE | Major urinary proteins (MUP) | No | Yes |
| | VTBD_MOUSE | Vitamin D-binding protein | No | Yes |
| | S6A11_MOUSE | Sodium- and chloride-dependent | No | Yes |
| | | GABA transporter 4 | | |
| | EGFR_MOUSE | Epidermal growth factor receptor | No | Yes |
| | | (EGFR) | | |

SUPPLEMENTARY TABLE 1
A summary of mouse serum proteins

| | | | | Mascot | | Ions score | | | |
|---|---|---|---|---|---|---|---|---|---|
| | | | | score(PMF) | | (PFF) | | | |
| | | | | No. of | | No. of | | | |
| | | | | matched | | matched | | | |
| | | Swissprot | | peptides | | peptides | Peptide | | |
| | Swissprot | Access | | Percentual | Peptide sequences | Percentual | sequences | | Subcellular |
| No. | ID | ion no. | Protein aliases | coverage | From PMF | coverage | from PFF | Gene | location |
| 1 | A1AG8_{—} | P21352 | Alpha-1-acid glycoprotein 8 | — | | 71 | YEGGVETFAHLIVLR | Orm8 | Secreted |
| | MUSCR | | [Precursor] | | | 1 | | Agp-8 | |
| | | | AGP 8 | | | 7 | | Orm-8 | |
| | | | Orosomucoid-8 | | | | | | |
| | | | OMD 8 | | | | | | |
| 2 | A1AT1_{—} | P07758 | Alpha-1-antitrypsin 1-1 | 100 | LAQIHFPR | | — | Serpin | Secreted |
| | MOUSE | | [Precursor] | 10 | TLMSPLGITR | | | a1a | |
| | | | Serine protease inhibitor 1-1 | 26 | TLMSPLGITR + O@M | | | Dom1 | |
| | | | Alpha-1 protease inhibitor 1 | | RLAQIHFPR | | | Spi1-1 | |
| | | | Alpha-1-antiproteinase | | ELISKFLLNR | | | | |
| | | | AAT | | LDQDTVFALANYILFK | | | | |
| | | | | | KLDQDTVFALANYILFK | | | | |
| | | | | | IFNNGADLSGITEENAPLK | | | | |
| | | | | | DQSPASHEIATNLGDFAISLYR | | | | |
| | | | | | KPFDPENTEEAEFHVDESTTVK | | | | |
| 3 | A1AT6_{—} | P81105 | Alpha-1-antitrypsin 1-6 | 120 | LAQIHFPR | 111, 42, 33 | LAQIHFPR | Spi1-6 | Secreted |
| | MOUSE | | [Precursor] | 11 | TLMSPLGITR | 3 | ELISKFLLNR | Dom6 | |
| | | | Serine protease inhibitor 1-6 | 34 | TLMSPLGITR + O@M | 9 | DQSPASHEIATNLGDF | | |
| | | | Alpha-1 protease inhibitor 6 | | RLAQIHFPR | | AISLYR | | |
| | | | | | MQHLEQTLSK | | | | |
| | | | | | ELISKFLLNR | | | | |
| | | | | | IFNNGADLSGITEENAPLK | | | | |
| | | | | | NHYQAEVFSVNFAESEEAK | | | | |
| | | | | | DQSPASHEIATNLGDFAISLYR | | | | |
| | | | | | KPFDPENTEEAEFHVDESTTVK | | | | |
| | | | | | FDHPFLFIIFEEHTQSPLFVGK | | | | |
| 4 | A2MG_{—} | Q61838 | Alpha-2-macroglobulin | — | — | 89 | APFALQVNTLPLNFDK | A2m | Secreted |
| | MOUSE | | [Precursor] | | | 1 | | Pzp | |
| | | | Alpha-2-M | | | 1 | | | |
| | | | Pregnancy zone protein | | | | | | |
| 5 | ACTG_{—} | P60710 | Actin, cytoplasmic | 101 | IIAPPERK | — | — | Actg1 | Cytoplasma |
| | MOUSE | | 2 Gamma-actin | 9 | AGFAGDDAPR | | | Actg | |
| | | | | 31 | GYSFTTTAER | | | | |
| | | | | | AVFPSIVGRPR | | | | |
| | | | | | IWHHTFYNELR | | | | |
| | | | | | QEYDESGPSIVHR | | | | |
| | | | | | SYELPDGQVITIGNER | | | | |
| | | | | | VAPEEHPVLLTEAPLNPK | | | | |
| | | | | | DLYANTVLSGGTTMYPGIADR + | | | | |
| | | | | | O@M | | | | |
| 6 | AFAM_{—} | O89020 | Afamin [Precursor] | 101 | FLVNLVK | — | — | Afm | Secreted |
| | MOUSE | | Alpha-albumin | 14 | TDFAFRR | | | | |
| | | | Alpha-Alb | 22 | ENPAGCYR | | | | |
| | | | | | AAPITQYLK | | | | |
| | | | | | VMLDYRDR + O@M | | | | |
| | | | | | HRFLVNLVK | | | | |
| | | | | | RLCFFYNK | | | | |
| | | | | | VLNSINVAVFSK | | | | |
| | | | | | FTESENVCQER | | | | |
| | | | | | VYMDFLEDCCSR + O@M | | | | |
| | | | | | AAPQLPMEELVSLSK + O@M | | | | |
| | | | | | ANVGFLPPFPTLDPEEK | | | | |
| | | | | | KANVGFLPPFPTLDPEEK | | | | |
| | | | | | EACIINANKDDRPEGLSLR | | | | |
| 7 | ALBU_{—} | P07724 | Serum albumin [Precursor] | 124 | CCTLPEDQR | 95 | LGEYGFQNAILVR | Alb | Secreted |
| | MOUSE | | | 11 | HPDYSVSLLLR | 1 | | Alb1 | |
| | | | | 24 | APQVSTPTLVEAAR | 2 | | Alb-1 | |
| | | | | | RHPDYSVSLLLR | | | | |
| | | | | | LGEYGFQNAILVR | | | | |
| | | | | | DVFLGTFLYEYSR | | | | |
| | | | | | LPCVEDYLSAILNR | | | | |
| | | | | | RPCFSALTVDETYVPK | | | | |
| | | | | | AETFTFHSDICTLPEK | | | | |
| | | | | | AADKDTCFSTEGPNLVTR | | | | |
| | | | | | CCAEANPPACYGTVLAEFQPLV | | | | |
| | | | | | EEPK | | | | |
| 8 | ANT3_{—} | P32261 | Antithrombin-III [Precursor] | 105 | RVWELSK | — | — | Serpin | Secreted |
| | MOUSE | | ATIII | 13 | SLNPNRVTFK | | | c1 | extra- |
| | | | | 33 | ANRPFLVLIR | | | At3 | cellular |
| | | | | | TSDQIHFFFAK | | | | space |
| | | | | | DIPVNPLCIYR | | | | |
| | | | | | EVALNTIIFMGR | | | | |
| | | | | | EVALNTIIFMGR + O@M | | | | |
| | | | | | SSDLVSANRLFGDK | | | | |
| | | | | | LQPLDFKENPEQSR | | | | |
| | | | | | DIPVNPLCIYRSPGK | | | | |
| | | | | | ATEEDGSEQKVPEATNR | | | | |
| | | | | | AFLEVNEEGSEAAASTSVVITG | | | | |
| | | | | | R | | | | |
| | | | | | SQLPGIVAGGRDDLYVSDAFHK | | | | |
| 9 | APOA1_{—} | Q00623 | Apolipoprotein A-I [Precursor] | 96 | LQELQGR | 84 | VAPLGAELQESAR | Apoa1 | Secreted |
| | MOUSE | | Apo-AI | 15 | EDVELYR | 1 | | | |
| | | | ApoA-I | 40 | ARPALEDLR | 4 | | | |
| | | | | | LSPVAEEFR | | | | |
| | | | | | QKLQELQGR | | | | |
| | | | | | WKEDVELYR | | | | |
| | | | | | VQPYLDEFQK | | | | |
| | | | | | TQLAPHSEQMR | | | | |
| | | | | | TQLAPHSEQMR + O@M | | | | |
| | | | | | LSPVAEEFRDR | | | | |
| | | | | | SNPTLNEYHTR | | | | |
| | | | | | VAPLGAELQESAR | | | | |
| | | | | | QEMNKDLEEVK | | | | |
| | | | | | VKDFANVYVDAVK | | | | |
| | | | | | LQELQGRLSPVAEEFR | | | | |
| 10 | APOA4_{—} | P06728 | Apolipoprotein A-IV [Precursor] | 235 | EAVEQFQK | — | — | Apoa4 | Secreted |
| | MOUSE | | Apo-AIV | 21 | LQLTPYIQR | | | | |
| | | | ApoA-IV | 64 | ALVQQLEQFR | | | | |
| | | | | | SLAPLTVGVQEK | | | | |
| | | | | | TVEPMGEMFNK + O@M | | | | |
| | | | | | QLEQQVEEFR | | | | |
| | | | | | NLAPLVEDVQSK | | | | |
| | | | | | TVEPMGEMFNK + 2 O@M | | | | |
| | | | | | NMEELKGHLTPR + O@M | | | | |
| | | | | | ATIDQNLEDLRR | | | | |
| | | | | | LGDASTYADGVHNK | | | | |
| | | | | | QLEQQVEEFRR | | | | |
| | | | | | LNHQMEGLAFQMK + 2 O@M | | | | |
| | | | | | TDVTQQLSTLFQDK | | | | |
| | | | | | LNHQMEGLAFQMKK + 2 O@M | | | | |
| | | | | | LVPFVVQLSGHLAKETER | | | | |
| | | | | | QQLGPNSGEVESHLSFLEK | | | | |
| | | | | | MMPHANKVTQTFGENMQK + 3 | | | | |
| | | | | | O@M | | | | |
| | | | | | LQEHLKPYAVDLQDQINTQTQE | | | | |
| | | | | | MK + O@M | | | | |
| | | | | | GSPDQPQALPLPEQAQEQAQE | | | | |
| | | | | | QAQEQVQPKPLES | | | | |
| | | | | | KGSPDQPQALPLPEQAQEQAQ | | | | |
| | | | | | EQAQEQVQPKPLES | | | | |
| 11 | APOC3_{—} | P33622 | Apolipoprotein C-III [Precursor] | — | — | 128 | TVQDALSSVQESDIAV | Apoc3 | Secreted |
| | MOUSE | | Apo-CIII | | | 1 | VAR | | |
| | | | ApoC-III | | | 19 | | | |
| 12 | APOE_{—} | P08226 | Apolipoprotein E [Precursor] | 108 | EVQAAQAR | 81 | ELEEQLGPVAEETR | Apoe | Secreted |
| | MOUSE | | Apo-E | 20 | FWDYLR | 1 | | | |
| | | | | 44 | DRLEEVR | 4 | | | |
| | | | | | EHMEEVR + O@M | | | | |
| | | | | | LGPLVEQGR | | | | |
| | | | | | LEEVGNQAR | | | | |
| | | | | | QWANLMEK | | | | |
| | | | | | DRAQAFGDR | | | | |
| | | | | | LQAEIFQAR | | | | |
| | | | | | MEEQTQQIR | | | | |
| | | | | | MEEQTQQIR + O@M | | | | |
| | | | | | GRLEEVGNQAR | | | | |
| | | | | | TANLGAGAAQPLR | | | | |
| | | | | | SKMEEQTQQIR | | | | |
| | | | | | SKMEEQTQQIR + O@M | | | | |
| | | | | | GWFEPIVEDMHR | | | | |
| | | | | | GWFEPIVEDMHR + O@M | | | | |
| | | | | | ELEEQLGPVAEETR | | | | |
| | | | | | NEVHTMLGQSTEEIR | | | | |
| | | | | | NEVHTMLGQSTEEIR + O@M | | | | |
| 13 | APOH_{—} | Q01339 | Beta-2-glycoprotein 1 [Precursor] | 153 | IHFYCK | — | — | Apoh | Secreted |
| | MOUSE | | Beta-2-glycoprotein I | 15 | ATVLYQGMR | | | B2gp1 | |
| | | | Apolipoprotein H | 49 | ATVLYQGMR + O@M | | | | |
| | | | Apo-H | | ITCPPPPVPK | | | | |
| | | | B2GPI | | WSPDIPACAR | | | | |
| | | | Beta(2)GPI | | DGTIEIPSCFK | | | | |
| | | | Activated protein C-binding | | CSYTVEAHCR | | | | |
| | | | protein | | TGTWSFLPTCR | | | | |
| | | | APC inhibitor | | VCPFAGILENGIVR | | | | |
| | | | | | IQEQFKNGMMHGDK | | | | |
| | | | | | FTCPLTGMWPINTLR | | | | |
| | | | | | FTCPLTGMWPINTLR + O@M | | | | |
| | | | | | ICPKPDDLPFATVVPLK | | | | |
| | | | | | TSYDPGEQIVYSCKPGYVSR | | | | |
| | | | | | CPFPPRPENGYVNYPAKPVLLY | | | | |
| | | | | | K | | | | |
| 14 | CFAB_{—} | P04186 | Complement factor B [Precursor] | 100 | CLTNLIEK | — | — | Cfb | Secreted |
| | MOUSE | | EC 3.4.21.47 | 12 | VASYGVRPR | | | Bf | |
| | | | C3/C5 convertase | 18 | QQLVPSYAR | | | H2-Bf | |
| | | | | | ALLDIGRDPK | | | | |
| | | | | | VKDASEVVTPR | | | | |
| | | | | | YGLLTYATVPK | | | | |
| | | | | | ALRLPQTATCK | | | | |
| | | | | | DLEIEEVLFHPK | | | | |
| | | | | | YGLLTYATVPKVLVR | | | | |
| | | | | | VGSQYRLEDIVTYHCSR | | | | |
| | | | | | YGQTLRPICLPCTEGTTR | | | | |
| | | | | | FIQVGVISWGVVDVCRDQR | | | | |
| 15 | CFAH_{—} | P06909 | Complement factor H [Precursor] | 116 | TGEQVTFR | — | — | Cfh | Secreted |
| | MOUSE | | Protein beta-1-H | 16 | ARDSPPFR | | | Hf1 | |
| | | | | 13 | HGYVPKER | | | | |
| | | | | | YLHGDRVR | | | | |
| | | | | | CVEILCTPPR | | | | |
| | | | | | IIHRSDDEIR | | | | |
| | | | | | CTPTGWIPVPR | | | | |
| | | | | | CLPVTELENGR | | | | |
| | | | | | SYRTGEQVTFR | | | | |
| | | | | | CVATDQLEKCR | | | | |
| | | | | | VLKSTGIEAIKPK | | | | |
| | | | | | VGDLLEFSCHSGHR | | | | |
| | | | | | TDCDVLPTVKNAIIR | | | | |
| | | | | | KKPCGHPGDTPFGSFR | | | | |
| | | | | | SCDMPVFENSITKNTR + O@M | | | | |
| | | | | | YRVGDLLEFSCHSGHR | | | | |
| 16 | CLUS_{—} | Q06890 | Clusterin [Precursor] | 68 | RPHFLYPK | — | — | Clu | Secreted |
| | MOUSE | | Sulfated glycoprotein 2 | 4 | ASGIIDTLFQDR | | | Apoj | |
| | | | SGP-2 | 12 | ELHDPHYFSPIGFPHK | | | Msgp-2 | |
| | | | Clustrin | | YYLRVSTVTTHSSDSEVPSR | | | | |
| | | | Apolipoprotein J | | | | | | |
| | | | Apo-J | | | | | | |
| 17 | CO3_MOUSE | P01027 | Complement C3 [Precursor] | 67 | WLNEQR | — | — | C3 | Secreted |
| | | | Synonym HSE-MSF | 10 | RQEALELIK | | | | |
| | | | | 8 | DFDSVPPVVR | | | | |
| | | | | | EADVSLTAFVLIALQEAR | | | | |
| | | | | | LLDDFDEYTMTIQQVIK + O@M | | | | |
| | | | | | IILQGSPVVQMAEDAVDGER | | | | |
| | | | | | VDVPAADLSDQVPDTDSETR | | | | |
| | | | | | IILQGSPVVQMAEDAVDGER + | | | | |
| | | | | | O@M | | | | |
| | | | | | VFSLAANLIAIDSHVLCGAVK | | | | |
| | | | | | QPSSAYAAFNNRPPSTWLTAY | | | | |
| | | | | | VVK | | | | |
| 18 | CRP_MOUSE | P14847 | C-reactive protein [Precursor] | 62 | TFGHEDMFK | — | — | Crp | Secreted |
| | | | | 4 | TFGHEDMFKK | | | Ptx1 | |
| | | | | 15 | NSNDILIFWNK | | | | |
| | | | | | LLWCLLIMISFSR + O@M | | | | |
| 19 | FETUA_{—} | P29699 | Alpha-2-HS-glycoprotein | 72 | QVLNQIDKVK | — | — | Ahsg | Secreted |
| | MOUSE | | [Precursor] | 7 | GLSDHRTYHDLR | | | Fetua | |
| | | | Fetuin-A | 28 | VGQPGAAGPVSPMCPGR + | | | | |
| | | | Countertrypin | | O@M | | | | |
| | | | | | ANLMHNLGGEEVSVACK + | | | | |
| | | | | | O@M | | | | |
| | | | | | QLTEHAVEGDCDFHILK | | | | |
| | | | | | HAFSPVASVESASGETLHSPK | | | | |
| | | | | | QLTEHAVEGDCDFHILKQDGQF | | | | |
| | | | | | R | | | | |
| 20 | GELS_{—} | P13020 | Gelsolin [Precursor] | 176 | MDAHPPR + O@M | — | — | Gsn | Secreted |
| | MOUSE | | Actin-depolymerizing factor | 19 | LFACSNR | | | Gsb | (Isoform 1) |
| | | | ADF | 31 | RTPITVVR | | | | Cytoplasma |
| | | | Brevin | | EPGLQIWR | | | | (Isoform 2) |
| | | | | | QFFKNWR | | | | |
| | | | | | DGGQTAPASIR | | | | |
| | | | | | YIETDPANR | | | | |
| | | | | | AGKEPGLQIWR | | | | |
| | | | | | HVVPNEVVVQR | | | | |
| | | | | | SEDCFILDHGR | | | | |
| | | | | | EVQGFESSTFSGYFK | | | | |
| | | | | | TPSAAYLWVGAGASEAEK | | | | |
| | | | | | QTQVSVLPEGGETPLFK | | | | |
| | | | | | DPDQTDGPGLGYLSSHIANVER | | | | |
| | | | | | SQHVQVEEGSEPDAFWEALGG | | | | |
| | | | | | K | | | | |
| | | | | | VSNGAGSMSVSLVADENPFAQ | | | | |
| | | | | | GALR | | | | |
| | | | | | VSNGAGSMSVSLVADENPFAQ | | | | |
| | | | | | GALR + O@M | | | | |
| | | | | | FDLVPVPPNLYGDFFTGDAYVI | | | | |
| | | | | | LK | | | | |
| | | | | | VPVDPATYGQFYGGDSYIILYN | | | | |
| | | | | | YR | | | | |
| 21 | GPX3_{—} | P46412 | Glutathione peroxidase 3 | — | — | 40 | YVRPGGGFVPNFQLFE | Gpx3 | Secreted |
| | MOUSE | | [Precursor] | | | 1 | K | | |
| | | | EC 1.11.1.9 | | | 7 | | | |
| | | | GSHPx-3 | | | | | | |
| | | | GPx-3 | | | | | | |
| | | | Plasma glutathione peroxidase | | | | | | |
| | | | GSHPx-P | | | | | | |
| 22 | HA10_{—} | P01898 | H-2 class I histocompatibility | 150 | FDSDAETPR | — | — | H2- | Membrane |
| | MOUSE | | antigen, Q10 alpha chain | 10 | GYLQYAYDGR | | | Q10 | |
| | | | [Precursor] | 31 | GNEQSFHVSLR | | | | |
| | | | | | WEQAGAAEYYR | | | | |
| | | | | | KWEQAGAAEYYR | | | | |
| | | | | | AKGNEQSFHVSLR | | | | |
| | | | | | AYLEAECVEWLLR | | | | |
| | | | | | FIIVGYVDDTQFVR | | | | |
| | | | | | YFETSVSRPGLGEPR | | | | |
| | | | | | THVTHHPGSEGDVTLR | | | | |
| 23 | HBA_MOUSE | P01942 | Hemoglobin subunit alpha | 74 | LRVDPVNFK | — | — | Hba | Extra- |
| | | | Hemoglobin alpha chain | 5 | IGGHGAEYGAEALER | | | Hba- | cellular |
| | | | Alpha-globin | 37 | TYFPHFDVSHGSAQVK | | | a1 | |
| | | | | | TYFPHFDVSHGSAQVKGHGK | | | | |
| | | | | | IGGHGAEYGAEALERMFASFPT | | | | |
| | | | | | TK + O@M | | | | |
| 24 | HEMO_{—} | Q91X72 | Hemopexin [Precursor] | 126 | GPDSVFLIK | 113 | ELGSPPGISLETIDAAF | Hpx | Secreted |
| | MOUSE | | | 14 | YYCFQGNK | 1 | SCPGSSR | Hpxn | |
| | | | | 35 | DYFVSCPGR | 5 | | | |
| | | | | | WFWDFATR | | | | |
| | | | | | NPITSVDAAFR | | | | |
| | | | | | FNPVTGEVPPR | | | | |
| | | | | | KWFWDFATR | | | | |
| | | | | | WKNPITSVDAAFR | | | | |
| | | | | | GATYAFTGSHYWR | | | | |
| | | | | | CSPDPGLTALLSDHR | | | | |
| | | | | | GECQSEGVLFFQGNR | | | | |
| | | | | | SLPQPQKVNSILGCSQ | | | | |
| | | | | | ELGSPPGISLETIDAAFSCPGSS | | | | |
| | | | | | R | | | | |
| | | | | | LFQEEFPGIPYPPDAAVECHR | | | | |
| 25 | IGJ_MOUSE | P01592 | Ig J chain | 89 | CYTTMVPLR + O@M | — | — | Igi | |
| | | | | 6 | NFVYHLSDVCK | | | | |
| | | | | 28 | NFVYHLSDVCKK | | | | |
| | | | | | IIPSTEDPNEDIVER | | | | |
| | | | | | CYTTMVPLRYHGETK + O@M | | | | |
| | | | | | IIPSTEDPNEDIVERNIR | | | | |
| 26 | K2C8_{—} | P11679 | Keratin, type II cytoskeletal 8 | 135 | HGDDLRR | — | — | Krt8 | |
| | MOUSE | | Cytokeratin-8 | 17 | SFTSGPGAR | | | Krt2-8 | |
| | | | CK-8 | 35 | FLEQQNK | | | | |
| | | | Keratin-8 | | VGSSSSSFR | | | | |
| | | | K8 | | QIHEEEIR | | | | |
| | | | Cytokeratin endo A | | AQYEDIANR | | | | |
| | | | | | FASFIDKVR | | | | |
| | | | | | SYKMSTSGPR | | | | |
| | | | | | YEELQTLAGK | | | | |
| | | | | | TKTEISEMNR + O@M | | | | |
| | | | | | AQYEDIANRSR | | | | |
| | | | | | SLDMDGIIAEVR + O@M | | | | |
| | | | | | ASLEAAIADAEQR | | | | |
| | | | | | SRAEAETMYQIK + O@M | | | | |
| | | | | | LESGMQNMSIHTK + 2 O@M | | | | |
| | | | | | DVDEAYMNKVELESR + O@M | | | | |
| | | | | | ELQSQISDTSVVLSMDNSR + | | | | |
| | | | | | O@M | | | | |
| 27 | KAC_MOUSE | P01837 | Ig kappa chain C region | 70 | SFNRNEC | — | — | | |
| | | | | 4 | WKIDGSER | | | | |
| | | | | 32 | HNSYTCEATHK | | | | |
| | | | | | HNSYTCEATHKTSTSPIVK | | | | |
| 28 | KNG1_{—} | O08677 | Kininogen-1 [Precursor] | 75 | YVIEFIAR | 122 | FPSLHGDCVALPNGDD | Kng1 | Secreted |
| | MOUSE | | | 10 | RPPGFSPFR | 1 | GECR | Kng | Extra- |
| | | | | 17 | SGNQYMLHR | 3 | | | cellular |
| | | | | | SGNQYMLHR + O@M | | | | |
| | | | | | ENEFFIVTQTCK | | | | |
| | | | | | DAEEAATGECTATVGK | | | | |
| | | | | | FPSLHGDCVALPNGDDGECR | | | | |
| | | | | | HLGQSLDCNANVYMRPWENK | | | | |
| | | | | | EVLGHSIAQLNAENDHPFYYK | | | | |
| | | | | | HLGQSLDCNANVYMRPWENK + | | | | |
| | | | | | O@M | | | | |
| 29 | KV3B_{—} | P01655 | Ig kappa chain V-III region PC | — | — | 109 | DIVLTQSPASLAVSLGQ | | |
| | MOUSE | | 7132 | | | 1 | R | | |
| | | | | | | 16 | | | |
| 30 | KV5L_{—} | P01645 | Ig kappa chain V-V region HP | 61 | LHSGVPSR | 53 | LLIYYTSR | | |
| | MOUSE | | 93G7 | 5 | LLIYYTSR | 1 | | | |
| | | | | 50 | DIQMTQTTSSLSASLGDR | 7 | | | |
| | | | | | DIQMTQTTSSLSASLGDR + | | | | |
| | | | | | O@M | | | | |
| | | | | | ASQDISNYLNWYQQKPDGTVK | | | | |
| 31 | KV6K_{—} | P04945 | Ig kappa chain V-VI region NQ2- | — | — | 115 | LLIYDTSNLASGVPVR | | |
| | MOUSE | | 6.1 | | | 1 | | | |
| | | | | | | 14 | | | |
| 32 | MBL2_{—} | P41317 | Mannose-binding protein C | 62 | MSLDRVK + O@M | 43 | ALCSEFQGSVATPR | Mbl2 | Rough |
| | MOUSE | | [Precursor] | 5 | DIAYLGITDVR | 1 | | | endoplasmic |
| | | | MBP-C | 24 | VEGSFEDLTGNR | 5 | | | reticulum |
| | | | Mannan-binding protein | | ALCSEFQGSVATPR | | | | membrane; |
| | | | RA-reactive factor P28A subunit | | AEFDTSEIDSEIAALR | | | | Peripheral |
| | | | RARF/P28A | | | | | | membrane |
| | | | | | | | | | protein; |
| | | | | | | | | | Lumenal |
| | | | | | | | | | side. |
| | | | | | | | | | Golgi |
| | | | | | | | | | apparatus |
| | | | | | | | | | membrane; |
| | | | | | | | | | Peripheral |
| | | | | | | | | | membrane |
| | | | | | | | | | protein; |
| | | | | | | | | | Lumenal |
| | | | | | | | | | side |
| 33 | MUC_MOUSE | P01872 | Ig mu chain C region secreted | — | — | 54 | HPPAVYLLPPAR | Igh-6 | Secreted |
| | | | form | | | 1 | | | |
| | | | | | | 2 | | | |
| 34 | MUP2_{—} | P11589 | Major urinary protein 2 | 124 | IEDNGNFR | — | — | Mup2 | Secreted |
| | MOUSE | | [Precursor] | 10 | EKIEDNGNFR | | | | |
| | | | MUP 2 | 50 | ENIIDLSNANR | | | | |
| | | | | | LFLEQIHVLEK | | | | |
| | | | | | FAKLCEEHGILR | | | | |
| | | | | | DGETFQLMGLYGR | | | | |
| | | | | | DGETFQLMGLYGR + O@M | | | | |
| | | | | | INGEWHTIILASDKR | | | | |
| | | | | | AGEYSVTYDGFNTFTIPK | | | | |
| | | | | | IEDNGNFRLFLEQIHVLEK | | | | |
| 35 | MUP8_{—} | P04938 | Major urinary proteins 11 and 8 | 139 | IEDNGNFR | — | — | Mup8 | Secreted |
| | MOUSE | | [Fragment] | 10 | EKIEDNGNFR | | | Mup11 | |
| | | | MUP11 and MUP8 | 72 | ENIIDLSNANR | | | | |
| | | | | | FAQLCEEHGILR | | | | |
| | | | | | DGETFQLMGLYGR | | | | |
| | | | | | DGETFQLMGLYGR + O@M | | | | |
| | | | | | INGEWHTIILASDKR | | | | |
| | | | | | TDYDNFLMAHLINEK + O@M | | | | |
| | | | | | LFLEQIHVLENSLVLK | | | | |
| | | | | | AGEYSVTYDGFNTFTIPK | | | | |
| 36 | MYH9_{—} | Q8VDD5 | Myosin-9 | 54 | VLQRNCAAYLR | — | — | Myh9 | |
| | MOUSE | | Myosin heavy chain 9 | 8 | VDYKADEWLMK | | | | |
| | | | Myosin heavy chain, nonmuscle | 5 | QIATLHAQVTDMK | | | | |
| | | | IIa | | AQQAADKYLYVDK | | | | |
| | | | Nonmuscle myosin heavy chain | | LMATLRNTNPNFVR + O@M | | | | |
| | | | IIa | | VIQYLAHVASSHKSK | | | | |
| | | | NMMHC II-a | | YRFLSNGHVTIPGQQDK | | | | |
| | | | NMMHC-IIA | | KANLQIDQINTDLNLER | | | | |
| | | | Cellular myosin heavy chain, | | | | | | |
| | | | type A | | | | | | |
| | | | Nonmuscle myosin heavy chain- | | | | | | |
| | | | A | | | | | | |
| | | | NMMHC-A | | | | | | |
| 37 | PLF4_{—} | Q9Z126 | Platelet factor 4 [Precursor] | 122 | TISSGIHLK | — | — | Pf4 | Secreted |
| | MOUSE | | PF-4 | 7 | HITSLEVIK | | | | |
| | | | CXCL4 | 44 | HITSLEVIKAGR | | | | |
| | | | | | HCAVPQLIATLK | | | | |
| | | | | | ICLDRQAPLYK | | | | |
| | | | | | AGRHCAVPQLIATLK | | | | |
| | | | | | HCAVPQLIATLKNGR | | | | |
| 38 | PLMN_{—} | P20918 | Plasminogen [Precursor] | 170 | SFQYHSK | — | — | Plg | Secreted |
| | MOUSE | | EC 3.4.21.7 | 21 | LILEPNNR | | | | |
| | | | | 25 | SSRPEFYK | | | | |
| | | | | | DVILFEKR | | | | |
| | | | | | WSEQTPHR | | | | |
| | | | | | WEYCDIPR | | | | |
| | | | | | MRDVILFEK + O@M | | | | |
| | | | | | WEYCNLKR | | | | |
| | | | | | CEGETDFVCR | | | | |
| | | | | | HSIFTPQTNPR | | | | |
| | | | | | VILGAHEEYIR | | | | |
| | | | | | LKEAQLPVIENK | | | | |
| | | | | | TGIGNGYRGTMSR + O@M | | | | |
| | | | | | VCNRVEYLNNR | | | | |
| | | | | | WSEQTPHRHNR | | | | |
| | | | | | GTVSVTVSGKTCQR | | | | |
| | | | | | LILEPNNRDIALLK | | | | |
| | | | | | VIPACLPSPNYMVADR + O@M | | | | |
| | | | | | TAVTAAGTPCQGWAAQEPHR | | | | |
| | | | | | SFQYHSKEQQCVIMAENSK + | | | | |
| | | | | | O@M | | | | |
| | | | | | QLAAGGVSDCLAKCEGETDFV | | | | |
| | | | | | CR | | | | |
| 39 | PROP_{—} | P11680 | Properdin | 89 | TCDHPAPR | — | — | Cfp | Secreted |
| | MOUSE | | | 8 | QRLCTPLLPK | | | Pfc | |
| | | | | 21 | HGGPFCAGDATR | | | | |
| | | | | | MSINCEGTPGQQSR + O@M | | | | |
| | | | | | LRMSINCEGTPGQQSR + O@M | | | | |
| | | | | | HGGPFCAGDATRNQMCNK + | | | | |
| | | | | | O@M | | | | |
| | | | | | CGGHCPGEAQQSQACDTQK | | | | |
| | | | | | SCSAPAPSHQPPGKPCSGPAY | | | | |
| | | | | | EHK | | | | |
| 40 | RETBP_{—} | Q00724 | Plasma retinol-binding protein | 63 | DPNGLSPETR | 98 | LQNLDGTCADSYSFVF | Rbp4 | Secreted |
| | MOUSE | | [Precursor] | 5 | YWGVASFLQR | 1 | SR | | |
| | | | PRBP | 34 | QRQEELCLER | 8 | | | |
| | | | RBP | | LQNLDGTCADSYSFVFSR | | | | |
| | | | | | KDPEGLFLQDNIIAEFSVDEK | | | | |
| 41 | SAMP_{—} | P12246 | Serum amyloid P-component | — | — | 139 | APPSIVLGQEQDNYGG | Apcs | Secreted |
| | MOUSE | | [Precursor] | | | 1 | GFQR | Ptx2 | |
| | | | SAP | | | 8 | | Sap | |
| 42 | SPA3K_{—} | P07759 | Serine protease inhibitor A3K | 135 | VPMMKMK | — | — | Serpin | Secreted |
| | MOUSE | | [Precursor] | 14 | MKLLTTR + O@M | | | a3k | |
| | | | Serpin A3K | 32 | FSIASNYR | | | Mcm2 | |
| | | | Contrapsin | | LSVSQVVHK | | | Spi2 | |
| | | | SPI-2 | | KLSVSQVVHK | | | | |
| | | | | | GKTMEEILEGLK + O@M | | | | |
| | | | | | MQQVEASLQPETLR | | | | |
| | | | | | MQQVEASLQPETLR + O@M | | | | |
| | | | | | MQQVEASLQPETLRK + O@M | | | | |
| | | | | | HFRDEELSCSVLELK | | | | |
| | | | | | AVLDVAETGTEAAAATGVIGGI | | | | |
| | | | | | R | | | | |
| | | | | | ALYQTEAFTADFQQPTEAK | | | | |
| | | | | | AVLDVAETGTEAAAATGVIGGI | | | | |
| | | | | | RK | | | | |
| | | | | | ISFDPQDTFESEFYLDEKR | | | | |
| 43 | TRFE_{—} | Q921l1 | Serotransferrin [Precursor] | 354 | DSAFGLLR | — | — | Tf | Secreted |
| | MOUSE | | Transferrin | 36 | SCHTGLGR | | | Trf | |
| | | | Siderophilin | 51 | VAQEHFGK | | | | |
| | | | Beta-1-metal-binding globulin | | SCHTGVDR | | | | |
| | | | | | IPSHAVVAR | | | | |
| | | | | | TVLPPDGPR | | | | |
| | | | | | GYYAVAVVK | | | | |
| | | | | | LPEGTTPEK | | | | |
| | | | | | KSCHTGLGR | | | | |
| | | | | | VPPRMDYR + O@M | | | | |
| | | | | | DLLFRDDTK | | | | |
| | | | | | WCALSHLER | | | | |
| | | | | | CISFRDHMK | | | | |
| | | | | | LLEACTFH KH | | | | |
| | | | | | EEYNGYTGAFR | | | | |
| | | | | | HTTIFEVLPEK | | | | |
| | | | | | ASDTSITWNNLK | | | | |
| | | | | | WCAVSEHENTK | | | | |
| | | | | | CLVEKGDVAFVK | | | | |
| | | | | | LYLGHNYVTAIR | | | | |
| | | | | | SKDFQLFSSPLGK | | | | |
| | | | | | GTDFQLNQLEGKK | | | | |
| | | | | | DLLFKDSAFGLLR | | | | |
| | | | | | DQYELLCLDNTR | | | | |
| | | | | | QEDFELLCPDGTR | | | | |
| | | | | | YLGAEYMQSVGNMR + 2 O@M | | | | |
| | | | | | KPVDQYEDCYLAR | | | | |
| | | | | | YLGAEYMQSVGNMRK + 2 | | | | |
| | | | | | O@M | | | | |
| | | | | | ADRDQYELLCLDNTR | | | | |
| | | | | | NLKQEDFELLCPDGTR | | | | |
| | | | | | NQQEGVCPEGSIDNSPVK | | | | |
| | | | | | CAPNNKEEYNGYTGAFR | | | | |
| | | | | | DFASCHLAQAPNHVVVSR | | | | |
| | | | | | HQTVLDNTEGKNPAEWAK | | | | |
| | | | | | KPVKDFASCHLAQAPNHVVVS | | | | |
| | | | | | R | | | | |
| | | | | | AVLTSQETLFGGSDCTGNFCLF | | | | |
| | | | | | K | | | | |
| 44 | TTHY_{—} | P07309 | Transthyretin [Precursor] | 79 | FVEGVYR | 167 | TLGISPFHEFADVVFTA | Ttr | Secreted |
| | MOUSE | | Prealbumin | 5 | TSEGSWEPFASGK | 1 | NDSGHR | | |
| | | | | 55 | TAESGELHGLTTDEK | 15 | | | |
| | | | | | TLGISPFHEFADVVFTANDSGH | | | | |
| | | | | | R | | | | |
| | | | | | HYTIAALLSPYSYSTTAVVSNPQ | | | | |
| | | | | | N | | | | |
| 45 | VTDB_{—} | P21614 | Vitamin D-binding protein | 101 | SLSLILYSR | 117 | VPTANLENVLPLAEDFT | Gc | Secreted |
| | MOUSE | | [Precursor] | 9 | HLSLLTTMSNR | 1 | EILSR | | |
| | | | DBP | 30 | RTQVPEVFLSK | 4 | | | |
| | | | Group-specific component | | SCESDAPFPVHPGTPECCTK | | | | |
| | | | Gc-globulin | | DLCGQSTTQAMDQYTFELSR | | | | |
| | | | VDB | | DLCGQSTTQAMDQYTFELSR + | | | | |
| | | | | | O@M | | | | |
| | | | | | VPTANLENVLPLAEDFTEILSR | | | | |
| | | | | | EVVSLTEECCAEGADPTCYDT | | | | |
| | | | | | R | | | | |
| | | | | | LCMAALSHQPQEFPTYVEPTN | | | | |
| | | | | | DEICEAFR | | | | |
| 46 | ZA2G_{—} | Q64726 | Zinc-alpha-2-glycoprotein | 125 | LAFEPER | 39 | CLAYGFYPQR | Azgp1 | Secreted |
| | MOUSE | | [Precursor] | 9 | KLAFEPER | 1 | | | |
| | | | Zn-alpha-2-glycoprotein | 32 | IDPPTVTITSR | 3 | | | |
| | | | Zn-alpha-2-GP | | CLAYGFYPQR | | | | |
| | | | | | AREEIFLVTLK | | | | |
| | | | | | GFSQSLSVQWDR | | | | |
| | | | | | YAYDGEDFIEFNK | | | | |
| | | | | | AYLEEECPEMLKR + O@M | | | | |
| | | | | | FQATAFLNDQAFFHYNSNSGK | | | | |

SUPPLEMENTARY TABLE 2
Investigating the mouse serum proteome

| | | | Chatterji B, Borlak J. A | Chatterji B, Borlak J. Serum |
|---|---|---|---|---|
| | | | 2-DE MALDI-TOF study to | proteomics of lung adenocarcinomas |
| | | | identify disease regulated | induced by targeted overexpression |
| | Swiss- | | serum proteins in lung cancer | of c-raf in alveolar epithelium |
| | Prot | | of c-myc transgenic mice. | identifies candidate biomarkers. |
| | Accession | | Submitted, 2007. | Proteomics, 2007, 7, 3980-91. |
| Swiss-Prot ID | no. | Protein aliases | Total: 46 | Total: 45 |
| A1AG1_MOUSE | Q60590 | Alpha-1-acid glycoprotein 1 | | X |
| | | [Precursor] | | |
| | | AGP 1 | | |
| | | Orosomucoid-1 | | |
| | | OMD 1 | | |
| A1AG8_MUSCR | P21352 | Alpha-1-acid glycoprotein 8 | X | |
| | | [Precursor] | | |
| | | AGP 8 | | |
| | | Orosomucoid-8 | | |
| | | OMD 8 | | |
| A1AT1_MOUSE | P07758 | Alpha-1-antitrypsin 1-1 [Precursor] | X | |
| | | Serine protease inhibitor 1-1 | | |
| | | Alpha-1 protease inhibitor 1 | | |
| | | Alpha-1-antiproteinase | | |
| | | AAT | | |
| A1AT2_MOUSE | P22599 | Alpha-1-antitrypsin 1-2 [Precursor] | | X |
| | | Serine protease inhibitor 1-2 | | |
| | | Alpha-1 protease inhibitor 2 | | |
| | | Alpha-1-antiproteinase | | |
| | | AAT | | |
| A1AT3_MOUSE | Q00896 | Alpha-1-antitrypsin 1-3 [Precursor] | | X |
| | | Serine protease inhibitor 1-3 | | |
| | | Alpha-1 protease inhibitor 3 | | |
| A1AT4_MOUSE | Q00897 | Alpha-1-antitrypsin 1-4 [Precursor] | | X |
| | | Serine protease inhibitor 1-4 | | |
| | | Alpha-1 protease inhibitor 4 | | |
| A1AT6_MOUSE | P81105 | Alpha-1-antitrypsin 1-6 [Precursor] | X | X |
| | | Serine protease inhibitor 1-6 | | |
| | | Alpha-1 protease inhibitor 6 | | |
| A2AP_MOUSE | Q61247 | Alpha-2-antiplasmin [Precursor] | | X |
| | | Alpha-2-plasmin inhibitor | | |
| | | Alpha-2-PI | | |
| | | Alpha-2-AP | | |
| A2MG_MOUSE | Q61838 | Alpha-2-macroglobulin [Precursor] | X | X |
| | | Alpha-2-M | | |
| | | Pregnancy zone protein | | |
| ACTG_MOUSE | P60710 | Actin, cytoplasmic 2 Gamma-actin | X | X |
| AFAM_MOUSE | O89020 | Afamin [Precursor] | X | X |
| | | Alpha-albumin | | |
| | | Alpha-Alb | | |
| ALBU_MOUSE | P07724 | Serum albumin [Precursor] | X | X |
| ANT3_MOUSE | P32261 | Antithrombin-III [Precursor] | X | |
| | | ATIII | | |
| APOA1_MOUSE | Q00623 | Apolipoprotein A-I [Precursor] | X | X |
| | | Apo-AI | | |
| | | ApoA-I | | |
| APOA2_MOUSE | P09813 | Apolipoprotein A-II [Precursor] | | |
| | | Apo-AII | | |
| | | ApoA-II | | |
| APOA4_MOUSE | P06728 | Apolipoprotein A-IV [Precursor] | X | X |
| | | Apo-AIV | | |
| | | ApoA-IV | | |
| APOC3_MOUSE | P33622 | Apolipoprotein C-III [Precursor] | X | |
| | | Apo-CIII | | |
| | | ApoC-III | | |
| APOE_MOUSE | P08226 | Apolipoprotein E [Precursor] | X | X |
| | | Apo-E | | |
| APOH_MOUSE | Q01339 | Beta-2-glycoprotein 1 [Precursor] | X | X |
| | | Beta-2-glycoprotein I | | |
| | | Apolipoprotein H | | |
| | | Apo-H | | |
| | | B2GPI | | |
| | | Beta(2)GPI | | |
| | | Activated protein C-binding protein | | |
| | | APC inhibitor | | |
| CFAB_MOUSE | P04186 | Complement factor B [Precursor] | X | X |
| | | EC 3.4.21.47 | | |
| | | C3/C5 convertase | | |
| CFAH_MOUSE | P06909 | Complement factor H [Precursor] | X | |
| | | Protein beta-1-H | | |
| CLUS_MOUSE | Q06890 | Clusterin [Precursor] | X | X |
| | | Sulfated glycoprotein 2 | | |
| | | SGP-2 | | |
| | | Clustrin | | |
| | | Apolipoprotein J | | |
| | | Apo-J | | |
| CO3_MOUSE | P01027 | Complement C3 [Precursor] | X | |
| | | Synonym HSE-MSF | | |
| CPN2_MOUSE | Q9DBB9 | Carboxypeptidase N subunit 2 | | X |
| | | [Precursor] | | |
| | | Carboxypeptidase N polypeptide 2 | | |
| | | Carboxypeptidase N 83 kDa chain | | |
| | | Carboxypeptidase N regulatory | | |
| | | subunit | | |
| | | Carboxypeptidase N large subunit | | |
| CRP_MOUSE | P14847 | C-reactive protein [Precursor] | X | |
| EGFR_MOUSE | Q01279 | Epidermal growth factor receptor | | X |
| ESTN_MOUSE | P23953 | Liver carboxylesterase N [Precursor] | | X |
| | | EC 3.1.1.1 | | |
| | | PES-N | | |
| | | Lung surfactant convertase | | |
| FCN1_MOUSE | O70165 | Ficolin-1 [Precursor] | | |
| | | Ficolin-A | | |
| | | Ficolin-alpha | | |
| | | M-ficolin | | |
| | | Collagen/fibrinogen domain- | | |
| | | containing protein 1 | | |
| FETUA_MOUSE | P29699 | Alpha-2-HS-glycoprotein [Precursor] | X | X |
| | | Fetuin-A | | |
| | | Countertrypin | | |
| FETUB_MOUSE | Q9QXC1 | Fetuin-B | | X |
| | | IRL685 | | |
| GCB_MOUSE | P01866 | Ig gamma-2B chain C region | | X |
| | | secreted form | | |
| GCBM_MOUSE | P01867 | Ig gamma-2B chain C region, | | X |
| | | membrane-bound form | | |
| GELS_MOUSE | P13020 | Gelsolin [Precursor] | X | X |
| | | Actin-depolymerizing factor | | |
| | | ADF | | |
| | | Brevin | | |
| GPX3_MOUSE | P46412 | Glutathione peroxidase 3 [Precursor] | X | X |
| | | EC 1.11.1.9 | | |
| | | GSHPx-3 | | |
| | | GPx-3 | | |
| | | Plasma glutathione peroxidase | | |
| | | GSHPx-P | | |
| HA10_MOUSE | P01898 | H-2 class I histocompatibility | X | X |
| | | antigen, | | |
| | | Q10 alpha chain [Precursor] | | |
| HBA_MOUSE | P01942 | Hemoglobin subunit alpha | X | X |
| | | Hemoglobin alpha chain | | |
| | | Alpha-globin | | |
| HEMO_MOUSE | Q91X72 | Hemopexin [Precursor] | X | X |
| HPT_MOUSE | Q62558 | Haptoglobin | | |
| HV51_MOUSE | P06330 | Ig heavy chain V region AC38 | | X |
| | | 205.12 | | |
| IGHG1_MOUSE | P01868 | Ig gamma-1 chain C region secreted | | X |
| | | form | | |
| IGJ_MOUSE | P01592 | Ig J chain | X | X |
| K2C8_MOUSE | P11679 | Keratin, type II cytoskeletal 8 | X | |
| | | Cytokeratin-8 | | |
| | | CK-8 | | |
| | | Keratin-8 | | |
| | | K8 | | |
| | | Cytokeratin endo A | | |
| KAC_MOUSE | P01837 | Ig kappa chain C region | X | X |
| KBP_MOUSE | P29621 | Serine protease inhibitor A3C | | |
| | | [Precursor] | | |
| | | Serpin A3C | | |
| | | Kallikrein-binding protein | | |
| | | KBP | | |
| KNG1_MOUSE | O08677 | Kininogen-1 [Precursor] | X | X |
| KV3C_MOUSE | P01656 | Ig kappa chain V-III region MOPC 70 | | X |
| KV3D_MOUSE | P03977 | Ig kappa chain V-III region 50S10.1 | | X |
| KV5K_MOUSE | P01644 | Ig kappa chain V-V region HP R16.7 | | X |
| KV5L_MOUSE | P01645 | Ig kappa chain V-V region HP 93G7 | X | X |
| KV6K_MOUSE | P04945 | Ig kappa chain V-VI region NQ2-6.1 | X | |
| LIFR_MOUSE | P42703 | Leukemia inhibitory factor receptor | | |
| | | [Precursor] | | |
| | | LIF receptor | | |
| | | LIF-R | | |
| | | D-factor/LIF receptor | | |
| | | CD118 antigen | | |
| MBL1_MOUSE | P39039 | Mannose-binding protein A | | X |
| | | [Precursor] | | |
| | | MBP-A | | |
| | | Mannan-binding protein | | |
| | | Ra-reactive factor polysaccharide- | | |
| | | binding component p28B | | |
| | | polypeptide | | |
| | | RaRF p28B | | |
| MBL2_MOUSE | P41317 | Mannose-binding protein C | X | |
| | | [Precursor] | | |
| | | MBP-C | | |
| | | Mannan-binding protein | | |
| | | RA-reactive factor P28A subunit | | |
| | | RARF/P28A | | |
| MUC_MOUSE | P01872 | Ig mu chain C region secreted form | X | X |
| MUP1_MOUSE | P11588 | Major urinary protein 1 [Precursor] | | |
| | | MUP 1 | | |
| MUP2_MOUSE | P11589 | Major urinary protein 2 [Precursor] | X | |
| | | MUP 2 | | |
| MUP4_MOUSE | P11590 | Major urinary protein 4 [Precursor] | | |
| | | MUP 4 | | |
| MUP6_MOUSE | P02762 | Major urinary protein 6 [Precursor] | | |
| | | MUP 6 | | |
| | | Alpha-2U-globulin | | |
| | | Group 1, BS6 | | |
| | | Allergen Mus m 1 | | |
| MUP8_MOUSE | P04938 | Major urinary proteins 11 and 8 | X | X |
| | | [Fragment] | | |
| | | MUP11 and MUP8 | | |
| MYH9_MOUSE | Q8VDD5 | Myosin-9 | X | |
| | | Myosin heavy chain 9 | | |
| | | Myosin heavy chain, nonmuscle IIa | | |
| | | Nonmuscle myosin heavy chain IIa | | |
| | | NMMHC II-a | | |
| | | NMMHC-IIA | | |
| | | Cellular myosin heavy chain, type A | | |
| | | Nonmuscle myosin heavy chain-A | | |
| | | NMMHC-A | | |
| PLF4_MOUSE | Q9Z126 | Platelet factor 4 [Precursor] | X | |
| | | PF-4 | | |
| | | CXCL4 | | |
| PLMN_MOUSE | P20918 | Plasminogen [Precursor] | X | X |
| | | EC 3.4.21.7 | | |
| PROP_MOUSE | P11680 | Properdin | X | X |
| Q91XL1_MOUSE | Q91XL1 | Leucine-rich alpha-2-glycoprotein 1 | | |
| | | Leucine-rich HEV glycoprotein | | |
| RETBP_MOUSE | Q00724 | Plasma retinol-binding protein | X | X |
| | | [Precursor] | | |
| | | PRBP | | |
| | | RBP | | |
| S6A11_MOUSE | P31650 | Sodium- and chloride-dependent | | X |
| | | GABA transporter 4 | | |
| | | GAT4 | | |
| SAA1_MOUSE | P05366 | Serum amyloid A-1 protein | | |
| | | [Precursor] | | |
| SAA2_MOUSE | P05367 | Serum amyloid A-2 protein | | |
| | | [Precursor] | | |
| SAA4_MOUSE | P31532 | Serum amyloid A-4 protein | | X |
| | | [Precursor] | | |
| | | Amyloid A-5 protein | | |
| SAMP_MOUSE | P12246 | Serum amyloid P-component | X | |
| | | [Precursor] | | |
| | | SAP | | |
| SPA3K_MOUSE | P07759 | Serine protease inhibitor A3K | X | |
| | | [Precursor] | | |
| | | Serpin A3K | | |
| | | Contrapsin | | |
| | | SPI-2 | | |
| TRFE_MOUSE | Q921I1 | Serotransferrin [Precursor] | X | X |
| | | Transferrin | | |
| | | Siderophilin | | |
| | | Beta-1-metal-binding globulin | | |
| TTHY_MOUSE | P07309 | Transthyretin [Precursor] | X | X |
| | | Prealbumin | | |
| THRB_MOUSE | P19221 | Prothrombin [Precursor] | | |
| | | EC 3.4.21.5 | | |
| | | Coagulation factor II | | |
| VTDB_MOUSE | P21614 | Vitamin D-binding protein | X | X |
| | | [Precursor] | | |
| | | DBP | | |
| | | Group-specific component | | |
| | | Gc-globulin | | |
| | | VDB | | |
| VTNC_MOUSE | P29788 | Vitronectin [Precursor] | | X |
| | | Serum-spreading factor | | |
| | | S-protein | | |
| ZA2G_MOUSE | Q64726 | Zinc-alpha-2-glycoprotein | X | |
| | | [Precursor] | | |
| | | Zn-alpha-2-glycoprotein | | |
| | | Zn-alpha-2-GP | | |
| ZA2G_MOUSE | Q64726 | Zinc-alpha-2-glycoprotein | | X |
| | | [Precursor] | | |
| | | Zn-alpha-2-glycoprotein | | |
| | | Zn-alpha-2-GP | | |

| | Wait R et al. Reference maps | |
|---|---|---|
| | of mouse serum acute-phase | Duan X et al. A mouse serum |
| | proteins: changes with LPS- | two-dimensional gel map: |
| | induced inflammation and | application to profiling burn |
| | apolipoprotein A-I and A- | injury and infection. |
| | II transgenes. Proteomics. | Electrophoresis. 2004, |
| | 2005, 5, 4245-53. | 25, 3055-65. |
| Swiss-Prot ID | Total: 30 | Total: 38 |
| A1AG1_MOUSE | X | X |
| A1AG8_MUSCR | | |
| A1AT1_MOUSE | X | X |
| A1AT2_MOUSE | X | X |
| A1AT3_MOUSE | X | X |
| A1AT4_MOUSE | | |
| A1AT6_MOUSE | | X |
| A2AP_MOUSE | | |
| A2MG_MOUSE | | X |
| ACTG_MOUSE | | |
| AFAM_MOUSE | X | |
| ALBU_MOUSE | X | X |
| ANT3_MOUSE | | |
| APOA1_MOUSE | X | X |
| APOA2_MOUSE | X | X |
| APOA4_MOUSE | X | X |
| APOC3_MOUSE | X | X |
| APOE_MOUSE | X | X |
| APOH_MOUSE | | X |
| CFAB_MOUSE | | |
| CFAH_MOUSE | | X |
| CLUS_MOUSE | X | X |
| CO3_MOUSE | | |
| CPN2_MOUSE | | |
| CRP_MOUSE | | |
| EGFR_MOUSE | | X |
| ESTN_MOUSE | X | |
| FCN1_MOUSE | | X |
| FETUA_MOUSE | X | X |
| FETUB_MOUSE | X | X |
| GCB_MOUSE | | |
| GCBM_MOUSE | | |
| GELS_MOUSE | | X |
| GPX3_MOUSE | | |
| HA10_MOUSE | | |
| HBA_MOUSE | | |
| HEMO_MOUSE | X | X |
| HPT_MOUSE | X | X |
| HV51_MOUSE | | |
| IGHG1_MOUSE | | |
| IGJ_MOUSE | | |
| K2C8_MOUSE | | |
| KAC_MOUSE | | |
| KBP_MOUSE | X | |
| KNG1_MOUSE | X | X |
| KV3C_MOUSE | | |
| KV3D_MOUSE | | |
| KV5K_MOUSE | | |
| KV5L_MOUSE | | |
| KV6K_MOUSE | | |
| LIFR_MOUSE | | X |
| MBL1_MOUSE | | |
| MBL2_MOUSE | | X |
| MUC_MOUSE | | |
| MUP1_MOUSE | X | |
| MUP2_MOUSE | X | X |
| MUP4_MOUSE | X | |
| MUP6_MOUSE | X | |
| MUP8_MOUSE | X | |
| MYH9_MOUSE | | |
| PLF4_MOUSE | | |
| PLMN_MOUSE | | X |
| PROP_MOUSE | | |
| Q91XL1_MOUSE | X | |
| RETBP_MOUSE | | X |
| S6A11_MOUSE | | |
| SAA1_MOUSE | | X |
| SAA2_MOUSE | | X |
| SAA4_MOUSE | | |
| SAMP_MOUSE | X | X |
| SPA3K_MOUSE | X | X |
| TRFE_MOUSE | X | X |
| TTHY_MOUSE | X | X |
| THRB_MOUSE | | X |
| VTDB_MOUSE | X | X |
| VTNC_MOUSE | | |
| ZA2G_MOUSE | | X |
| ZA2G_MOUSE | | |

We previously reported targeted overexpression of c-myc to alveolar epithelium to cause lung cancer. We now extended our studies to the serum proteome of tumor bearing mice (Chatterji B, Borlak J. Proteomics. 2009 February; 9(4):1044-56). Proteins were extracted with a thiourea-containing lysis buffer and separated by 2-DE at pH 4-7 and 3-10 followed by MALDI-TOF/TOF analysis. Fourty six proteins were identified in tumor bearing mice of which n=9 were statistically significant. This included disease regulated expression of orosomucoid-8, alpha-2-macroglobulin, apolipoprotein-A1, apolipoprotein-C3, glutathione peroxidase-3, plasma retinol-binding protein and transthyretin, while expression of apolipoprotein-E was decreased in late stages of disease. Moreover, serum amyloid P component was uniquely expressed at late stages of cancer. It is of considerable importance that most disease regulated proteins carried the E-Box sequence (CACGTG) in the promoter of the coding gene, therefore providing evidence for their regulation by c-myc. Notably, expression of alpha-2-macroglobulin, transthyretin, alpha-1-antitrypsin and properdin was in common in different lung tumor models, but regulation of orosomucoid-8, apolipoprotein-Al, apolipoprotein-C3, apolipoprotein-E, glutathione peroxidase-3, plasma retinol-binding protein and serum amyloid P component was unique when the serum proteome of c-myc and c-raf tumor bearing mice were compared. Therefore, candidate biomarkers for atypical adenomatous hyperplasias (AAH) and bronchiolo-alveolar (BAC)/papillary adenocarcinomas (PLAC) can be proposed.

Patents, patent applications and scientific articles referenced in this document are incorporated by reference herein. No admission is made that any of these documents qualify as prior art for any purpose.

What is claimed is:

1. Use of at least one biomarker selected from the group consisting of APOE, APOC3, A1AT6, A2MG, PROP, TTHY, A1AG8, APOA1, APOH, GPX3, MUP8, RETBP, SAMP, VTDB, S6A11, EGFR, ApoA4, ApoM, a-raf, fetuin B, GSN, PLG, VPS28, or of at least one antibody directed against said at least one biomarker, in the diagnosis, prognosis and/or treatment monitoring of cancer or dysplasia, in particular of lung cancer or bronchial dysplasia.

2. Use as claimed in claim 1 for monitoring the therapeutic treatment of a patient suffering from lung cancer or having bronchial dysplasia, in particular the treatment with a chemotherapeutic agent, preferably with an antineoplastic chemotherapy drug, or with a chemopreventive drug.

3. Use as claimed in claim 1, wherein the at least one biomarker is selected from the group consisting of
APOE, APOC3, A1AG8, APOA1, APOH, GPX3, RETBP, SAMP in particular for the diagnosis, prognosis and/or treatment monitoring of BAC and/or
wherein the at least one biomarker is selected from the group consisting of MUP8, VTDB, S6A11, EGFR in particular for the diagnosis, prognosis and/or treatment monitoring of AAH and/or wherein the at least one biomarker is selected from the group consisting of ApoA4, ApoM, a-raf, fetuin B, GSN, PLG, VPS28 in particular for the diagnosis, prognosis and/or treatment monitoring of bronchial dysplasia or lung cancer.

4. Use as claimed in claim 1, wherein at least one biomarker selected from the group consisting of APOE, APOC3, A1AG8, APOA1, APOH, GPX3, RETBP, SAMP and at least one biomarker selected from the group consisiting of MUP8, VTDB, S6A11, EGFR are used.

5. Use as claimed in claim 1, comprising
(a) measuring the level of at least one biomarker selected from the group consisting of APOE, APOC3, A1AT6, A2MG, PROP, TTHY, A1AG8, APOA1, APOH, GPX3, MUP8 RETBP, SAMP, VTDB, S6A11, EGFR, ApoA4, ApoM, a-raf, fetuin B, GSN, PLG, VPS28 in a body fluid sample, in particular in a blood serum sample, of a patient suffering from or being susceptible to cancer, and
(b) comparing the level of said at least one biomarker in said sample to a reference level of said at least one biomarker, in particular by the use according to claim 1 in particular
for monitoring the therapeutic treatment of a patient suffering from lung cancer or having bronchial dysplasia, in particular the treatment with a chemotherapeutic agent, preferably with an antineoplastic chemotherapy drug, or with a chemopreventive drug.

6. Use as claimed in claim 5, wherein the at least one biomarker is selected from the group consisting of
APOE, APOC3, A1AG8, APOA1, APOH, GPX3, RETBP, SAMP in particular for the diagnosis, prognosis, staging and/or treatment monitoring of BAC and/or wherein the at least one biomarker is selected from the group consisting of MUP8, VTDB, S6A11, EGFR. In particular for the diagnosis, prognosis, staging and/or treatment monitoring of AAH and/or wherein the at least one biomarker is selected from the group consisting of
ApoA4, ApoM, a-raf, fetuin B, GSN, PLG, VPS28 in particularfor the diagnosis, prognosis, staging and/or treatment monitoring of dysplasia or cancer, in particular of bronchial dysplasia or lung cancer.

7. Use as claimed in claim 5 to distinguish between different subtypes of lung cancer, such as (but not limited to) lung adenocarcinomas as defined by AAH or BAC, wherein at least one biomarker selected from the group consisting of APOE, APOC3, A1AG8, APOA1, APOH, GPX3, RETBP, SAMP and at least one biomarker selected from the group consisting of MUP8, VTDB, S6A11, EGFR are measured.

8. A biomarker,
a)
preferably for use in diagnosing or treatment monitoring of lung cancer, selected from a first group consisting of
APOC3, A1AT6, A2MG, PROP, TTHY, A1AG8, APOA1, APOH, GPX3, MUP8, RETBP, SAMP, VTDB, S6A11, EGFR
or from a second group consisting of
APOE and sequence fragments thereof, in particular sequence fragments of APOE being 6-24 amino acid residues in length,
wherein the biomarker is regulated by c-myc overexpression in a subject, or antibody directed against said biomarker, preferably for use in diagnosing or treatment monitoring of lung cancer, in particular bronchial dysplasia or lung cancer and/or
b)
preferably for use in diagnosing or treatment monitoring of dysplasia or lung cancer, in particular bronchial dysplasia or lung cancer, selected from the group consisiting of
ApoA4, ApoM, a-raf, fetuin B, GSN, PLG, VPS28,
wherein the biomarker is regulated by aberrant EGF receptor tyrosine kinase signaling in a subject,
or antibody directed against said biomarker, preferably for use in diagnosing or treatment monitoring of lung cancer, in particular bronchial dysplasia or lung cancer and/or
c)
for qualifying the c-myc activity in a patient suffering from or being susceptible to cancer or for classifying a patient suffering from or being susceptible to lung cancer selected from a first group consisting of sequence fragments of the group of biomarkers according to claim 8 a) or from a second group consisting of sequence fragments of APOE,
wherein the sequence fragments are 6-24 amino acid residues in length and are preferably synthetic peptides and/or d) for diagnosing or treatment monitoring of dysplasia or cancer, in particular of bronchial dysplasia or lung cancer, associated with aberrant EGF receptor tyrosine kinase signaling in a patient, selected from a group consisting of sequence fragments of the group of biomarkers according to claim 8 b),
wherein the sequence fragments are 6-24 amino acid residues in length and are preferably synthetic peptides.

9. Biomarker as claimed in one claim 8 a, c selected from a first group consisting of
LAQIHFPR (SEQ ID NO: 1), TLMSPLGITR (SEQ ID NO: 2), RLAQIHFPR (SEQ ID NO: 3), MQHLEQTLSK (SEQ ID NO: 4), ELISKFLLNR (SEQ ID NO: 5), IFNNGADLSGITEENAPLK (SEQ ID NO: 6), NHYQAEVFSVNFAESEEAK (SEQ ID NO: 7), DQSPASHEIATNLGDFAISLYR (SEQ ID NO: 8), KPFDPENTEEAEFHVDESTTVK (SEQ ID NO: 9), FDHPFLFIIFEEHTQSPLFVGK (SEQ ID NO: 10), APFALQVNTLPLNFDK (SEQ ID NO: 11), TCDHPAPR (SEQ ID NO: 12), QRLCTPLLPK (SEQ ID NO: 13), HGGPFCAGDATR (SEQ ID NO: 14), MSINCEGTPGQQSR (SEQ ID NO: 15), LRMSINCEGTPGQQSR (SEQ ID NO: 16), HGGPFCAGDATRNQMCMK (SEQ ID NO: 17), CGGHCPGEAQQSQACDTQK (SEQ ID NO: 18), SCSAPAPSHQPPGKPCSGPAYEHK (SEQ ID NO: 19), FVEGVYR (SEQ ID NO: 20), TSEGSWEPFASGK (SEQ ID NO: 21), TAESGELHGLTTDEK (SEQ ID NO: 22), TLGISPFHEFADVVFTANDSGHR (SEQ ID NO: 23), HYTIAALLSPYSYSTTAVVSNPQN (SEQ ID NO: 24), YEGGVETFAHLIVLR (SEQ ID NO: 25). LQELQGR (SEQ ID NO: 26), EDVELYR (SEQ ID NO: 27), ARPALEDLR (SEQ ID NO: 28), LSPVAEEFR (SEQ ID NO: 29), QKLQELQGR (SEQ ID NO: 30), WKEDVELYR (SEQ ID NO: 31), VQPYLDEFQK (SEQ ID NO: 32), TQLAPHSEQMR (SEQ ID NO. 33), LSPVAEEFRDR (SEQ ID NO: 34), SNPTLNEYHTR (SEQ ID NO: 35), VAPLGAELQESAR (SEQ ID NO: 36), QEMNKDLEEVK (SEQ ID NO: 37), VKDFANVYVDAVK (SEQ ID NO: 38), LQELQGRLSPVAEEFR (SEQ ID NO: 39), TVQDALSSVQESDIAVVAR (SEQ ID NO: 40), IHFYCK (SEQ ID NO: 41), ATVLYQGMR (SEQ ID NO: 42), ITCPPPVPK (SEQ ID NO: 43), WSPDIPACAR (SEQ ID NO: 44), DGTIEIPSCFK (SEQ ID NO: 45), CSYTVAEHCR (SEQ ID NO: 46), TGTWSFLPTCR (SEQ ID NO: 47), VCPFAGILENGIVR (SEQ ID NO: 48), IQEQFKNGMMHGDK (SEQ ID NO: 49), FTCPLTGMWPINTLR (SEQ ID NO: 50), ICPKPDDLPFATVVPLK (SEQ ID NO: 51), TSYDPGEQIVYSCKPGYVSR (SEQ ID NO: 52), CPFPPRPENGYVNYPAKPVLLYK (SEQ ID NO: 53), YVRPGGFVPNFQLFEK (SEQ ID NO: 54), IEDNGNFR (SEQ ID NO: 107), EKIEDNGNFR (SEQ ID NO: 108), ENIIDLSNANR (SEQ ID NO: 109), FAQLCEEHGILR (SEQ ID NO: 110), DGETFQLMGLYGR (SEQ ID NO: 111), INGEWHTIILASDKAR (SEQ ID NO. 112), TDYDNFLMAHLINEK (SEQ ID NO: 113), LFLEQIHVLENSLVLK (SEQ ID NO: 114), AGEYSVTYDGFNTFTIPK (SEQ ID NO: 115), DPNGLSPETR (SEQ ID NO: 55), YWGVASFLQR (SEQ ID NO: 56), QRQEELCLER (SEQ ID NO: 57), LQNLDGTCADSYSFVFSR (SEQ ID NO: 58), KDPEGLFLQDNIIAEFSVDEK (SEQ ID NO: 59), APPSIVLGQEQDNYGGGFQR (SEQ ID NO: 60),
or from a second group consisting of
EVQAAQAR (SEQ ID NO: 61), FWDYLR (SEQ ID NO: 62), DRLEEVR (SEQ ID NO: 63), EHMEEVR (SEQ ID NO: 64), LGPLVEQGR (SEQ ID NO: 65), LEEVGNQAR (SEQ ID NO: 66), QWANLMEK (SEQ ID NO: 67), DRAQAFGDR (SEQ ID NO: 68), LQAEIFQAR (SEQ ID NO: 69), MEEQTQQIR (SEQ ID NO: 70), GRLEEVGNQAR (SEQ ID NO: 71), TANLGAGAAQPLR (SEQ ID NO: 72), SKMEEQTQQIR (SEQ ID NO: 73), GWFEPIVEDMHR (SEQ ID NO: 74), ELEEQLGPVAEETR (SEQ ID NO: 75), NEVHTMLGQSTEEIR (SEQ ID NO: 76)
or from a third group, consisting of
GNTEGLQK (SEQ ID NO: 116), LQLTPYIQR (SEQ ID NO: 117), ALVQQLEQFR (SEQ ID NO: 118), QLEQQVEEFR (SEQ ID NO: 119), ATIDQNLEDLRR (SEQ ID NO: 120), QLEQQVEEFRR (SEQ ID NO: 121), LNHQMEGLAFQMK (SEQ ID NO: 122), TDVTQQLSTLFQDK (SEQ ID NO: 123), LVPFVVQLSGHLAKETER (SEQ ID NO: 124), QQLGPNSGEVESHLSFLEK (SEQ ID NO: 125), LQEHLKPYAVDLQDQINTQTQEMK (SEQ ID NO: 126), LVPFVVQLSGHLAKETER (SEQ ID NO: 127), AFLVTPR (SEQ ID NO: 128), FLLYNR (SEQ ID NO: 129), ETGQGYQR (SEQ ID NO: 130), SPHSKLPSEQR (SEQ ID NO: 131), LVVLPFPGK (SEQ ID NO: 132), DGYMLSLNR (SEQ ID NO: 133), AMFHINKPR (SEQ ID NO: 134), AMNQWVSGPAYYVEYLIK (SEQ ID NO: 135), SQASCSLQHSDSEPVGICQGSTVQSSLR (SEQ ID NO: 136), EHYQEDMGSLFYLTLDVLETDCHVLSR (SEQ ID NO: 137), LFACSNR (SEQ ID NO: 138), RTPITVVR (SEQ ID NO: 139), EPGLQIWR (SEQ ID NO: 140), DGGQTAPASIR (SEQ ID NO: 141), AGKEPGLQIWR (SEQ ID NO: 142), HVVPNEVVVQR (SEQ ID NO: 143), SEDCFILDHGR (SEQ ID NO: 144), EVQGFESSTFSGYFK (SEQ ID NO: 145), QTQVSVLPEGGETPLFK (SEQ ID NO: 146), EPAHLMSLFGGKPMIIYK (SEQ ID NO: 147), SQHVQVEEGSEPDAFWEALGGK (SEQ ID NO: 148), VSNGAGSMSVSLVADENPFAQGALR (SEQ ID NO: 149), VPVDPATYGQFYGGDSYIILYNYR (SEQ ID NO: 150), NWRDPDQTDGPGLGYLSSHIANVER (SEQ ID NO: 151), IEGSNKVPVDPATYGQFYGGDSYIILYNYR (SEQ ID NO: 152), HVVPNEVVVQR (SEQ ID NO: 153), FVDWIER (SEQ ID NO: 154), LILEPNNR (SEQ ID NO: 155), SSRPEFYK (SEQ ID NO: 156), WSEQTPHR (SEQ ID NO: 157), NLEENYCR (SEQ ID NO: 158), WEYCDIPR (SEQ ID NO: 159), MRDVILFEK (SEQ ID NO: 160), WEYCNLKR (SEQ ID NO: 161), CEGETDFVCR (SEQ ID NO: 162), HSIFTPQTNPR (SEQ ID NO: 163), VILGAHEEYIR (SEQ ID NO: 164), CQSWAAMFPHR (SEQ ID NO: 165), VCNRVEYLNNR (SEQ ID NO: 166), GPWCYTTDPSVR (SEQ ID NO: 167), GTVSVTVSGKTCQR (SEQ ID NO: 168), DIALLKLSRPATITDK (SEQ ID NO: 169), VIPACLPSPNYMVADR (SEQ ID NO: 170), CTTPPPPPSPTYQCLK (SEQ ID NO: 171), TPENFPDAGLEMNYCR (SEQ ID NO: 172), NPDGDVNGPWCYTTNPR (SEQ ID NO: 173), NPDGEPRPWCFTTDPTK (SEQ ID NO: 174), NPDGDKGPWCYTTDPSVR (SEQ ID NO: 175), TICYITGWGETQGTFGAGR (SEQ ID NO: 176), TAVTAAGTPCQGWAAQEPHR (SEQ ID NO: 177), NPDGETAPWCYTTDSQLR (SEQ ID NO: 178), VVGGCVANPHSWPWQISLR (SEQ ID NO: 179), NPDGEPRPWCFTTDPTKR (SEQ ID NO: 180), NPDNDEQGPWCYTTDPDKR (SEQ ID NO: 181), YILQGVTSWGLGCARPNKPGVYVR (SEQ ID NO: 182), FTGQHFCGGTLIAPEWVLTAAHCLEK (SEQ ID NO: 183), WGATFPHVPNYSPSTHPNEGLEENYCR (SEQ ID NO: 184), LEIRAMDEIQPDLR (SEQ ID NO: 185), LEIRAMDEIQPDLR (SEQ ID NO: 186), QVQGSEISSIDEFCRK (SEQ ID NO: 187), YDNMAELFAVVKTMQALEK (SEQ ID NO: 188), AMDEIQPDLRELMETMHR (SEQ ID NO: 189), QTVSQWLQTLSGMSASDELDDSQVR (SEQ ID NO: 190), FHGIPATPGVGAPGNKPELYEEVKLYK (SEQ ID NO: 191)
and wherein, if applicable, at least one amino acid methionine is preferably oxidized according to the Supplementary tables 1.

10. A composition
a)
for qualifying the c-myc activity in a patient suffering or being susceptible to cancer or for classifying a patient suffering from or being susceptible to lung cancer, in particular by an in vitro body fluid analysis, comprising an effective amount of at least one biomarker selected from the first group according to one of the claims 8 a), c), 9 and/or an effective amount of at least one biomarker selected from the second group according to one of the claim 8 a), c), or 9, or comprising at least one antibody directed against said at least one biomarker, in particular for use in qualifying the c-myc activity in a patient suffering from or being susceptible to cancer or for use in classifying a patient suffering from or being susceptible to lung cancer and/or a b) for diagnosing or treatment monitoring of dysplasia or cancer, in particular of bronchial dysplasia or lung cancer, associated with aberrant EGF receptor tyrosine kinase signaling in a patient, preferably by an in vitro body fluid analysis, comprising an effective amount of at least one biomarker selected from the group according to claim 8 b), d) or an effective amount of at least one biomarker selected from the third group according to claim 9, or comprising at least one antibody directed against said at least one biomarker, in particular for use in diagnosing or treatment monitoring of dysplasia or cancer, preferably of bronchial dysplasia or lung cancer, associated with aberrant EGF receptor tyrosine kinase signaling in a patient.

11. Use of a composition as claimed in claim 10 a) for the production of a diagnostic agent, in particular of a diagnostic standard for body fluid analysis and/or for qualifying the c-myc activity in a patient suffering or being susceptible to cancer or for classifying a patient suffering from or being susceptible to lung cancer and/or for predicting or monitoring the response of a cancer patient to a method of treating cancer comprising administering a c-myc activity modulator, e.g. an inhibitor of c-myc/max dimerization.
and/or as claimed in claim 10 b)
for the production of a diagnostic agent, in particular of a diagnostic standard for body fluid analysis, preferably for predicting or monitoring the response of a dysplasia or cancer patient, in particular having bronchial dysplasia or lung cancer, to a method of treating dysplasia comprising administering a chemopreventive drug, such as Zileuton may be or to a method treating cancer comprising administering an EGF receptor tyrosine kinase activity modulator, such as Gefitinib and/or Erlotinib.

12. A composition as claimed in claim 10 a) for qualifying the the c-myc activity in a patient suffering or being susceptible to cancer or for classifying a patient suffering from or being susceptible to lung cancer, in particular for predicting or monitoring the response of a cancer patient to a method of treating cancer comprising administering an c-myc activity modulator, comprising at least one standard (1) indicative of the body fluid level of a biomarker selected from the first group according to one of the claims 8 a, c, 9 in normal individuals or individuals having cancer associated with increased c-myc activity and/or at least one standard (2) indicative of the body fluid level of a biomarker selected from the second group according to one of the claims, 8 a), c), 9 in normal individuals or individuals having cancer associated with increased c-myc activity, and/or comprising at least one antibody directed against said at least one biomarker, in particular for use in qualifying the c-myc activity in a patient suffering or being susceptible to cancer or for use in classifying a patient suffering from or being susceptible to lung cancer, and instructions for the use of the kit.

13. A composition as claimed in claim 10 b) for diagnosing or treatment monitoring of dysplasia or cancer, in particular of bronchial dysplasia or lung cancer, associated with aberrant EGF receptor tyrosine kinase signaling in a patient, in particular for predicting or monitoring the response of the dysplasia patient or the cancer patient to a method of treating dysplasia by administering a chemopreventive drug, such as Zileuton may be, or to a method of treating cancer by administering an EGF receptor tyrosine kinase activity modulator, such as Gefitinib and/or Erlotinib may be, comprising at least one standard indicative of the body fluid level of a biomarker selected from the group according to one of the claim 8 b) or 8 d) or according to the third group according to claim 9 in normal individuals or individuals having dysplasia or cancer related to aberrant EGF receptor tyrosine kinase signaling, and/or comprising at least one antibody directed against said at least one biomarker, in particular for use in diagnosing or treatment monitoring of dysplasia or cancer, preferably of bronchial dysplasia or lung cancer, associated with aberrant EGF receptor tyrosine kinase signaling in a patient, and instructions for the use of the kit.

14. Use as claimed in claim 1 for qualifying the c-myc activity in a patient suffering or being susceptible to cancer or for classifying a patient suffering from or being susceptible to lung cancer, comprising determining in a body fluid sample of a subject suffering from or being susceptible to cancer at least one biomarker selected from the first group according to one of the claims 8 a), c), 9 and/or at least one biomarker selected from the second group according to one of the claims 8 a), c), 9 wherein the body fluid level of the at least one biomarker of said first group being significantly higher and/or the body fluid level of the at least one biomarker of said second group being significantly lower than the level of said biomarker(s) in the body fluid of subjects without cancer associated with increased activity of c-myc is indicative of induced c-myc kinase activity in the subject.

15. Use as claimed in claim 1 of diagnosing or treatment monitoring of dysplasia or cancer, in particular of bronchial dysplasia or lung cancer, associated with aberrant EGF receptor tyrosine kinase signaling in a patient, in particular for predicting or monitoring the response of the dysplasia patient or the cancer patient to a method of treating dysplasia by administering a chemopreventive drug, such as Zileuton may be, or to a method of treating cancer by administering an EGF receptor tyrosine kinase activity modulator, such as Gefitinib and/or Erlotinib may be, comprising determining in a body fluid sample of a subject having or being susceptible to dysplasia at least one biomarker selected from the group of fetuin B, GSN, VPS28 or their fragments according to one of the claims 8 b, d, 9 and/or at least one biomarker selected from the group of ApoA4, ApoM, a-raf, PLG or their fragments according to one of the claims 8 b, d, 9, wherein the body fluid level of the at least one biomarker of the group of fetuin B, GSN, VPS28 or their fragments being significantly higher and/or the body fluid level of the at least one biomarker related to the group of ApoA4, ApoM, a-raf, PLG or their fragments being significantly lower than the level of said biomarker(s) in the body fluid of subjects without dysplasia or cancer associated with aberrant EGF receptor tyrosine kinase signaling, is indicative of aberrant EGF receptor tyrosine kinase signaling in the subject.

16. Use as claimed in claim 13, wherein an immunoassay is performed.

17. Use as claimed in claim 14, wherein an immunoassay is performed.

18. Use as claimed in claim 13, wherein a peptide mass fingerprinting is performed, in particular comprising the steps of
isolating a serum sample from a blood sample of a subject suffering from or being susceptible to cancer;
adding lysis buffer to the serum sample;
separating the proteins of the lysed serum sample by 2-D gel electrophoresis;
excising from the gel at least one sample containing a protein of interest;
adding digesting buffer to the at least one excised sample;
determining the amount of the at least one protein of interest by analyzing the at least one digest mixture by mass spectrometry.

19. Use as claimed in claim 14, wherein a peptide mass fingerprinting is performed, in particular comprising the steps of
isolating a serum sample from a blood sample of a subject suffering from or being susceptible to cancer;
adding lysis buffer to the serum sample;
separating the proteins of the lysed serum sample by 2-D gel electrophoresis;
excising from the gel at least one sample containing a protein of interest;
adding digesting buffer to the at least one excised sample;
determining the amount of the at least one protein of interest by analyzing the at least one digest mixture by mass spectrometry.

20. Use of claim 1 comprising the steps of
isolating serum samples from blood samples of a plurality of c-myc cancer mice bearing the same type of tumor;
adding lysis buffer to said serum samples;
separating the proteins of said lysed serum samples by 2-DE gel electrophoreses;
excising from the gels each one sample containing a protein of interest,
adding digesting buffer to the excised samples;
analyzing the digest mixtures by mass spectrometry, in particular by peptide mass fingerprinting, and determining the protein of interest as biomarker, wherein a serum level of the protein being significantly higher and/or a serum level of the protein being significantly lower than the level of said protein in the serum of normal subjects is indicating the biomarker and/or
for identifying diagnostic dysplasia biomarkers, comprising the steps of
isolating serum samples from blood samples of a plurality of EGF cancer mice having the same type of abnormality in maturation of cells, in particular having low grade or high grade bronchial dysplasia;
adding lysis buffer to said serum samples;
separating the proteins of said lysed serum samples by 2-DE gel electrophoreses;
excising from the gels each one sample containing a protein of interest,
adding digesting buffer to the excised samples;
analyzing the digest mixtures by mass spectrometry, in particular by peptide mass fingerprinting, and determining the protein of interest as biomarker, wherein a serum level of the protein being significantly higher and/or a serum level of the protein being significantly lower than the level of said protein in the serum of normal subjects is indicating the biomarker.
